(19) United States
(12) Patent Application Publication
(10) Pub. No.: US 2019/0359992 A1
(43) Pub. Date: Nov. 28, 2019

(54) ALTERING EXPRESSION OF GENE PRODUCTS IN PLANTS THROUGH TARGETED INSERTION OF NUCLEIC ACID SEQUENCES

(71) Applicant: CELLECTIS, Paris (FR)

(72) Inventor: Nicholas Baltes, Maple Grove, MN (US)

(21) Appl. No.: 16/313,799

(22) PCT Filed: Jun. 28, 2017
(86) PCT No.: PCT/US2017/039641

§ 371 Date: Dec. 27, 2018

Related U.S. Application Data
(60) Provisional application No. 62/355,489, filed on Jun. 28, 2016.

(51) Int. Cl. C12N 15/82; C12N 15/52

(52) U.S. Cl. CPC C12N 15/8213; C12N 15/8275; C12Y 205/01019; C12N 15/52; C12N 15/8242

(57) ABSTRACT

Materials and methods for changing expression of a gene product in a plant are provided, and in an embodiment for creating herbicide tolerant plants are described herein. The methods provide for inserting into a plant genome, at a different locus than an endogenous gene, a genomic or coding sequence of the gene, which may be modified, into a genetic location that is different from the endogenous gene and where there is a desired transcriptional activity. The methods described herein can include the targeted insertion of an endogenous 5-enolpyruvylshikimate-3-phosphate synthase gene into a genomic locus that enables sufficient expression to confer herbicide tolerance.

### RELATED APPLICATION

This application claims priority to co-pending application U.S. Ser. No. 62/355,489 filed Jun. 28, 2016, the contents of which are incorporated herein by reference in its entirety

### SEQUENCE LISTING

The instant application contains a Sequence Listing which has been submitted in ASCII format via EFS-Web and is hereby incorporated by reference in its entirety. Said ASCII copy, created on Jun. 26, 2017, is named P12336WO00_Sequence_Listing_ST25.txt and is 170,550 bytes in size.

### TECHNICAL FIELD

This document relates to materials and methods for altering expression of gene product in plants, including creating herbicide tolerant plants. In an embodiment, this work relates to the targeted insertion of 5-enolpyruvylshikimate-3-phosphate synthase genes into genomic loci that enable sufficient expression to confer herbicide tolerance.

### BACKGROUND

Effective weed management is critical for achieving maximum crop growth and productivity. One management method is to spray herbicides that effectively target and kill weeds, but not crop plants. One of the most widely used herbicides for controlling weeds is N-(phosphonomethyl)glycine (commonly referred to as glyphosate). Glyphosate is a nonselective, broad-spectrum foliar herbicide that can control over 300 weed species. To introduce glyphosate tolerance, crop plant genomes can be modified with one or more gene(s) that encode enzymes that have reduced affinity for, or degrade, the herbicide.

Glyphosate functions as an herbicide by preventing phosphoenol pyruvate from binding to 5-enolpyruvylshikimate-3-phosphate synthase (EPSPS), thereby shutting down the shikimate pathway in plants. Glyphosate binds to EPSPS adjacent to shikimate 3-phosphate in a location that is normally the binding site for phosphoenol pyruvate. The binding of glyphosate to the phosphoenol pyruvate site mimics an intermediate state of the ternary enzyme-substrates complex (Schönbrunn et al., Proceedings of the National Academy of Sciences, 98: 1376-1380, 2001). By preventing the conversion of shikimate 3-phosphate to 5-enolpyruvylshikimate-3-phosphate, the plant is unable to produce necessary aromatic amino acids required for survival.

Significant effort has been invested in identifying glyphosate-insensitive EPSPS mutants that can be transferred to crops for providing resistance to herbicides. Several promising enzymes from bacteria and plants were identified through selective evolution, site-directed mutagenesis, and microbial screens (Comai et al., Science 221: 370-371, 1983; Padgette et al., J. Biol. Chem. 266: 22364-22369, 1991; Eschenburg et al., Arch. Biochem. Biophys. 282: 433-436, 2002). However, increased glyphosate tolerance was often accompanied by decreased affinity for phosphoenol pyruvate, thereby resulting in decreased EPSPS enzyme activity. One of the most widely used EPSPS mutants in plants is a bacterial EPSPS from Agrobacterium sp. strain CP4 (referred to herein as cp4 epsps). However, when introduced into plants, the resulting product contains a cross-kingdom bacterial gene—a trigger for regulation with many governmental agencies. In addition to CP4, a mutated EPSPS protein from Salmonella typhimurium strain CT7 confers glyphosate resistance to plant cells (U.S. Pat. Nos. 4,535,060; 4,769,061; and 5,094,945).

Efforts to modify endogenous plant EPSPS proteins have been met with limited success. Difficulty modifying plant EPSPS genes is exemplified by the limited number of reports describing naturally glyphosate-tolerant EPSPS enzymes in plants. It is believed that modifications to the glyphosate binding domain also have the negative effect of lowering the binding kinetics of phosphoenol pyruvate, subsequently lowering the catalytic activity of EPSPS (Kishore, G. and Shah, D. Ann. Rev. Biochem. 57:627-663, 1988). Approaches to introduce glyphosate tolerance by stepwise selection on the herbicide have resulted in plant suspension cell lines with significantly higher EPSPS activity levels, which is attributed to gene amplification (Widholm et al., Physiologia plantarum, 112:540-545. 2001 Shyr al., Molecular & general genetics, 232:377382, 1992).

Due to the agronomical advantages of herbicide resistant plants, additional methods for conferring herbicide resistance are desirable. Further, methods for generating herbicide tolerant crops without the use of bacterial genes or viral promoters would be beneficial for commercialization.

### SUMMARY

The present methods and products are based in part on the discovery that expression of plant genes can be altered by inserting a copy of the a nucleic acid sequence which comprises the genomic or coding sequence of plant genes into different genomic loci from the loci of the gene in the plant, wherein the copy of the genomic or coding sequence does not contain a promoter, and wherein the different genomic loci have transcriptional activity. An embodiment provides the sequence to be inserted can be endogenous, which can be obtained from a plant or synthetically created. A method for altering the expression of plant genes, including endogenous genes is provided, the method including providing a plant cell containing one or more endogenous plant genes, modifying the genetic material within said plant cell by inserting copy of a nucleic acid sequence comprising the genomic sequence or coding sequence of said plant gene, which can be in one embodiment a modified sequence, into a different genomic locus, wherein said different genomic locus comprises transcriptional activity, and growing the plant cells in which the integrated copy of said genomic sequence or coding sequence is transcribed or expressed. In some embodiments, the method can be accomplished by one of the following approaches: 5′ insertion, complete replacement, 3′ insertion, internal exon insertion, internal exon sequence replacement, internal intron insertion, and internal intron sequence replacement. In another embodiment, the method can further include a step of inactivating the endogenous plant gene at its original genomic locus. In another embodiment, the method can include a step of regenerating the modified plant cell into a plant part or plant.

The present method and products are also based in part on the discovery that herbicide tolerance can be introduced by inserting the genomic or coding sequence of one or more EPSPS plant genes into different genomic loci from the endogenous gene, where the different genomic loci have transcriptional activity. The processes features a method for making an herbicide tolerant plant, where the method includes providing a plant cell comprising one or more endogenous EPSPS genes, modifying the genetic material within said plant cell by inserting an endogenous EPSPS genomic sequence or coding sequence into a different genomic locus, wherein said different genomic locus comprises transcriptional activity, and growing the plant cells in which the inserted copy of said genomic sequence or coding sequence is transcribed or expressed. In some embodiments, the method can be accomplished by one of the following approaches: 5′ insertion, complete replacement, 3′ insertion, internal exon insertion, internal exon sequence replacement, internal intron insertion, and internal intron sequence replacement. In some embodiments, the method can include inserting a copy of the genomic sequence or coding sequence of an endogenous or modified EPSPS gene into a locus having a ubiquitin gene. In some embodiments, the method can include inserting a copy of the genomic sequence or coding sequence of an endogenous EPSPS gene into GmUbi3 as shown in SEQ ID NO:16, or sequence with at least 90% identity to SEQ ID NO:16. In some embodiments, the method can include inserting a copy of the genomic sequence or coding sequence of an endogenous or modified EPSPS gene into GmERF10 genomic sequence as shown in SEQ ID NO:17, or sequence with at least 90% identity to SEQ ID NO:17. In some embodiments, the method can include inserting more than one EPSPS coding sequences into different genomic loci. In some embodiments, the method can include inserting more than two EPSPS coding sequences into different genomic loci. In some embodiments, the method can include inserting more than three EPSPS coding sequences into different genomic loci. In some embodiments, the method can further include inactivating endogenous EPSPS plant genes at their original genomic loci. In some embodiments, the method can further include regenerating the modified plant cell into a plant part or plant.

In another embodiment, an herbicide tolerant plant, plant part, or plant cell which contains an insertion of EPSPS genomic sequence or coding sequence in a different genomic locus. In one embodiment the herbicide tolerant plant, plant part, or plant cell contains one or more additional insertions of EPSPS genomic sequence or coding sequence. In some embodiments, the herbicide tolerant plant, plant part, or plant cell can contain an inserted copy of the genomic sequence or coding sequence of an EPSPS gene in a locus having a ubiquitin gene. In some embodiments, the herbicide tolerant plant, plant part, or plant cell can include an inserted a copy of the genomic sequence or coding sequence of an EPSPS gene into GmUbi3 as shown in SEQ ID NO:16, or sequence with at least 90% identity to SEQ ID NO:16. In some embodiments, the herbicide tolerant plant, plant part, or plant cell can contain an inserted copy of the genomic sequence or coding sequence of an EPSPS gene into GmERF10 genomic sequence as shown in SEQ ID NO:17, or sequence with at least 90% identity to SEQ ID NO:17. In some embodiments, the herbicide tolerant plant, plant part, or plant cell can contain sequence as shown in SEQ ID NO:7, SEQ ID NO:8, SEQ ID NO:9, SEQ ID NO:10, SEQ ID NO:11, SEQ ID NO:12, SEQ ID NO:13, SEQ ID NO:14, and SEQ ID NO:15; or sequence with 90% identity to SEQ ID NO:7, SEQ ID NO:8, SEQ ID NO:9, SEQ ID NO:10, SEQ ID NO:11, SEQ ID NO:12, SEQ ID NO:13, SEQ ID NO:14, and SEQ ID NO:15.

In another aspect, this document features a seeds of an herbicide tolerant plant, plant part, or plant cell which have an insertion of EPSPS genomic sequence or coding sequence in a different genomic locus. The seeds can be non-transgenic.

In another aspect, this document features a method for generating an herbicide tolerant plant, where the method includes providing a plant cell containing one or more endogenous EPSPS genes, modifying the genetic material within said plant cell by inserting a mepsps genomic sequence or coding sequence into a different genomic locus, wherein the different genomic locus has transcriptional activity, and regenerating the modified plant cell into a plant part or plant. As described herein, “mepsps” refers to a modified version of EPSPS. mepsps can be genomic sequence or coding sequence (CDS). mepsps can harbor sequence alterations compared to the wild type EPSPS sequence. The sequence alterations can comprise any sequence change that results in one or more amino acid changes that affect the ability for glyphosate to bind to the EPSPS protein. In some embodiments, the method can include inserting more than one mepsps into different genomic loci. In some embodiments, the method can include inserting mepsps into the plant genome using one of the following approaches: 5′ insertion, complete replacement, 3′ insertion, internal exon insertion, internal exon sequence replacement, internal intron insertion, and internal intron sequence replacement. In some embodiments, the method can include inserting mepsps into a locus having a ubiquitin gene. In some embodiments, the method can include inserting a copy of the genomic sequence or coding sequence of an mepsps gene into GmUbi3 as shown in SEQ ID NO:16, or sequence with at least 90% identity to SEQ ID NO:16. In some embodiments, the method can include inserting a copy of the genomic sequence or coding sequence of an mepsps gene into GmERF10 genomic sequence as shown in SEQ ID NO:17, or sequence with at least 90% identity to SEQ ID NO:17.

In another aspect, there is provided an herbicide tolerant plant, plant part, or plant cell which contains an insertion of an mepsps genomic sequence or coding sequence in a different genomic locus. In one embodiment the herbicide tolerant plant, plant part, or plant cell contains one or more additional insertions of an mepsps genomic sequence or coding sequence. In some embodiments, the herbicide tolerant plant, plant part, or plant cell can contain an inserted copy of the genomic sequence or coding sequence of an mepsps gene in a locus having a ubiquitin gene. In some embodiments, the herbicide tolerant plant, plant part, or plant cell can include an inserted a copy of the genomic sequence or coding sequence of an mepsps gene into GmUbi3 as shown in SEQ ID NO:16, or sequence with at least 90% identity to SEQ ID NO:16. In some embodiments, the herbicide tolerant plant, plant part, or plant cell can contain an inserted copy of the genomic sequence or coding sequence of an mepsps gene into GmERF10 genomic sequence as shown in SEQ ID NO:17, or sequence with at least 90% identity to SEQ ID NO:17.

In another aspect, this document features seed of an herbicide tolerant plant, plant part, or plant cell which have an insertion of an mepsps genomic sequence or coding sequence in a different genomic locus. The seeds can be non-transgenic.

### DESCRIPTION OF DRAWINGS

FIG. 1 is graphic showing an illustration of the soybean EPSPS genomic sequence and coding sequence (CDS) from chromosome 1 (Glyma01g33660).

FIG. 2 is a graphic showing illustrations of seven different means to insert GmEPSPS coding sequence into a soybean gene with an expression profile of interest. The far left white rectangular box refers to position of the 5′ UTR, the far right rectangular white box refers to the position of the 3′ UTR, the black box marked EPSPS shows location of the inserted EPSPS sequence, the remaining boxes are coding regions and the lines between the boxes represent introns.

FIG. 3 is a graphic showing illustrations of seven different means to insert GmEPSPS genomic sequence into a soybean gene with an expression profile of interest. The far left white rectangular box refers to position of the 5′ UTR, the far right rectangular white box refers to the position of the 3′ UTR, the black boxes shows location of the inserted EPSPS genomic sequence, the remaining boxes are coding regions and the lines between the boxes represent introns

FIG. 4 is a graphic showing an illustration of the soybean ubiquitin 3 gene (GmUbi3) located on chromosome 20 (Glyma20g27950). The illustration depicts the binding sites of three TALEN pairs (GmUbi3_T1 through T3).

FIG. 5 is the sequence of the GmUbi3 gene. The coding sequence is indicated by black highlighting and white nucleotides. The 3′ UTR is indicated by grey highlighting. The TALEN binding sites are indicated with bold and underlined nucleotides. The extent of the homology present on the donor arms is indicated by forward slashes.

FIG. 6 is a graphic showing an illustration of the three donor molecules designed to knockin GmEPSPS, Bar, or YFP into the GmUbi3 gene (3′ insertion). Black lines in the 3′ UTR structure indicate the position of mismatches for preventing TALEN binding.

FIG. 7 is a graphic showing an illustration of the three genome edits after successful targeted knockin of EPSPS, Bar or YFP into GmUbi3. Also shown are the location and names of primers used to molecularly characterize the targeted knockin event.

FIG. 8 is an image of soybean cotyledons four days post bombardment of plasmid DNA containing GmUbi3 TALEN pairs and YFP donor molecules.

FIG. 9A-Bis a graphic showing an illustration of the genome edit after successful targeted knockin of YFP into GmUbi3 (A) and an image of PCR results using primers designed to detect the 5′ junction of the knockin event within soybean immature cotyledons (B). The same DNA was sampled nine times

FIG. 10A-B is a graphic showing an illustration of the genome edit after successful targeted knockin of YFP into GmUbi3 (A) and an image of PCR results using primers designed to detect the 3′ junction of the YFP knockin event within soybean immature cotyledons (B). The same DNA was sampled three times (i.e., three technical replicates).

FIG. 11 is a graphic showing an illustration of the genome edit after successful targeted knockin of YFP into GmUbi3. Also shown are Sanger sequencing results from the 5′ and 3′ junction PCRs shown in FIGS. 9 and 10 from the sample with TALEN pair T02.1 and the geminivirus YFP donor.

FIG. 12 is an image of YFP-positive callus cells 46 days post bombardment.

FIG. 13 is an image of soybean protoplast cells delivered TALEN pair T03.1 and the geminivirus YFP donor.

FIG. 14 is an image of PCR results using primers designed to detect the 5′ junction of the EPSPS, YFP and Bar knockin events within soybean protoplasts.

FIG. 15 is an image of YFP-positive soybean protoplasts transformed with TALEN pair T03.1 and the geminivirus YFP donor seven to eight days post transformation.

### DETAILED DESCRIPTION

The methods and products described herein relates to the finding that expression of plant genes can be altered by inserting the genomic or coding sequence of the plant genes into different genomic loci, where the different genomic loci have transcriptional activity. As a result of inserting the genomic or coding sequence of plant genes into different genomic loci, the expression profile of the plant genes can be altered such that they are similar to the expression profile of genes near the site of insertion.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

The processes and plants produced described herein in an embodiment are to a method of controlling expression of one or more products encoded by a nucleic acid molecule in a plant. Expression of the one or more products is changed from the expression compared to the wild-type plant. The wild-type plant referred to here is the plant which does not comprises the at least one nucleotide sequence of the gene that is inserted into at least one second location. The product of a nucleotide sequence typically will be an amino acid sequence encoded by the nucleotide sequence, but can also include where no polypeptide is produced, but rather, for example, RNA is encoded and which impacts other plant process or production of other polypeptides. The nucleic acid sequence encoding the product is referred to here as the nucleic acid sequence of interest. The nucleic acid sequence will comprise the genomic or coding sequences of the gene, and in preferred embodiments will not include the promoter of the gene. The methods in embodiments can include determining whether to increase or decrease expression of a product encoded by the nucleic acid molecule of interest and/or direct expression to a particular location of the plant or cause expression to occur at a particular time or condition, or when exposed to a composition of matter, that is to cause expression to be inducible, and selecting the change in transcription desired. A gene of the plant genome is then identified where the selected transcription activity occurs. The gene having the selected transcription activity in an embodiment is a gene that does not have the same promoter as the gene of the nucleic acid molecule of interest. A preferred location is identified for insertion of the nucleic acid molecule of interest so as to preserve the ability of the insert site to transcribe the inserted nucleic acid molecule of interest in the desire manner. The nucleic acid molecule of interest without a promoter is inserted at the target site.

Thus, an embodiment provides a method of changing expression of a gene product in a plant, which can include:
a) identifying a desired change in expression of a gene product in a plant and determining the transcription activity needed of a first gene in a first location encoding the gene product;
b) identifying at least one second location in the genome of said plant having said transcription activity;
c) inserting a nucleic acid sequence of said gene into said at least one second location, wherein said nucleic acid sequence does not comprise a promoter; and producing a plant that has changed expression of said gene product as a result of expression of said nucleic acid molecule.

In an embodiment, the nucleic acid molecule comprises a modified gene.

In certain embodiments, the changed expression is selected from increasing expression of said gene product, decreasing expression of said gene product, expressing said gene product at a higher level in select plant tissue than other plant tissue, expressing said gene product at a selected time or plant growth state, or expressing said gene product when induced by an inducer, or a combination thereof. Embodiments provide the second location has transcription activity that is selected from constitutive expression, plant tissue preferred expression, expressing the product at a lower or higher level than the wild-type gene; and expressing when exposed to an inducer, or a combination thereof.

In one embodiment, the methods provided herein include the insertion of one or more s plant herbicide-related nucleotide sequence into different genomic loci for the purpose of introducing herbicide tolerance, and, in in one embodiment, to increase glyphosate tolerance. When referring to an endogenous gene is meant the nucleic acid molecule comprises the sequence of the wild-type sequence occurring in the wild-type plant, or a sequence having a percent identity that allows it to retain the function of the encoded product, such as a sequence with at least 90% identity, and may be obtained from the plant or plant part of cell, or may be synthetically produced. Further embodiments provide the sequence has at least 91%, 92%, 93%, 94%, 95%, 96%, 97%, 98%, 99% identity. Embodiments provide the nucleotide sequence of interest is inserted at a different locus than that of the wild-type gene and which different locus does not comprise the promoter of the wild-type gene.

In an embodiment the herbicide tolerance is glyphosate tolerance. The biological target of glyphosate is an enzyme within the shikimate pathway, 5-enolpyruvylshikimate-3-phosphate synthase (EPSPS). Binding of glyphosate to EPSPS results in inactivation of the protein, and subsequent inactivation of the shikimate pathway. In plants, bacteria, fungi and algae, the shikimate pathway is responsible for the biosynthesis of chorismate, which is the precursor for the aromatic amino acids phenylalanine, tyrosine and tryptophan. This pathway is absent from animals, and products of the shikimate pathway must be obtained in the animal's diet. The first reaction in this pathway is the transfer of a phosphate group from ATP to shikimate by shikimate kinase to produce shikimate 3-phosphate. The shikimate 3-phosphate is then transformed into 5-enolpyruvylshikimate-3-phosphate by the EPSPS enzyme. Transformation proceeds by EPSPS binding to shikimate 3-phosphate and phosphoenol pyruvate, and the subsequent transfer of the enolpyruvyl moiety of PEP to the 5-hydroxyl group of shikimate 3-phosphate. Finally, 5-enolpyruvylshikimate-3-phosphate is converted to chorismate by chorismate synthase.

EPSPS protein is found within plant cells and is encoded by one or more genes. For example, Glycine max contains two EPSPS genes (herein referred to as GmEPSPS), one on chromosome 1 (GmEPSPS chr 1) and the other on chromosome 3 (GmEPSPS chr 3). The genomic sequences for GmEPSPS chr 1 and GmEPSPS chr 3, from start codon to stop codon, are provided within SEQ ID NO:1 and SEQ ID NO:3, respectively. The coding sequence for GmEPSPS chr 1 and GmEPSPS chr 3, from start codon to stop codon, is provided within SEQ ID NO:2 and SEQ ID NO:4, respectively. EPSPS protein can be found in other economically valuable crop plants, including wheat (Triticum aestivum), rice (Oryza sativa), canola (Brassica napus), potato (Solanum tuberosum) and alfalfa (Medicago sativa). For example, the Triticum aestivum (AABBDD) EPSPS genes are present on chromosomes 7A (TaEPSPS-7A1; Accession KP411547.1), 7D (TaEPSPS-7AD; Accession KP411548.1) and 4A (TaEPSPS-4A1; Accession KP411549.1); for the molecular characterization of the EPSPS genes, see Aramrak et al., BMC Genomics, 16:844, 2015. Further, the Oryza sativa EPSPS gene is located on chromosome 6 (Accession AF413081.1); for the molecular characterization of the EPSPS gene, see Xu et al, Acta Botanica Sinica, 44:188-192, 2002. An alignment of sample EPSPS proteins of Arabidopsis, maize, wheat, rice and soybean shows 81.1% identity.

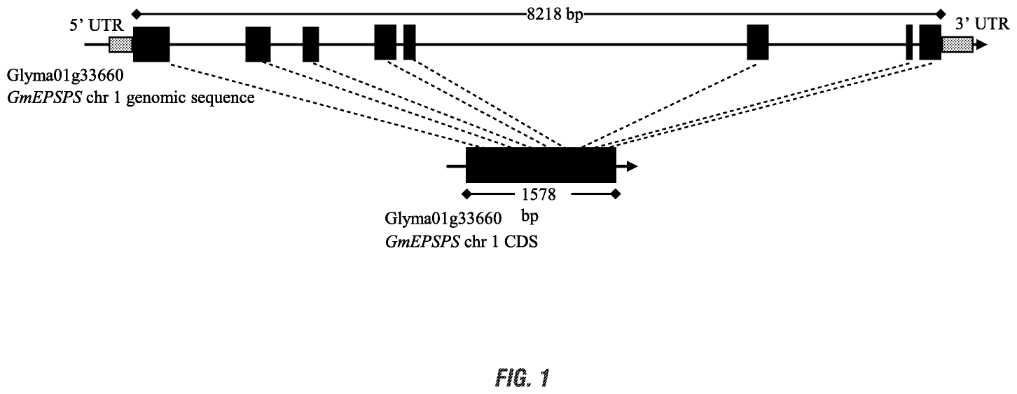

In one embodiment, the GmEPSPS chr 1 genomic sequence or coding sequence (CDS) can be used to confer herbicide tolerance (FIG. 1). The GmEPSPS chr 1 genomic sequence has eight exons and seven introns, and has a total length of 8,218 bp from start codon to stop codon (SEQ ID NO:1). The GmEPSPS chr 1 CDS does not contain native introns, and has a total length of 1,578 bp (SEQ ID NO:2). Both the genomic sequence and CDS for GmEPSPS chr 1 code for the same protein (SEQ ID NO:5).

In another embodiment, the GmEPSPS chr 3 genomic sequence or coding sequence (CDS) can be used to confer herbicide tolerance. The GmEPSPS chr 3 genomic sequence has eight exons and seven introns, and has a total length of 7,534 bp from start codon to stop codon (SEQ ID NO:3). The GmEPSPS chr 3 CDS does not contain native introns, and has a total length of 1,581 bp (SEQ ID NO:4). Both the genomic sequence and CDS for GmEPSPS chr 3 code for the same protein (SEQ ID NO:6). The percent identity of these soybean EPSPS protein is 96.2%.

The present invention relates to the discovery that endogenous plant promoters can be used to confer desire gene product expression, and, in an embodiment, herbicide tolerance. In its native configuration, the EPSPS gene in crop plants has insufficient transcriptional activity to confer field level tolerance to glyphosate. We discovered that transfer of the EPSPS genomic sequence or CDS to a different location within the host's genome can enable sufficient expression of EPSPS for conferring herbicide tolerance. More specifically, the GmEPSPS genomic sequence or CDS can be inserted downstream of different Glycine max genes or promoters. The GmEPSPS genomic sequence or CDS can be the wild type GmEPSPS genomic sequence or CDS. Alternatively, the GmEPSPS genomic sequences or CDS can be a modified GmEPSPS genomic sequences or CDS (referred to as Gmmepsps), such that Gmmepsps has reduced affinity for glyphosate.

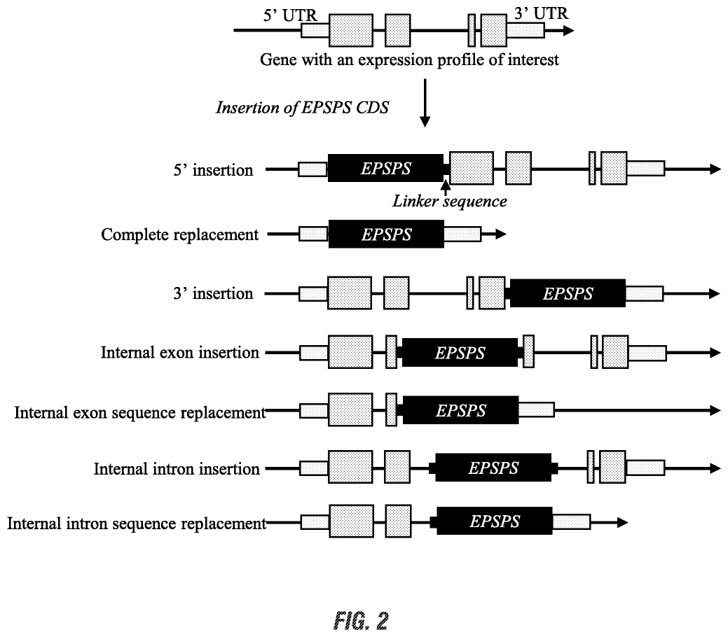

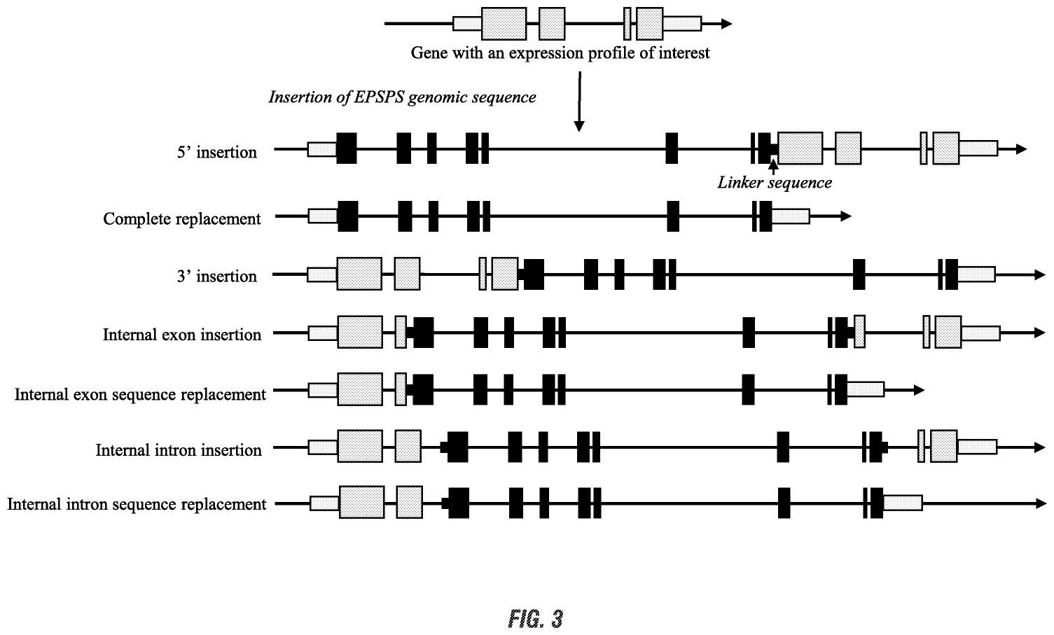

The present processes and plants also relate to identifying regions within plant genomes for inserting genomic sequences or coding sequences of interest. To this end, the first step is to understand the desired expression characteristics of the genomic sequence of interest. For example, concerning GmEPSPS, a strong promoter with ubiquitous expression may be desired. By way of further example, without limitation, where the gene is a resistance (R) gene, it may be desired to provided weaker expression compared to that achieved with the native R gene promoter. R genes play a role in plant immunity with the potential cost of reduced fitness (Tian et al., Nature 423:74-77, 2003). Optimization of R-gene expression can subsequently result in optimized fitness and defense response. In other situations tissue specific, stage specific or inducible expression may be desired. The second step is to identify an endogenous gene that matches the desired expression profile. Several methods and software programs are available for identifying genes with desired expression characteristics. These include, but are not limited to RNA-sequencing (whole transcriptome shotgun sequencing); see, for example, Wang et al., Nature Reviews Genetics, 10:57-63, 2009. Once genes with desired expression profiles are identified, it is to be understood that the promoter sequence (usually upstream or nearby the gene of interest) is a key component used in the method, as opposed to the actual gene being expressed by the promoter. The last step is to determine the specific type of genome edit that is required to capture the transcriptional activity of the identified promoter (FIGS. 2 and 3). This last step is explained in more detail within the following teachings.

The present processes and plants relate to methods for inserting sequence into the plant genome and capturing nearby promoter activity. Several different means were identified for inserting genomic sequences or CDSs of interest into a region within the plant genome for the purpose of capturing the transcriptional activity of a nearby promoter (FIGS. 2 and 3). The first means is 5′ insertion. 5′ insertion involves the insertion of the genomic sequence or CDS of interest (including, but not limited to, GmEPSPS genomic sequence or CDS) into the 5′ region of a gene sequence near a promoter of interest, where the gene sequence encodes a functional RNA or protein. There optionally can be a linker sequence between the GmEPSPS genomic sequence or CDS and the gene sequence. The linker sequence can be, for example, a 2A sequence, IRES, or transcriptional termination sequence. The linker sequence can also be intron acceptor sequence or intron donor sequence. The linker sequence can also be no sequence. A second means is complete replacement. Complete replacement involves the complete replacement of a gene sequence near a promoter of interest by the GmEPSPS genomic sequence or CDS. A third means is 3′ insertion. 3′ insertion involves the insertion of the GmEPSPS genomic sequence or CDS into the 3′ region of a gene sequence near a promoter of interest. A promoter of interest is the promoter identified to provide the type of expression of the inserted sequence as discussed herein. If a stop codon is present within the gene sequence, then the insertion must occur before the stop codon. There optionally can be a linker sequence between the gene sequence and the GmEPSPS genomic sequence or CDS. The fourth means is internal exon insertion. Internal exon insertion involves the insertion of the GmEPSPS genomic sequence or CDS into an exon within a gene sequence near a promoter of interest. There optionally can be linker sequences at the sites of insertion at both the 5′ and 3′ end of the gene sequence. The fifth means is internal exon sequence replacement. Internal exon sequence replacement involves the insertion of the GmEPSPS genomic sequence or CDS of interest into an exon within a gene sequence and also the removal of downstream gene sequence. The sixth means is internal intron insertion. Internal intron insertion involves the insertion of the GmEPSPS genomic sequence or CDS into an intron within a gene sequence near a promoter of interest that encodes a functional RNA or protein. There optionally can be linker sequences at the site of insertion at both the 5′ and 3′ end of the GmEPSPS genomic sequence or CDS. Preferably, the linker sequences consist of splice acceptor and/or donor sequences. The seventh means is internal intron sequence replacement. Internal intron sequence replacement involves the insertion of the GmEPSPS genomic sequence or CDS into an intron within a gene sequence and also the removal of downstream gene sequence. Notably, if the gene sequence near the promoter of interest encodes a functional RNA or protein that is essential for plant growth or affects plant physiology, then it is beneficial to perform gene edits such that they will not destroy gene function. In that instance, it may be preferable to perform either 5′ insertion or 3′ insertion.

In one embodiment, the methods provided herein can involve the targeted insertion of multiple endogenous EPSPS genomic sequences or CDSs. In one instance, and most preferably, a single EPSPS genomic sequence or CDS is inserted into a genomic locus with sufficient expression to confer glyphosate tolerance. In another instance, two EPSPS genomic sequences or CDSs are inserted into two different genomic loci with sufficient expression to confer glyphosate tolerance. In another instance, three EPSPS genomic sequences or CDSs are inserted into three different genomic loci with sufficient expression to confer glyphosate tolerance. In another instance, four EPSPS genomic sequences or CDSs are inserted into four different genomic loci with sufficient expression to confer glyphosate tolerance. In another instance, five EPSPS genomic sequences or CDSs are inserted into five different genomic loci with sufficient expression to confer glyphosate tolerance. In another instance, six EPSPS genomic sequences or CDSs are inserted into six different genomic loci with sufficient expression to confer glyphosate tolerance. In another instance, seven EPSPS genomic sequences or CDSs are inserted into seven different genomic loci with sufficient expression to confer glyphosate tolerance. In another instance, eight EPSPS genomic sequences or CDSs are inserted into eight different genomic loci with sufficient expression to confer glyphosate tolerance. In another instance, nine EPSPS genomic sequences or CDSs are inserted into nine different genomic loci with sufficient expression to confer glyphosate tolerance. In another instance, ten EPSPS genomic sequences or CDSs are inserted into ten different genomic loci with sufficient expression to confer glyphosate tolerance. In another instance, the insertions described in this paragraph can comprise a mepsps genomic sequence or coding sequence. In another embodiment, more than ten EPSPS genomic sequences or CDSs are inserted into more than ten different genomic loci with sufficient expression to confer glyphosate tolerance. A still further embodiment provides for one, two, three, four, five, six, seven, eight, nine, ten or more of the EPSPS genomic sequences inserted in a different genetic loci from the endogenous gene, where all of the inserted EPSPS genomic sequences are inserted into the same different genetic loci.

In one embodiment, the methods provided herein can involve the targeted knockout of the original endogenous EPSPS gene or genes after the targeted insertion of EPSPS genomic or CDS into a locus with a gene sequence near a promoter of interest. Knocking out the original endogenous EPSPS gene or genes can result in all EPSPS gene expression being controlled from the promoter of interest. In addition, gene knockdown using RNAi technology can be employed. Teachings for performing gene knockout and gene knockdown in plants can be found, for example, in Haun et al., Plant Biotechnology Journal, 12:934-940, 2014 and Gil-Humanes et al., Proceedings of the National Academy of Sciences, 107:17023-17028, 2010.

In one embodiment, the methods provided herein can be used to generate herbicide tolerant plants by insertion of a plant EPSPS into a gene with a promoter of interest. The promoter of interest is that endogenous promoter having the described transcriptional activity within the plant genome. The EPSPS sequence to be inserted can be genomic sequence which includes the introns and exons, or the EPSPS sequence can be CDS. The EPSPS genomic sequence or CDS can be a wild type plant EPSPS genomic sequence or CDS, or the EPSPS genomic sequence or CDS can be a modified plant EPSPS genomic sequence or CDS (mepsps) and can be obtained in any convenient manner, whether isolated from the plant or synthetically produced, for example.

As described herein, “mepsps” refers to a modified version of EPSPS. mepsps can be genomic sequence or CDS. mepsps can harbor sequence alterations compared to the wild type EPSPS sequence. The sequence alterations can comprise any sequence change that results in one or more amino acid changes that affect the ability for glyphosate to bind to the EPSPS protein. A preferred embodiment provides the mepsps retains the ability when expressed in a plant to provide tolerance to glyphosate, and in a further embodiment provides increased tolerance and/or improved plant function or health compared to a plant expressing an unmodified EPSPS sequence.

Modifications that reduce affinity to glyphosate but retains EPSPS function can also be used. See in particular FIG. 1 of U.S. Pat. No. 5,866,775, where alignment of the amino acid sequence for EPSPS synthase from various plant and bacterial species is shown. Examples of other modified EPSPS include those shown is U.S. Pat. No. 5,310,667 (changing alanine for glycine between positions 120 and 160 and aspartic acid or asparagine for glycine between positions 120 and 160 in the mature wild type EPSPS) U.S. Pat. No. 6,225,114 (changing an alanine for glycine at the conserved sequence between positions 80 and 120 and threonine for alanine between positions 170 and 210), U.S. Pat. No. 5,866,775 (changing alanine for glycine between positions 80 and 120 and threonine for alanine between positions 170 and 210), U.S. Pat. Nos. 6,566,587 and 6,040,497 (changing threonine to isoleucine at position 102 and proline to serine at position 106). See also U.S. Pat. No. 7,045,684 including discussion of substitutions at residues 177 (changing isoleucine for threonine and 182 (changing serine for proline) in Arabidopsis and 179 (changing isoleucine for threonine) and 183 (changing serine for proline in Arabidopsis). Each of these references are incorporated herein by reference in its entirety. Amino acid numbering is relative to the start of the mature EPSPS protein in plants. The mature EPSPS protein is produced after removal of the chloroplastic transit signal peptide (cTP), located at the N-terminus of the full length EPSPS protein (Della-Cioppa et al., Proc Natl Acad Sci USA 83:6873-6877, 1986). In one embodiment, the mepsps comprises a sequence, when aligned with SEQ ID NO: 5 or 6, comprises between residues 80 and 200, at least one modified residue, two modified residues, or more and provides increased glyphosate tolerance and/or plant health. See SEQ ID NO: 30 and 31 for a modified mepsps of SEQ ID NO: 5 and 6 respectively with the modification of an isoleucine at residue 102 and serine at residue 106.

In one embodiment, methods provided herein include inserting into the genome of a plant an mepsps DNA coding sequence or genomic sequence encoding a glyphosate tolerant EPSPS protein having an isoleucine or leucine at position 102, and an amino acid at position 106 selected from the group consisting of threonine, glycine, cysteine, alanine, and isoleucine. In another embodiment, the product provided includes a plant than contains the mepsps DNA coding sequence or genomic sequence is tolerant to glyphosate herbicide. As referred to herein, “TIPS” or “mepsps TIPS” refers to a modified version of the EPSPS protein that contains a threonine to isoleucine and a proline to serine mutation. By way of example, below is the location within the soybean EPSPS polypeptide of the modified residues at positions 102 and 106. Both SEQ ID NO: 5 and SEQ ID NO: 6 have the same amino acid in this region.

| (SEQ ID NO: 29) | |
|---|---|
| | T102 P106 |
| | ↓ ↓ |
| GmEpsps | GNAGTAMRPLTAAVVAAGG |

In one embodiment, the methods provided herein include the physical insertion of EPSPS genomic or CDS into a target locus with a promoter of interest. There are two different means to physically insert EPSPS sequence within a target locus. The first means includes homologous recombination of genomic sequence at or near the target locus with a donor molecule containing EPSPS sequence. This first means can also include the delivery of a sequence-specific nuclease that targets and cleaves genomic DNA at or near the target locus. The donor molecule can include EPSPS sequence that is flanked by arms that are homologous to sequence at or near the target locus. The donor molecule can include EPSPS sequence that is adjacent to one arm that is homologous to sequence at or near the target locus with the promoter of interest. The arms of homology can include sequence >90% similar to the target locus. The individual arms of homology can be between 10 and 10,000 base pairs or more. The donor molecule can be single-stranded DNA. The donor molecule can be double-stranded DNA. The donor molecule can be circular DNA. The donor molecule can be linear DNA. The second means involves the use of the non-homologous end joining pathway to directly insert EPSPS sequence into the plant genome. Instead of providing a donor molecule containing flanking arms of homology, a double-stranded DNA molecule encoding EPSPS is provided. The DNA molecule encoding EPSPS can be linear DNA. The DNA molecule encoding EPSPS can be circular DNA. The DNA molecule can include EPSPS or mepsps genomic sequence or coding sequence flanked by sequence-specific nuclease target sites. The second means includes delivery of a double-stranded DNA molecule and one or more sequence-specific nucleases that bind and cleave genomic DNA at or near the target locus.

The term “rare-cutting endonuclease” or “sequence-specific endonuclease” as used herein refers to a natural or engineered protein having endonuclease activity directed to a nucleic acid sequence with a recognition sequence (target sequence) about 12-40 bp in length (e.g., 14-40, 15-36, or 16-32 bp in length; see, e.g., Baker, Nature Methods 9:23-26, 2012). Typical rare-cutting endonucleases cause cleavage inside their recognition site, leaving 4 nt staggered cuts with 3′-OH or 5′-OH overhangs. In some embodiments, a rare-cutting endonuclease can be a meganuclease, such as a wild type or variant homing endonuclease (e.g., a homing endonuclease belonging to the dodecapeptide family (LAGLIDADG; SEQ ID NO:9) (see, WO 2004/067736). In some embodiments, a rare-cutting endonuclease can be a fusion protein that contains a DNA binding domain and a catalytic domain with cleavage activity. TALE-nucleases and zinc finger nucleases (ZFNs) are examples of fusions of DNA binding domains with the catalytic domain of an endonuclease such as FokI. Customized TALE-nucleases are commercially available under the trade name TALEN™ (Cellectis, Paris, France).

TALEs are found in plant pathogenic bacteria in the genus Xanthomonas. These proteins play important roles in disease, or trigger defense, by binding host DNA and activating effector-specific host genes (see, e.g., Gu et al., Nature 435:1122-1125, 2005; Yang et al., Proc. Natl. Acad. Sci. USA 103:10503-10508, 2006; Kay et al. Science 318:648-651, 2007; Sugio et al., Proc. Natl. Acad. Sci. USA 104:10720-10725, 2007; and Römer et al. Science 318:645-648, 2007). Specificity depends on an effector-variable number of imperfect, typically 34 amino acid repeats (Schornack et al., J. Plant Physiol. 163:256-272, 2006; and WO 2011/072246). Polymorphisms are present primarily at repeat positions 12 and 13, which are referred to herein as the repeat variable-diresidue (RVD).

The RVDs of TALEs correspond to the nucleotides in their target sites in a direct, linear fashion, one RVD to one nucleotide, with some degeneracy and no apparent context dependence. This mechanism for protein-DNA recognition enables target site prediction for new target specific TALEs, as well as target site selection and engineering of new TALEs with binding specificity for the selected sites.

TALE DNA binding domains can be fused to other sequences, such as endonuclease sequences, resulting in chimeric endonucleases targeted to specific, selected DNA sequences, and leading to subsequent cutting of the DNA at or near the targeted sequences. Such cuts (double-stranded breaks) in DNA can induce mutations into the wild-type DNA sequence via NHEJ or homologous recombination, for example. In some cases, TALE-nucleases can be used to facilitate site directed mutagenesis in complex genomes, knocking out or otherwise altering gene function with great precision and high efficiency. As described in the Examples below, TALE-nucleases targeted to the Nicotiana benthamiana ALS gene can be used to mutagenize the endogenous gene, confirmed by indels at the target site. The fact that some endonucleases (e.g., FokI) function as dimers can be used to enhance the target specificity of the TALE-nuclease. When the two TALE-nuclease recognition sites are in close proximity the inactive monomers can come together to create a functional enzyme that cleaves the DNA. By requiring DNA binding to activate the nuclease, a highly site-specific restriction enzyme can be created.

By way of example, a method using TALENs for modifying the genetic material of a cell, can include (a) providing a cell containing a target DNA sequence; and (b) introducing a transcription activator-like (TAL) effector-DNA modifying enzyme into the cell, the TAL effector-DNA modifying enzyme comprising (i) a DNA modifying enzyme domain that can modify double stranded DNA, and (ii) a TAL effector domain comprising a plurality of TAL effector repeat sequences that, in combination, bind to a specific nucleotide sequence in the target DNA sequence, such that the TAL effector-DNA modifying enzyme modifies the target DNA within or adjacent to the specific nucleotide sequence in the cell or progeny thereof. The method can further include providing to the cell a nucleic acid comprising a sequence homologous to at least a portion of the target DNA sequence, such that homologous recombination occurs between the target DNA sequence and the nucleic acid. The target DNA can be chromosomal DNA. The introducing can comprise transfecting the cell with a vector encoding the TAL effector-DNA modifying enzyme, mechanically injecting the TAL effector-DNA modifying enzyme into the cell as a protein, delivering the TAL effector-DNA modifying enzyme into the cell as a protein by means of the bacterial type III secretion system, or introducing the TAL effector-DNA modifying enzyme into the cell as a protein by electroporation. The DNA modifying enzyme can be an endonuclease (e.g., a type II restriction endonuclease, such as FokI).

The TAL effector domain that binds to a specific nucleotide sequence within the target DNA can comprise 10 or more DNA binding repeats, and preferably 15 or more DNA binding repeats. Each DNA binding repeat can include a repeat variable-diresidue (RVD) that determines recognition of a base pair in the target DNA sequence, wherein each DNA binding repeat is responsible for recognizing one base pair in the target DNA sequence, and wherein the RVD comprises one or more of: HD for recognizing C; NG for recognizing T; NI for recognizing A; NN for recognizing G or A; NS for recognizing A or C or G or T; N* for recognizing C or T, where * represents a gap in the second position of the RVD; HG for recognizing T; H* for recognizing T, where * represents a gap in the second position of the RVD; IG for recognizing T; NK for recognizing G; HA for recognizing C; ND for recognizing C; HI for recognizing C; HN for recognizing G; NA for recognizing G; SN for recognizing G or A; and YG for recognizing T. Each DNA binding repeat can comprise a RVD that determines recognition of a base pair in the target DNA sequence, wherein each DNA binding repeat is responsible for recognizing one base pair in the target DNA sequence, and wherein the RVD comprises one or more of: HA for recognizing C; ND for recognizing C; HI for recognizing C; HN for recognizing G; NA for recognizing G; SN for recognizing G or A; YG for recognizing T; and NK for recognizing G, and one or more of: HD for recognizing C; NG for recognizing T; NI for recognizing A; NN for recognizing G or A; NS for recognizing A or C or G or T; N* for recognizing C or T, wherein * represents a gap in the second position of the RVD; HG for recognizing T; H* for recognizing T, wherein * represents a gap in the second position of the RVD; and IG for recognizing T.

Further embodiments of using TALENs include providing a method for generating a nucleic acid encoding a TAL effector specific for a selected nucleotide sequence, comprising: (1) linearizing a starter plasmid with PspXI, the starter plasmid comprising a nucleotide sequence that encodes a first TAL effector DNA binding repeat domain having a repeat variable-diresidue (RVD) specific for the first nucleotide of the selected nucleotide sequence, wherein the first TAL effector DNA binding repeat domain has a unique PspXI site at its 3′ end; (2) ligating into the starter plasmid PspXI site a DNA module encoding one or more TAL effector DNA binding repeat domains that have RVDs specific for the next nucleotide(s) of the selected nucleotide sequence, wherein the DNA module has XhoI sticky ends; and (3) repeating steps (1) and (2) until the nucleic acid encodes a TAL effector capable of binding to the selected nucleotide sequence. The method can further comprise, after the ligating, determining the orientation of the DNA module in the PspXI site. The method can comprise repeating steps (1) and (2) from one to 30 times.

Still further TALEN methods are to generating a nucleic acid encoding a transcription activator-like effector endonuclease (TALEN), comprising (a) identifying a first nucleotide sequence in the genome of a cell; and (b) synthesizing a nucleic acid encoding a TALEN that comprises (i) a plurality of DNA binding repeats that, in combination, bind to the first unique nucleotide sequence, and (ii) an endonuclease that generates a double-stranded cut at a position within or adjacent to the first nucleotide sequence, wherein each DNA binding repeat comprises a RVD that determines recognition of a base pair in the target DNA, wherein each DNA binding repeat is responsible for recognizing one base pair in the target DNA, and wherein the TALEN comprises one or more of the above or other identified RVDs.

In an example of further methods available, the first nucleotide sequence can meet at least one of the following criteria: i) is a minimum of 15 bases long and is oriented from 5′ to 3′ with a T immediately preceding the site at the 5′ end; ii) does not have a T in the first (5′) position or an A in the second position; iii) ends in T at the last (3′) position and does not have a G at the next to last position; and iv) has a base composition of 0-63% A, 11-63% C, 0-25% G, and 2-42% T. The method can comprise identifying a first nucleotide sequence and a second nucleotide sequence in the genome of the cell, wherein the first and second nucleotide sequences meet at least one of the criteria set forth above and are separated by 15-18 bp. The endonuclease can generate a double-stranded cut between the first and second nucleotide sequences. Examples of methods of using TALENs may be found at Voytas et al., U.S. Pat. No. 8,697,853, incorporated herein by reference in its entirety.

In some embodiments, the methods provided herein can include the use of programmable RNA-guided endonucleases, or portions (e.g., subunits) thereof. RNA-guided endonucleases are a genome engineering tool that has been developed based on the RNA-guided CRISPR (Clustered Regularly Interspaced Short Palindromic Repeats)-associated nuclease (Cas9) from the type II prokaryotic CRISPR adaptive immune system (see, e.g., Belahj et al., Plant Methods 9:39, 2013). This system can cleave DNA sequences that are flanked by a short sequence motif known as a proto-spacer adjacent motif (PAM). Cleavage is achieved by engineering a specific CRISPR RNA (crRNA) that contains sequence that is complementary to the target sequence. The crRNA then base pairs with a trans-activating crRNA (tracrRNA) to form a cr/tracrRNA complex, which acts as a guide RNA that directs the Cas9 endonuclease to the cognate target sequence. A synthetic single guide RNA (sgRNA), which is a fusion between the crRNA and tracrRNA, can be employed, that, on its own, is capable of targeting the Cas9 endonuclease. Examples, without intending to be limiting, of CRISPR-Cas systems include U.S. Pat. No. 8,698,359 and US Published Applications 2015/0247150 and 2014/0068797, the contents of which are incorporated herein by reference in their entirety.

Another programmable RNA-guided endonuclease of a class 2 CRISPR-Cas system also has been described and used for gene editing purposes (Zetsche et al., Cell 163:759-771, 2015). This system uses a non-specific endonuclease unit from the Cpf1 protein family, with a specificity of cleavage conferred by a single crRNA (lacking tracr RNA). Similar to Cas9, the Cpf1 coding sequence can be fused to UTR sequences described herein to improve its stability, and thus the efficiency of the resulting gene editing method.

In one embodiment, the methods described herein involve the delivery of genome engineering reagents to plant cells, and regeneration of modified plants. Any suitable method can be used to introduce the nucleic acid into the plant cell. In some embodiments, for example, a method as provided herein can include contacting a plant cell with an organism that is capable of horizontal gene transfer (e.g., a bacterium, such as an Agrobacterium), where the organism contains a Ti or Ri plasmid, or T-DNA plasmid having a T-DNA region that includes the promoter, UTRs, coding sequence, and a poly-A tail. Methods for Agrobacterium-mediated transformation in wheat are described in Sparks et al., Methods in Molecular Biology, 1099:235-250, 2014. Methods for Agrobacterium-mediated transformation in soybean are described in Yamada et al., Breeding Science, 61:480-494, 2012. Methods for Agrobacterium-mediated transformation in potato are described in Beaujean et al., Journal of Experimental Botany, 49:1589-1595, 1998. In other embodiments, a method for introducing genome editing reagents as provided herein can include biolistic transformation. Methods for biolistic transformation for wheat are described in Sparks et al., Methods in Molecular Biology, 478:71-92, 2009. Methods for biolistic transformation for soybean are described in Rech et al., Nature Protocols, 3:410-418, 2008. Methods for biolistic transformation for potato are described in Ercolano et al., Molecular Breeding, 13:15-22, 2004. In other embodiments, methods for introducing genome editing reagents can include electroporation-mediated transformation of plant cells (e.g., protoplasts) or polyethylene glycol-mediated transformation of plant cells. Methods for isolation, culture and regeneration of potato plants from potato protoplasts is described in Jones et al., Plant Cell Reports, 8:307-311, 1989. Methods for isolation, transformation and regeneration of rice plants from rice protoplasts is described in Hayashimoto et al., Plant Physiology, 93:857-863, 1990.

The term introduced in the context of inserting a nucleic acid into a cell, includes transfection or transformation or transduction and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA). When referring to introduction of a nucleotide sequence into a plant is meant to include transformation into the cell, as well as crossing a plant having the sequence with another plant, so that the second plant contains the heterologous sequence, as in conventional plant breeding techniques. Such breeding techniques are well known to one skilled in the art. For a discussion of plant breeding techniques, see Poehlman (1995) Breeding Field Crops. AVI Publication Co., Westport Conn, 4th Edit. Backcrossing methods may be used to introduce a gene into the plants. This technique has been used for decades to introduce traits into a plant. An example of a description of this and other plant breeding methodologies that are well known can be found in references such as Poehlman, supra, and Plant Breeding Methodology, edit. Neal Jensen, John Wiley & Sons, Inc. (1988). In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a plant is obtained wherein essentially all of the desired morphological and physiological characteristics of the recurrent parent are recovered in the converted plant, in addition to the single transferred gene from the nonrecurrent parent. In one embodiment, the methods described herein involve the identification of the intended gene edit. It is to be understood that any method of identifying the site of insertion desired may be used and the following is provided by way of example. Several means can be employed to identify the desired targeted insertion. One means is by polymerase chain reaction (PCR). Here, primers are designed to detect the targeted insertion by amplifying the 5′ junction or 3′ junction. The 5′ junction and 3′ junction refers to a segment of genomic DNA, following true homologous recombination, which includes the junction of the genomic DNA with the homology carried by donor DNA. The PCR product can be cloned and sequenced using standard DNA sequencing techniques to verify successful targeted insertion. Another means to identify successful gene edits is by Southern blotting. For Southern blotting protocols, see, for example, Southern, Nature Protocols, 1:518-525, 2006.

Plants are substantially “tolerant” to a relevant herbicide when the plants requires more herbicide than non-tolerant like plants in order to produce a given herbicidal effect, or where the adverse impact of the herbicide is reduced compared to a plant than non-tolerant plants, or where the plant is resistant to a relevant herbicide. Plants that are substantially “resistant” to the herbicide exhibit few, if any, necrotic, lytic, chlorotic or other lesions, when subjected to herbicide at concentrations and rates which are typically employed by the agrochemical community to kill weeds in the field. As referred to herein, herbicide tolerant and herbicide resistant refers to the ability of a plant to tolerate the presence of an herbicide more effectively than a plant that is not herbicide tolerant or herbicide resistant. Plants which are resistant to an herbicide are also tolerant of the herbicide. Further, the term “tolerance” as used herein refers to a plant that is tolerant or resistant to the herbicide glyphosate. Tolerance can be determined by subjecting modified mutant and wild-type plants to a range of glyphosate in lethal and sub-lethal doses. The dose required to reduce shoot weight by 50% is then used to determine the resistant to susceptible (R/S) ratio.

The processes described here are useful in changing expression of any plant regarding tolerance to an herbicide. A person skilled in the art appreciates there are a wide variety of genes that can be employed for herbicide tolerant plant production. Glyphosate tolerance genes as discussed herein, provide tolerance imparted by mutant 5-enolpyruvl-3-phosphikimate synthase (EPSPS). See, for example, U.S. Pat. No. 4,940,835 to Shah et al., which discloses the nucleotide sequence of a form of EPSPS which can confer glyphosate tolerance. U.S. Pat. No. 5,627,061 to Barry et al. also describes genes encoding EPSPS enzymes. See also as examples, U.S. Pat. Nos. 6,248,876; 6,040,497; 5,804,425; 5,633,435; 5,145,783; 4,971,908; 5,312,910; 5,188,642; 4,940,835; 5,866,775; 6,225,114 B1; 6,130,366; 5,310,667; 4,535,060; 4,769,061; 5,633,448; 5,510,471; and 5,491,288. Glyphosate tolerance is also imparted to plants that express a gene that encodes a glyphosate oxido-reductase enzyme as described more fully in U.S. Pat. Nos. 5,776,760 and 5,463,175. In addition, glyphosate tolerance can be imparted to plants by the over expression of genes encoding glyphosate N-acetyltransferase. See, for example, U.S. Pat. No. 7,462,481. Also, aroA genes and other phosphono compounds such as glufosinate (phosphinothricin acetyl transferase (PAT) and Streptomyces hygroscopicus phosphinothricin acetyl transferase (bar) genes), and pyridinoxy or phenoxy proprionic acids and cyclohexones (ACCase inhibitor-encoding genes) can be used to produce herbicide tolerant plants. Another example involves herbicide that inhibits the growing point or meristem, such as an imidazolinone or a sulfonylurea. Exemplary genes in this category code for mutant ALS and AHAS enzyme as described, for example, by Lee et al., EMBO J. 7: 1241 (1988), and Miki et al., Theor. Appl. Genet. 80: 449 (1990), respectively. See also, U.S. Pat. Nos. 5,605,011; 5,013,659; 5,141,870; 5,767,361; 5,731,180; 5,304,732; 4,761,373; 5,331,107; 5,928,937. U.S. Pat. No. 4,975,374 to Goodman et al. disclose nucleotide sequences of glutamine synthetase genes which confer tolerance to herbicides such as L-phosphinothricin. The nucleotide sequence of a phosphinothricin-acetyl-transferase gene is provided in European Patent No. 0 242 246 and 0 242 236 to Leemans et al. De Greef et al., Bio/Technology 7: 61 (1989), describe the production of transgenic plants that express chimeric bar genes coding for phosphinothricin acetyl transferase activity. See also, U.S. Pat. Nos. 5,561,236; 5,648,477; 5,646,024. Exemplary genes conferring resistance to phenoxy proprionic acids and cyclohexones, such as sethoxydim and haloxyfop, are the Acc1-S1, Acc1-S2 and Acc1-S3 genes described by Marshall et al., Theor. Appl. Genet. 83: 435 (1992). An herbicide that inhibits photosynthesis, includes triazine (psbA and gs+ genes) and a benzonitrile (nitrilase gene). Przibilla et al., Plant Cell 3: 169 (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441 and 67442. Cloning and expression of DNA coding for a glutathione S-transferase is described by Hayes et al., Biochem. J. 285: 173 (1992). Acetohydroxy acid synthase, has been found to make plants that express this enzyme resistant to multiple types of herbicides, and has been introduced into a variety of plants (see, e.g., Hattori et al. (1995) Mol Gen Genet 246:419). Other genes that confer tolerance to herbicides include: a gene encoding a chimeric protein of rat cytochrome P4507A1 and yeast NADPH-cytochrome P450 oxidoreductase (Shiota et al. (1994) Plant Physiol 106:17), genes for glutathione reductase and superoxide dismutase (Aono et al. (1995) Plant Cell Physiol 36:1687, and genes for various phosphotransferases (Datta et al. (1992) Plant Mol Biol 20:619). Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are tolerant to these herbicides are described in U.S. Pat. Nos. 6,288,306 B1; 6,282,837 B1; and 5,767,373.

The methods here may be used with controlling expression of any desired plant product and is in an embodiment useful with controlling expression of a gene product. By way of example without limitation, a skilled person appreciates that among the various genes that may be used in the process are those that confer resistance to insects or disease, stress or fungi are among the myriad of example. Extensive discussions of such genes and their uses include, for example U.S. Pat. No. 9,637,736. By way of example without limitation, the gene may produce a product that provides a beneficial agronomic benefit such as herbicide tolerance, insect control, modified yield, fungal disease resistance, virus resistance, nematode resistance, bacterial disease resistance, plant growth and development, starch production, modified oils production, high oil production, modified fatty acid content, high protein production, fruit ripening, enhanced animal and human nutrition, biopolymers, environmental stress resistance, pharmaceutical peptides and secretable peptides, improved processing traits, improved digestibility, enzyme production, flavor, nitrogen fixation, hybrid seed production, fiber production, and biofuel production. Examples of genes of agronomic interest include those for herbicide resistance (U.S. Pat. Nos. 6,803,501; 6,448,476; 6,248,876; 6,225,114; 6,107,549; 5,866,775; 5,804,425; 5,633,435; and 5,463,175), increased yield (U.S. Pat. Nos. RE38,446; 6,716,474; 6,663,906; 6,476,295; 6,441,277; 6,423,828; 6,399,330; 6,372,211; 6,235,971; 6,222,098; and 5,716,837), insect control (U.S. Pat. Nos. 6,809,078; 6,713,063; 6,686,452; 6,657,046; 6,645,497; 6,642,030; 6,639,054; 6,620,988; 6,593,293; 6,555,655; 6,538,109; 6,537,756; 6,521,442; 6,501,009; 6,468,523; 6,326,351; 6,313,378; 6,284,949; 6,281,016; 6,248,536; 6,242,241; 6,221,649; 6,177,615; 6,156,573; 6,153,814; 6,110,464; 6,093,695; 6,063,756; 6,063,597; 6,023,013; 5,959,091; 5,942,664; 5,942,658, 5,880,275; 5,763,245; and 5,763,241), fungal disease resistance (U.S. Pat. Nos. 6,653,280; 6,573,361; 6,506,962; 6,316,407; 6,215,048; 5,516,671; 5,773,696; 6,121,436; 6,316,407; and 6,506,962), virus resistance (U.S. Pat. Nos. 6,617,496; 6,608,241; 6,015,940; 6,013,864; 5,850,023; and 5,304,730), nematode resistance (U.S. Pat. No. 6,228,992), bacterial disease resistance (U.S. Pat. No. 5,516,671), plant growth and development (U.S. Pat. Nos. 6,723,897 and 6,518,488), starch production (U.S. Pat. Nos. 6,538,181; 6,538,179; 6,538,178; 5,750,876; 6,476,295), modified oils production (U.S. Pat. Nos. 6,444,876; 6,426,447; and 6,380,462), high oil production (U.S. Pat. Nos. 6,495,739; 5,608,149; 6,483,008; and 6,476,295), modified fatty acid content (U.S. Pat. Nos. 6,828,475; 6,822,141; 6,770,465; 6,706,950; 6,660,849; 6,596,538; 6,589,767; 6,537,750; 6,489,461; and 6,459,018), high protein production (U.S. Pat. No. 6,380,466), fruit ripening (U.S. Pat. No. 5,512,466), enhanced animal and human nutrition (U.S. Pat. Nos. 6,723,837; 6,653,530; 6,5412,59; 5,985,605; and 6,171,640), biopolymers (U.S. Pat. Nos. RE37,543; 6,228,623; and U.S. Pat. Nos. 5,958,745, and 6,946,588), environmental stress resistance (U.S. Pat. No. 6,072,103), pharmaceutical peptides and secretable peptides (U.S. Pat. Nos. 6,812,379; 6,774,283; 6,140,075; and 6,080,560), improved processing traits (U.S. Pat. No. 6,476,295), improved digestibility (U.S. Pat. No. 6,531,648) low raffinose (U.S. Pat. No. 6,166,292), industrial enzyme production (U.S. Pat. No. 5,543,576), improved flavor (U.S. Pat. No. 6,011,199), nitrogen fixation (U.S. Pat. No. 5,229,114), hybrid seed production (U.S. Pat. No. 5,689,041), fiber production (U.S. Pat. Nos. 6,576,818; 6,271,443; 5,981,834; and 5,869,720) and biofuel production (U.S. Pat. No. 5,998,700). A gene of agronomic interest can affect the above mentioned plant characteristic or phenotype by encoding a RNA molecule that causes the targeted modulation of gene expression of an endogenous gene, for example via antisense (see e.g. U.S. Pat. No. 5,107,065); inhibitory RNA (“RNAi”, including modulation of gene expression via miRNA-, siRNA-, trans-acting siRNA-, and phased sRNA-mediated mechanisms, e.g. as described in published applications US 2006/0200878 and US 2008/0066206, and in U.S. patent application Ser. No. 11/974,469); or cosuppression-mediated mechanisms. The RNA could also be a catalytic RNA molecule (e.g. a ribozyme or a riboswitch; see e.g. US 2006/0200878) engineered to cleave a desired endogenous mRNA product. Thus, any transcribable polynucleotide molecule that encodes a transcribed RNA molecule of interest may be useful.

The term plant or plant material or plant part is used broadly herein to include any plant at any stage of development, or to part of a plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or aggregate of cells such as a friable callus, or a cultured cell, or can be part of a higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered a plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like. “Seed” refers to any plant structure that is formed by continued differentiation of the ovule of the plant, following its normal maturation point at flower opening, irrespective of whether it is formed in the presence or absence of fertilization and irrespective of whether or not the seed structure is fertile or infertile.

As referred to herein, “genomic sequence” refers to DNA within a genome that harbors that information required to produce a functional RNA or protein. Genomic sequence can comprise 5′ UTRs, 3′ UTRs, exons, and introns.

As referred to herein, “genomic locus” refers to a specific location or region within a genome. A genomic locus can comprise a location or region that contains non-coding DNA positioned between genes (intergenic). A genomic locus can comprise a location or region that contains coding DNA (genic). A genomic locus can comprise a location or region that contains both coding and non-coding DNA (genic and intergenic). A genomic locus can comprise location or region within a DNA sequence that has transcriptional activity. A genomic locus can comprise a location or region nearby a DNA sequence that has transcriptional activity.

As referred to herein, “coding sequence” or “CDS” refers to DNA that harbors the necessary information that is required to produce a functional RNA or protein. Coding sequence or CDS can include a DNA sequence starting with ATG and ending with a stop codon. The coding sequence or CDS usually does not contain introns, if no introns are required to produce the functional RNA or protein. Coding sequence, as referred to herein, excludes promoter elements.

As referred to herein, “plant” refers to any plant and includes monocots and dicots. A preferred embodiment provides the plant is a crop plant. Examples of a crop plants include soybean, wheat, alfalfa, potato, rice, corn, millet, barley, tomato, apple, pear, strawberry, orange, watermelon, pepper, carrot, sugar beets, yam, lettuce, spinach, sunflower, and rape seed. The plant can be a monocot or a dicot. Examples of monocots include, but are not limited to, oil palm, sugarcane, banana, Sudan grass, corn, wheat, rye, barley, oat, rice, millet and sorghum. Examples of dicots include, but are not limited to, safflower, alfalfa, soybean, coffee, amaranth, rapeseed, peanut, and sunflower. Orders of dicots include Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salcicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Camapnulales, Rubiales, Dipsacales, and Asterales. Genera of dicots include Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Galucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapsis, Solanum, Theobroma, Trifolium, Trigonella, Vicia, Vinca, Vilis, and Vigna. Orders of monocots include Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales. Genera of monocots include Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, and Zea. Other plants include Gymnospermae, such as the orders Pinales, Ginkgoales, Cycadales, and Gnetales, such as the genera Abies, Cunninghamia, Picea, Pinus, and Pseudotsuga, such as fir and pine. Modification of a nucleotide or amino acid sequences means a change to the sequence. One means of modification is mutagenesis. “Mutagenesis” as used herein refers to processes in which mutations are introduced into a selected DNA sequence. Mutations induced by endonucleases generally are obtained by a double-strand break, which results in insertion/deletion mutations (“indels”) that can be detected by deep-sequencing analysis. Such mutations typically are deletions of several base pairs, and have the effect of inactivating the mutated allele. In the methods described herein, for example, mutagenesis occurs via double-stranded DNA breaks made by nucleases targeted to selected DNA sequences in a plant cell. Such mutagenesis results in “nuclease-induced mutations” (e.g., nuclease-induced knockouts, such as TALE-nuclease-induced knockouts) and reduced expression of the targeted gene. Following mutagenesis, plants can be regenerated from the treated cells using known techniques (e.g., planting seeds in accordance with conventional growing procedures, followed by self-pollination).

As used herein, the term “altering the expression of” or “controlling expression of” refers to a process of changing the expression of a certain gene within a plant genome. The change in expression can be measured, for example, by using standard RNA or protein quantification assays. The change in expression can be relative to the expression within a wild type plant. The change in expression can result in differences in the expression level, timing or location.

The term “expression” as used herein refers to the transcription of a particular nucleic acid sequence to produce sense or antisense RNA or mRNA, and/or the translation of an mRNA molecule to produce RNA or a polypeptide, with or without subsequent post-translational events.

The term “modulating” as used herein refers to increasing or decreasing translational efficiency of an mRNA. This can be accomplished by inserting, removing, or altering a 5′ UTR sequence, a 3′ UTR sequence, or 5′ and 3′ UTR sequences.

A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes one or more expression control sequences, and an “expression control sequence” is a DNA sequence that controls and regulates the transcription and/or translation of another DNA sequence. Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The terms “regulatory region,” “control element,” and “expression control sequence” refer to nucleotide sequences that influence transcription or translation initiation and rate, and stability and/or mobility of the transcript or polypeptide product. Regulatory regions include, without limitation, promoter sequences, enhancer sequences, response elements, protein recognition sites, inducible elements, promoter control elements, protein binding sequences, 5′ and 3′ UTRs, transcriptional start sites, termination sequences, polyadenylation sequences, introns, and other regulatory regions that can reside within coding sequences, such as secretory signals, Nuclear Localization Sequences (NLS) and protease cleavage sites.

As used herein, “operably linked” means incorporated into a genetic construct so that expression control sequences effectively control expression of a coding sequence of interest. A coding sequence is “operably linked” and “under the control” of expression control sequences in a cell when RNA polymerase is able to transcribe the coding sequence into RNA, which if an mRNA, then can be translated. Thus, a regulatory region can modulate, e.g., regulate, facilitate or drive, transcription in the plant cell, plant, or plant tissue in which it is desired to express a modified target nucleic acid.

As used herein, “different genomic locus” refers to a location within the genome that is in a different location than the referenced sequence. The different genomic locus can be sequence on a different chromosome as the referenced sequence. The different genomic locus can be sequence on the same chromosome as the referenced sequence. If the different genomic locus is on the same chromosome as the referenced location, then the different genomic locus must not capture the transcriptional activity of the promoter from the referenced sequence.

A promoter is an expression control sequence composed of a region of a DNA molecule, typically upstream of the point at which transcription starts (generally near the initiation site for RNA polymerase II). Promoters are involved in recognition and binding of RNA polymerase and other proteins to initiate and modulate transcription. A promoter typically comprises at least a core (basal) promoter. A promoter also may include at least one control element such as an upstream element. Such elements include upstream activation regions (UARs) and, optionally, other DNA sequences that affect transcription of a polynucleotide such as a synthetic upstream element.

The choice of promoters useful in the methods depends upon the type of desired expression to be achieved. Factors, including, but not limited to, efficiency, selectability, inducibility, desired expression level, and cell or tissue specificity. For example, tissue-, organ- and cell-preferred promoters that confer transcription only or predominantly in a particular tissue, organ, and cell type, respectively, can be used. In some embodiments, promoters specific to vegetative tissues such as the stem, parenchyma, ground meristem, vascular bundle, cambium, phloem, cortex, shoot apical meristem, lateral shoot meristem, root apical meristem, lateral root meristem, leaf primordium, leaf mesophyll, or leaf epidermis can be suitable regulatory regions. In some embodiments, promoters that are essentially specific to seeds (“seed-preferential promoters”), that is, which are preferentially expressed to seed tissue, can be useful. Seed-specific promoters can promote transcription of an operably linked nucleic acid in endosperm and cotyledon tissue during seed development. Alternatively, constitutive promoters can promote transcription of an operably linked nucleic acid in most or all tissues of a plant, throughout plant development. Other classes of promoters include, but are not limited to, inducible promoters, such as promoters that confer transcription in response to inducers, such as external stimuli such as chemical agents, developmental stimuli, or environmental stimuli.

Constitutive promoters include, for example, ubiquitin (Christensen et al. (1989) Plant Mol. Biol. 12:619-632 and Christensen et al. (1992) Plant Mol. Biol. 18:675-689); the core promoter of the Rsyn7 promoter and other constitutive promoters disclosed in WO 99/43838 and U.S. Pat. No. 6,072,050; rice actin (McElroy et al. (1990) Plant Cell 2:163-171); pEMU (Last et al. (1991) Theor. Appl. Genet. 81:581-588); MAS (Velten et al. (1984) EMBO J. 3:2723-2730); maize histone promoter (Chaboute et al. Plant Molecular Biology, 8:179-191 (1987), Brignon et al., Plant Mol Bio 22(6):1007-1015 (1993); Rasco-Gaunt et al., Plant Cell Rep. 21(6):569-576 (2003)) and the like.

The promoter may be one which preferential expresses to particular tissue, organ or other part of a plant, or may express during a certain stage of development or under certain conditions. When referring to preferential expression, what is meant is expression at a higher level in the particular plant tissue than in other plant tissue.

The range of available promoters includes inducible promoters. An inducible regulatory element is one that is capable of directly or indirectly activating transcription of one or more DNA sequences or genes in response to an inducer. In the absence of an inducer the DNA sequences or genes will not be transcribed. Typically the protein factor that binds specifically to an inducible regulatory element to activate transcription is present in an inactive form which is then directly or indirectly converted to the active form by the inducer. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. Typically, the protein factor that binds specifically to an inducible regulatory element to activate transcription is present in an inactive form which is then directly or indirectly converted to the active form by the inducer. A plant cell containing an inducible regulatory element may be exposed to an inducer by externally applying the inducer to the cell or plant such as by spraying, watering, heating or similar methods. By way of example without limitation, the ERF inducible promoter is described. Other examples include the In2-1 and In2-2 gene from maize which respond to benzenesulfonamide herbicide safeners (U.S. Pat. No. 5,364,780; Hershey et al., Mol. Gen. Genetics 227: 229-237 (1991) and Gatz et al., Mol. Gen. Genetics 243: 32-38 (1994)) Tet repressor from Tn10 (Gatz et al., Mol. Gen. Genet. 227: 229-237 (1991); or the maize GST promoter, which is activated by hydrophobic electrophilic compounds that are used as pre-emergent herbicides; and the tobacco PR-la promoter, which is activated by salicylic acid.

In one embodiment a promoter of interest may have strong or weak transcriptional activity. A skilled person appreciates a promoter sequence can be modified to provide for a range of expression levels of and operably linked heterologous nucleic acid molecule. Generally, by “weak promoter” is intended a promoter that drives expression of a coding sequence at a low level. By “low level” is intended levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts. It is recognized that to increase transcription levels, enhancers can be utilized in combination with the promoter regions. Enhancers are nucleotide sequences that act to increase the expression of a promoter region. Enhancers are known in the art and include the SV40 enhancer region, the 35S enhancer element, and the like.

Non-limiting examples of promoters of interest can include constitutively expressed promoters such as the cauliflower mosaic virus (CaMV) 35S transcription initiation region and maize ubiquitin-1 promoter, fruit-specific promoters such as the ACC-oxidase (Barry, Plant J. 9:525-535, 1996) and E8 promoters (Mehta, Nat. Biotechnol. 20:613-618, 2011), seed-specific promoters such as the HaG3-A (Bogue, Mol. Gen. Genet. 222:49-57, 1990) and Psl (de Pater, Plant J. 6:133-140, 1994) promoters, floral tissue-specific promoters such as the END1 (Gömez, Planta 219:967-981, 2004) and TomA108 (Xu, Plant Cell Rep. 25:231-240, 2006) promoters, root-specific promoters such as the B33 (Farran, Transgenic Res. 11:337-346, 2002) and RB7 (Vaughan, J. Exp. Botany 57:3901-3910, 2006) promoters, the 1′ or 2′ promoters derived from T-DNA of Agrobacterium tumefaciens, promoters from a maize leaf-specific gene described by Busk (Plant J. 11:1285-1295, 1997), kn1-related genes from maize and other species, and chemical-inducible promoters such as the XVE (Zuo et al., The Plant Journal 24:265-273, 2000) and GVG (Aoyama and Chua, The Plant Journal 11:605-612, 1997) promoter systems.

A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA message stability or translation attenuation. Examples of 3′ UTRs include, without limitation, polyadenylation signals and transcription termination sequences. A polyadenylation region at the 3′-end of a coding region can also be operably linked to a coding sequence. The polyadenylation region can be derived from the natural gene, from various other plant genes, or from an Agrobacterium T-DNA.

As used herein, the amino acid sequences follow the standard single letter or three letter nomenclature. All protein or peptide sequences are shown in conventional format where the N-terminus appears on the left and the carboxyl group at the C-terminus on the right. Amino acid nomenclature, both single letter and three letter, for naturally occurring amino acids are as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), leucine (Leu; L), isoleucine (Ile; I), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

As used herein, the term “uncharged polar” amino acids include glycine, serine, threonine, cysteine, tyrosine, asparagine and glutamine. The term “nonpolar” amino acids include alanine, valine, leucine, isoleucine, proline, phenylalanine, tryptophan, and methionine. The term “charged polar” amino acids includes aspartic acid, glutamic acid, lysine, arginine and histidine.

As used herein, the term “nucleic acid” refers to a polymer made up of nucleotide monomers. A nucleic acid can be single stranded or double stranded, and can be linear or circular. Where single-stranded, a nucleic acid can be a sense strand or an antisense strand. A nucleic acid can be composed of DNA (e.g., cDNA, genomic DNA, synthetic DNA, or a combination thereof), RNA, or DNA and RNA. Further, nucleic acids can contain information for gene expression, including, but not limited to, promoters, 5′ UTRs, 3′ UTRs, coding sequences, and terminators.

As used herein, deoxyribonucleic acid (DNA) is a biopolymer that comprises four nucleotides linked together by phosphodiester bridges. The four nucleotides include dAMP (2′-deoxyadenosine-5-monophosphate), dGMP (2′-deoxyguanosine-5-monophosphate), dCMP (2′-deoxycytosine-5-monophosphate) and dTMP (2′-deoxythymosine-5-monophosphate).

As used herein, the term “codon” refers to nucleotide triplets which code for amino acids, Due to the redundancy of the genetic code, the same amino acid can be coded for by different codons. The following is a list of amino acids and their respective codons: Met (ATG); Glu (GAA, GAG); Val (GTA, GTC, GTG, GTT); Arg (CGA, CGC, CGG, CGT, AGA, AGG); Leu (CTA, CTC, CTG, CTT, TTA, TTG); Ser (TCA, TCC, TCG, TCT, AGC, AGT); Thr (ACA, ACC, ACG, ACT); Pro (CCA, CCC, CCG, CCT); Ala (GCT, GCA, GCC, GCG); Gly (GGA, GGC, GGG, GGT); Ile (ATA, ATC, ATT); Lys (AAA, AAG); Asn (AAC, AAT); Gin (CAG, CAA); His (CAC, CAT); Asp (GAC, GAT); Tyr (TAC, TAT); Cys (TGC, TGT); Phe (TTC, TTT); and Trp (UGG)

One means of determining the percent sequence identity between a particular nucleic acid or amino acid sequence and a sequence referenced by a particular sequence identification number is as follows. First, a nucleic acid or amino acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -l; -r is set to 2; and all other options are left at their default setting. For example, the following command can be used to generate an output file containing a comparison between two sequences: C:\B12seq c:\seq1.txt -j c:\seq2.txt -p blastn -o c:\output.txt -q -1 -r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence (e.g., SEQ ID NO:1), or by an articulated length (e.g., 100 consecutive nucleotides or amino acid residues from a sequence set forth in an identified sequence), followed by multiplying the resulting value by 100. For example, a nucleic acid sequence that has 8,000 matches when aligned with the sequence set forth in SEQ ID NO:1 is 97.3 percent identical to the sequence set forth in SEQ ID NO:3 (i.e., 8,000±8,218×100=97.3). It is noted that the percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. It also is noted that the length value will always be an integer.

Identity to a sequence described would mean a sequence having at least 65% sequence identity, more preferably at least 70% sequence identity, more preferably at least 75% sequence identity, more preferably at least 80% identity, more preferably at least 85% 86%, 87%, 88%, 89%, 90%, 91%, 92%, 93%, 94%, 95%, 96%, 97%, 98%, 99% sequence identity.

When referring to hybridization techniques, all or part of a known nucleotide sequence can be used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the DNA sequences. Methods for preparation of probes for hybridization and for construction of cDNA and genomic libraries are generally known in the art and are disclosed (Sambrook et al., 2001).

For example, the sequences useful here, or one or more portions thereof, may be used as a probe capable of specifically hybridizing to corresponding sequences. To achieve specific hybridization under a variety of conditions, such probes include sequences that are unique among the sequences to be screened and are preferably at least about 10 nucleotides in length, and most preferably at least about 20 nucleotides in length. Such sequences may alternatively be used to amplify corresponding sequences from a chosen plant by PCR. This technique may be used to isolate sequences from a desired plant or as a diagnostic assay to determine the presence of sequences in a plant. Hybridization techniques include hybridization screening of DNA libraries plated as either plaques or colonies (Sambrook et al., 2001) Molecular Cloning: A Laboratory Manual (3rd ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 50° C. Exemplary high stringency conditions include hybridization in 50% formamide, 1 M NaCl, 0.1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-284 (1984): Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of the complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with 90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (2001) Molecular Cloning: A Laboratory Manual (3rd ed., Cold Spring Harbor Laboratory Press, Plainview, N.Y.) and Haymes et al. (1985) In: Nucleic Acid Hybridization, a Practical Approach, IRL Press, Washington, D.C.

In general, sequences that correspond to the nucleotide sequences described and hybridize to the nucleotide sequence disclosed herein will be at least 50% homologous, 70% homologous, and even 85%, 86%, 87%, 88%, 89%, 90%, 91%, 92%, 93%, 94%, 95%, 96%, 97%, 98%, 99% homologous or more with the disclosed sequence. That is, the sequence similarity between probe and target may range, sharing at least about 50%, about 70%, and even about 85% or more sequence similarity.

The relevant sequences useful in the processes include “functional variants” of the sequence disclosed. Functional variants include, for example, sequences having one or more nucleotide substitutions, deletions or insertions and wherein the variant retains desired activity, Functional variants can be created by any of a number of methods available to one skilled in the art, such as by site-directed mutagenesis, induced mutation, identified as allelic variants, cleaving through use of restriction enzymes, or the like. Activity can likewise be measured by any variety of techniques, including measurement of reporter activity as is described at U.S. Pat. No. 6,844,484, Northern blot analysis, or similar techniques. The '484 patent describes the identification of functional variants of different promoters.

The invention further encompasses a “functional fragment,” that is, a sequence fragment formed by one or more deletions from a larger regulatory element. Such fragments should retain the desired activity. Activity can be measured by Northern blot analysis, reporter activity measurements when using transcriptional fusions, and the like. See, for example, Sambrook et al. (2001). Functional fragments can be obtained by use of restriction enzymes to cleave the naturally occurring nucleotide sequences; by synthesizing a nucleotide sequence from the naturally occurring DNA sequence; or can be obtained through the use of PCR technology See particularly, Mullis et al. (1987) Methods Enzymol. 155:335-350) and Erlich, ed. (1989) PCR Technology (Stockton Press, New York).

Provided are methods for controlling expression of genes using endogenous plant promoters. In some embodiments, the method involves the insertion of selectable markers (e.g., Bar) downstream of endogenous plant promoters. In some embodiments, the method involves the insertion of screenable markers (e.g., YFP) downstream of endogenous plant promoters. In some embodiments, the method involves the insertion of endogenous plant genes (e.g., EPSPS) downstream of endogenous plant promoters. The methods in one embodiment provide plant cells, plant parts and plants that are glyphosate tolerant that contain a specific genetic modification. In one embodiment, the genetic modification is in the soybean genome at the GmUbi3 locus (Glyma20g27950) as shown in FIG. 5 and SEQ ID NO:16. In one embodiment, the genetic modification is in the soybean genome at the GmUbi3 locus as shown in SEQ ID NO:7. In one embodiment, the genetic modification is in the soybean genome at the GmUbi3 locus as shown in SEQ ID NO:8. In one embodiment, the genetic modification is in the soybean genome at the GmUbi3 locus as shown in SEQ ID NO:9. In one embodiment, the genetic modification is in the soybean genome at the GmUbi3 locus as shown in SEQ ID NO:10. In one embodiment, the genetic modification is in the soybean genome at the GmUbi3 locus as shown in SEQ ID NO:11. In one embodiment, the genetic modification is in the soybean genome at the GmUbi3 locus as shown in SEQ ID NO:12. In one embodiment, the genetic modification is in the soybean genome at the GmUbi3 locus as shown in SEQ ID NO:13. In one embodiment, the genetic modification is in the soybean genome at the GmUbi3 locus as shown in SEQ ID NO:14. In one embodiment, the genetic modification is in the soybean genome at the GmERF10 locus (Glyma17g15460) shown in SEQ ID NO:17. In one embodiment, the genetic modification is in the soybean genome at the GmERF10 locus as shown in SEQ ID NO:15.

This document also provides compositions of matter that include seeds with a specific genetic alteration. In some embodiments, a composition of matter can include plant cells, plant parts or plants modified with the methods as provided herein. The compositions of matter can be packaged using packaging material well known in the art to prepare a composition of matter. A composition of matter also can have a label (e.g., a tag or label secured to the packaging material, a label printed on the packaging material, or a label inserted within the package).

The invention will be further described in the following examples, which do not limit the scope of the invention described in the claims. All references cited herein are incorporated herein by reference in their entirety.

### Examples

### Example 1—Identifying a Soybean Promoter with a Desired Expression Profile for Expression of Gmmepsps

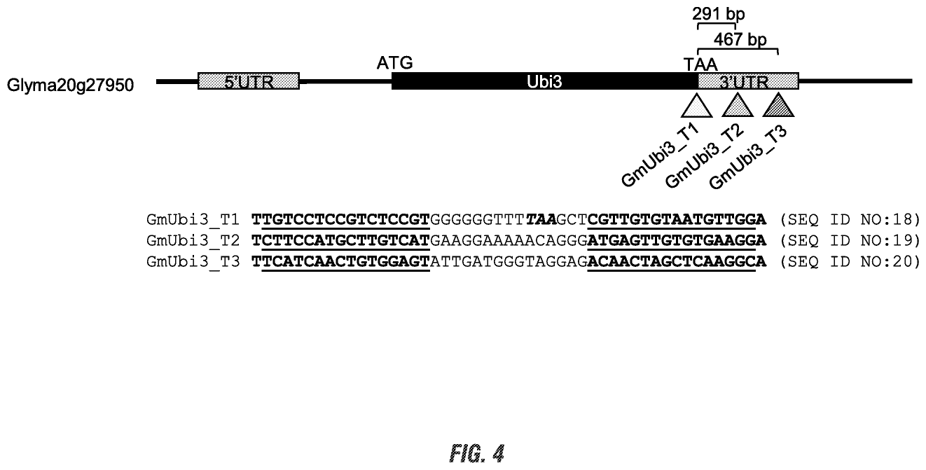

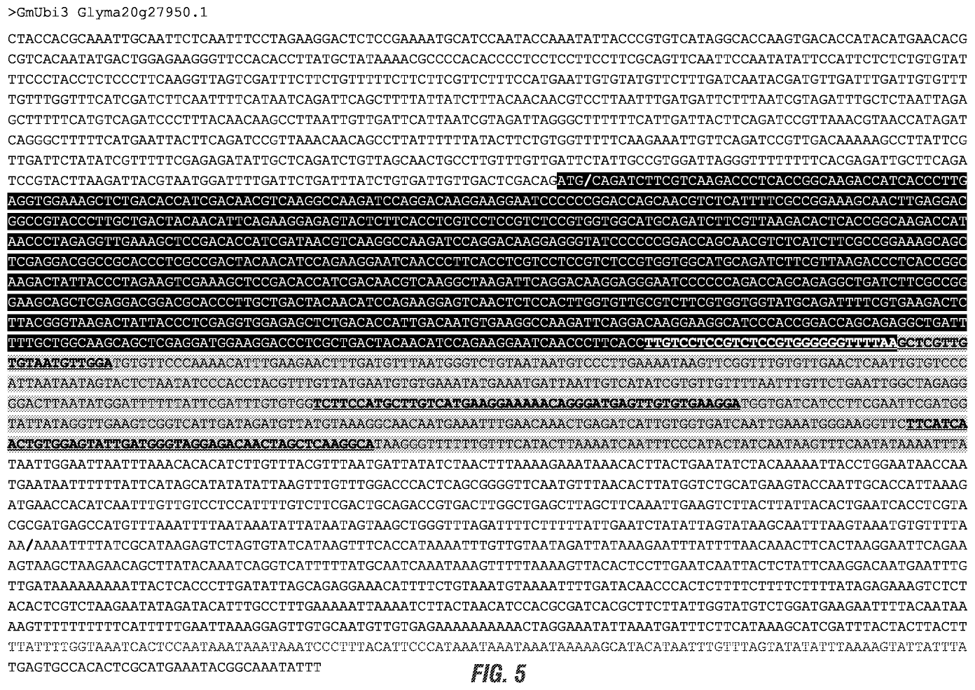

To achieve effective expression levels of a modified endogenous EPSPS gene within soybean (Gmmepsps) for conferring resistance to glyphosate, we sought to identify a promoter that has strong transcriptional activity in most tissue types at most developmental stages. To this end, we searched for soybean genes that were highly expressed in most cells during plant development. Here, we identified a candidate ubiquitin gene (GmUbi3) located on chromosome 20. GmUbi3 is a gene with one intron located within the 5′ UTR (FIGS. 4 and 5). GmUbi3 is highly expressed in different organs of soybean, with expression levels 7 fold higher than CaMV35S (Hernandez-Garcia et al., BMC Plant Biology 10:237, 2010).

### Example 2—Engineering Sequence-Specific Nucleases to Recognize and Cleave the GmUbi3 3′ UTR DNA Sequence

To capture the transcriptional activity of the GmUbi3 promoter, and to preserve the Ubi3 gene within the soybean genome, we chose to target Gmmepsps and reporter genes downstream of the GmUbi3 coding sequence (3′ insertion). Therefore, we designed three TALEN pairs targeting sequence near or downstream of the GmUbi3 stop codon. The TALEN pairs were designated as GmUbi3_T01.1, GmUbi3_T02.1 and GmUbi3_T03.1. The corresponding TALEN target sequences were designated as GmUbi3_T1, GmUbi3_T2 and GmUbi3_T3 (FIG. 4). TALENs were synthesized and cloned into bacterial vectors harboring the plant promoter, nopaline synthase (NOS). See TABLE 1 for the repeat variable diresidue (RVD) composition within the TALEN monomers for GmUbi3_T01.1, GmUbi3_T02.1 and GmUbi3_T03.1.

TABLE 1
GmUbi3 TALEN target sequences and RVD composition

| | | | SEQ |
|---|---|---|---|
| | | Target DNA | ID |
| | RVD composition | sequence | NO: |
| GmUbi3_T01.1 | NG-NN-NG-HD-HD- | TGTCCTCCGTCTCCGT | 19 |
| (Left) | NG-HD-HD-NN-NG- | | |
| | HD-NG-HD-HD-NN- | | |
| | NG | | |
| GmUbi3_T01.1 | HD-HD-NI-NI-HD- | CCAACATTACACAACT | 20 |
| (Right) | NI-NG-NG-NI-HD- | | |
| | NI-HD-NI-NI-HD-NG | | |
| GmUbi3_T02.1 | HD-NG-NG-HD-HD- | CTTCCATGCTTGTCAT | 21 |
| (Left) | NI-NG-NN-HD-NG- | | |
| | NG-NN-NG-HD-NI- | | |
| | NG | | |
| GmUbi3_T02.1 | HD-HD-NG-NG-HD- | CCTTCACACAACTCAT | 22 |
| (Right) | NI-HD-NI-HD-NI-NI- | | |
| | HD-NG-HD-NI-NG | | |
| GmUbi3_T03.1 | NG-HD-NI-NG-HD- | TCATCAACTGTGGAGT | 23 |
| (Left) | NI-NI-HD-NG-NN- | | |
| | NG-NN-NN-NI-NN- | | |
| | NG | | |
| GmUbi3_T03.1 | NN-HD-HD-NG-NG- | GCCTTGAGCTAGTTGT | 24 |
| (Right) | NN-NI-NN-HD-NG- | | |
| | NI-NN-NG-NG-NN- | | |
| | NG | | |

### Example 3—Testing GmUbi3 TALEN Activity in Soybean Protoplasts

To assess the activity of TALEN pairs GmUbi3_T01.1, GmUbi3_T02.1, and GmUbi3_T03.1, soybean protoplasts were transformed with 20 ug of each TALEN monomer plasmid. As a control for transformation efficiency, protoplasts were transformed with 20 ug of pCLS26487 (encoding YFP). For each sample, approximately 500,000 protoplasts were transformed by polyethylene glycol.

To determine transformation efficiency, soybean protoplasts transformed with pCLS26487 were checked for YFP expression ˜24 hours post transformation. The total number of YFP-positive cells were counted and divided by the total number of cells. From three independent fields, the transformation frequency was calculated to be 61.3%. This number was used to adjust the TALEN-induced NHEJ-mutation frequency as determined by 454 pyrosequencing.

To determine the mutation frequency associated with each TALEN pair, genomic DNA was isolated from protoplasts ˜48 hours post transformation, and amplicons encompassing the T1, T2, and T3 target sites were deep sequenced using 454 pyrosequencing. All three TALEN pairs were active, with varying ranges of mutation frequencies. GmUbi3_T01.1 was the least active, with mutation frequencies ˜0.9%. GmUbi3_T02.1 had mutation frequencies of ˜2.8%, and GmUbi3_T03.1 had mutation frequencies of ˜8.1% (TABLE 2)

TABLE 2
GmUbi3 TALEN activity in soybean protoplasts

| | | Normalized Mutation |
|---|---|---|
| TALEN pair | Mutation Frequency (%) | Frequency (%) |
| GmUbi3_T01.1 | 0.72 | 0.91 |
| GmUbi3_T02.1 | 1.70 | 2.76 |
| GmUbi3_T03.1 | 4.98 | 8.12 |

### Example 4—Donor Molecule Design for Targeted Insertion of Gmmepsps, BAR or YFP Downstream of GmUbi3

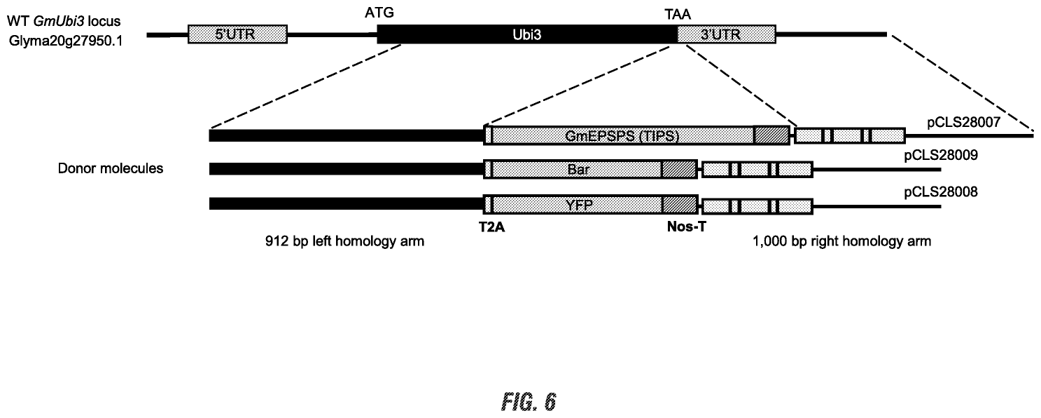

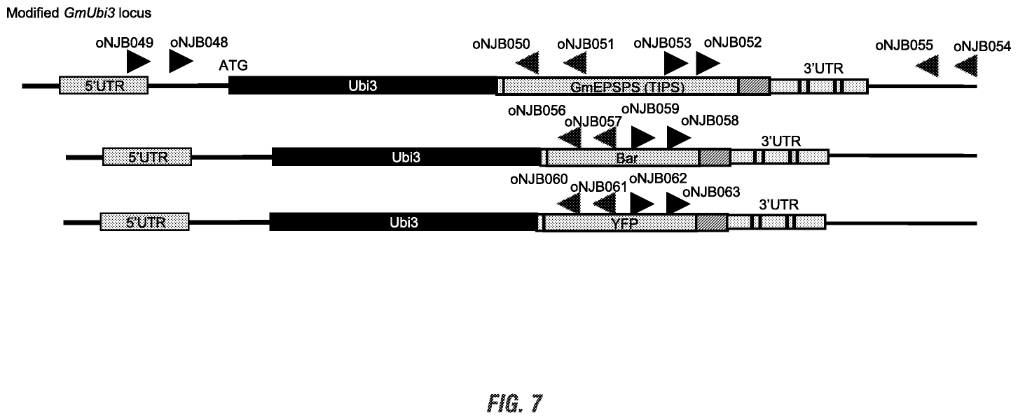

Donor molecules were designed to insert a promoterless YFP reporter into GmUbi3 (pCLS28008), as well as a promoterless Bar (pCLS28009) and modified GmEPSPS gene containing the TIPS mutations (pCLS28007) (FIG. 6). The left homology arms contained 912 bp of Ubi3 sequence, beginning with sequence immediately downstream of the start codon and ending with sequence immediately upstream of the stop codon. The left homology arm was fused to an in-frame T2A sequence followed by the YFP, Bar or mutant EPSPS sequence. The right homology arm contained 1000 nt of sequence from the Ubi3 3′ UTR and downstream region. Four SNPs were introduced into the left homology arm to prevent GmUbi3_T02.1 and GmUbi3_T03.1 from cleaving. These SNPs were introduced to change the T at the -l position to a C or G, thereby reducing the likelihood of TALEN binding. Sequences for donor molecule arms were taken from the reference genome sequence, which is homologous to the Bert Ubi3 sequence. The anticipated genetic modifications using each of the three donors is illustrated in FIG. 7.

### Example 5—Testing Targeted Insertion Frequencies in Soybean Cotyledons

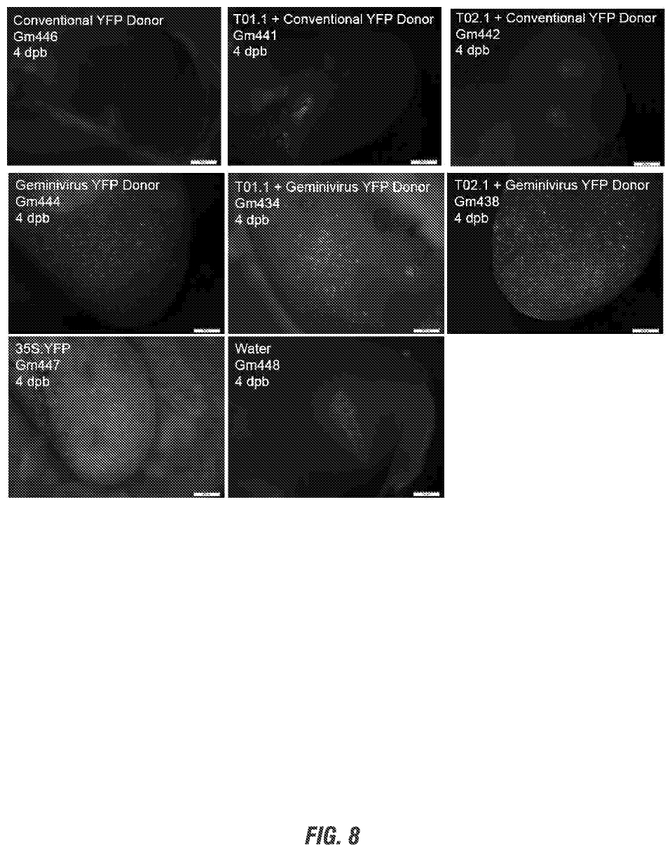

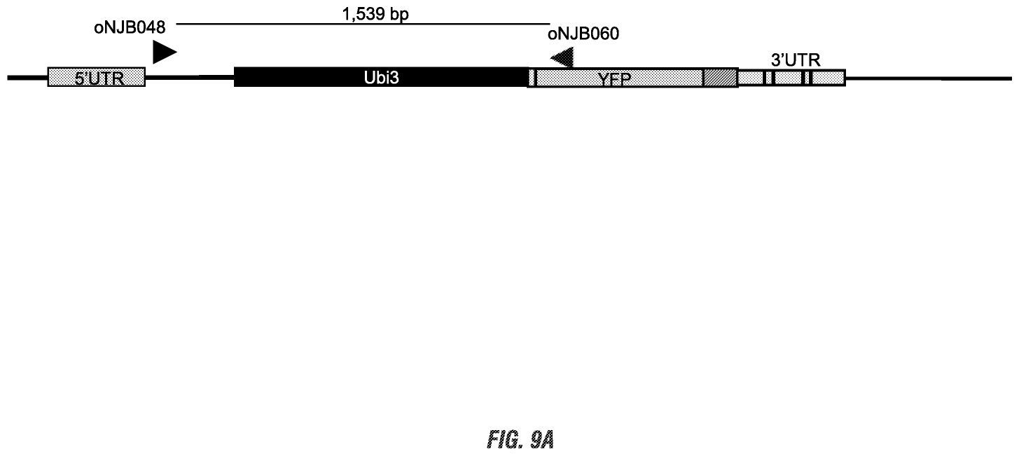

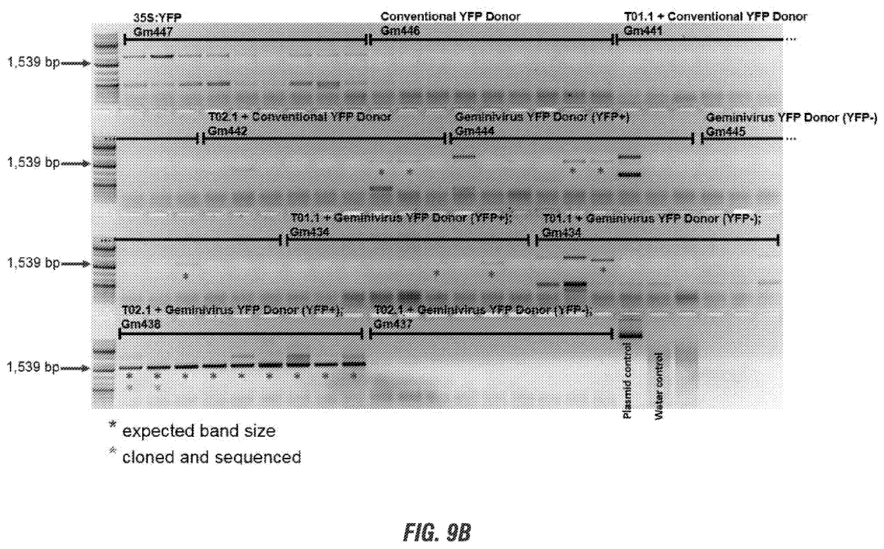

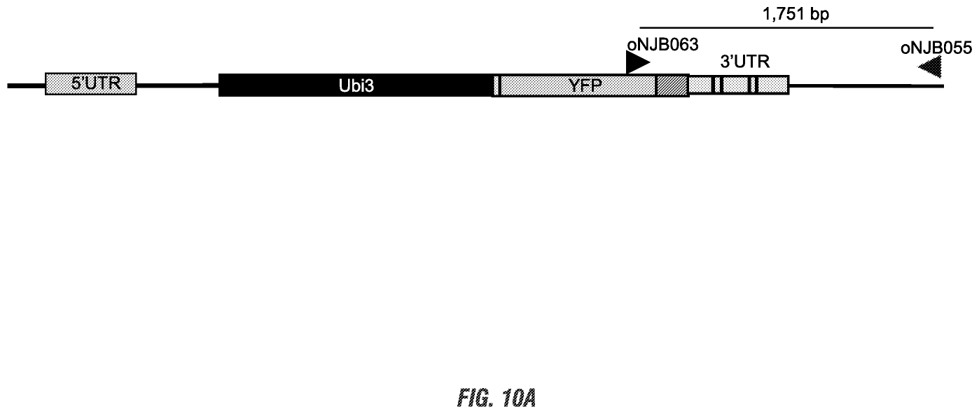

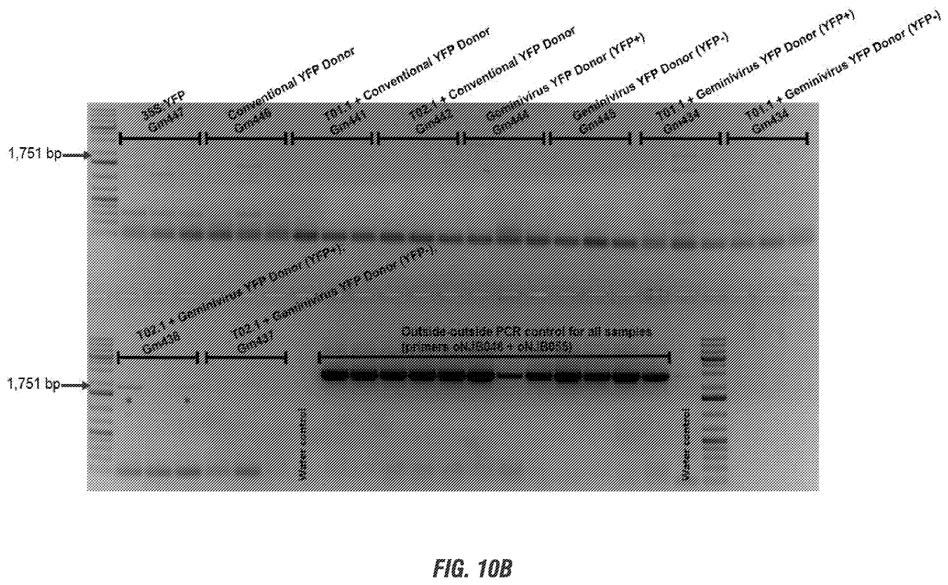

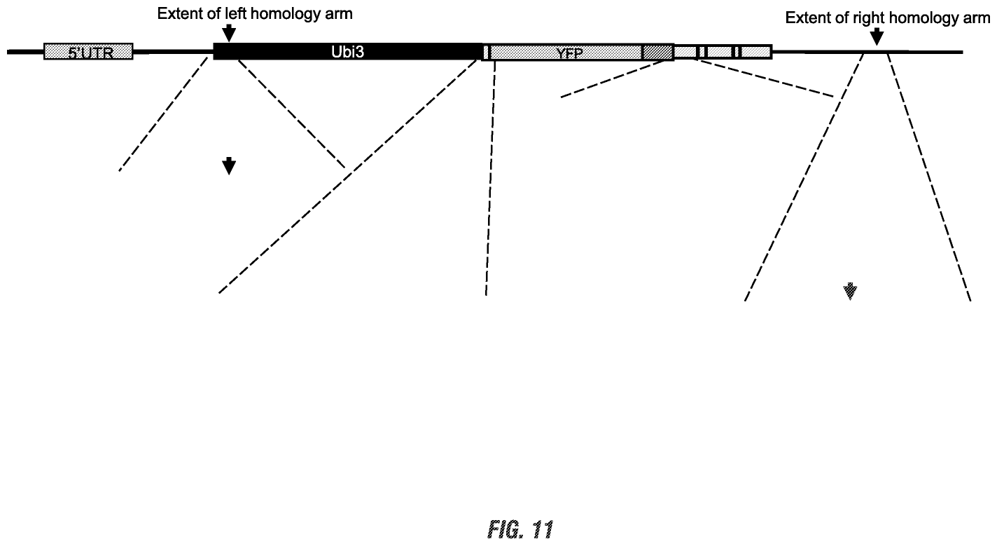

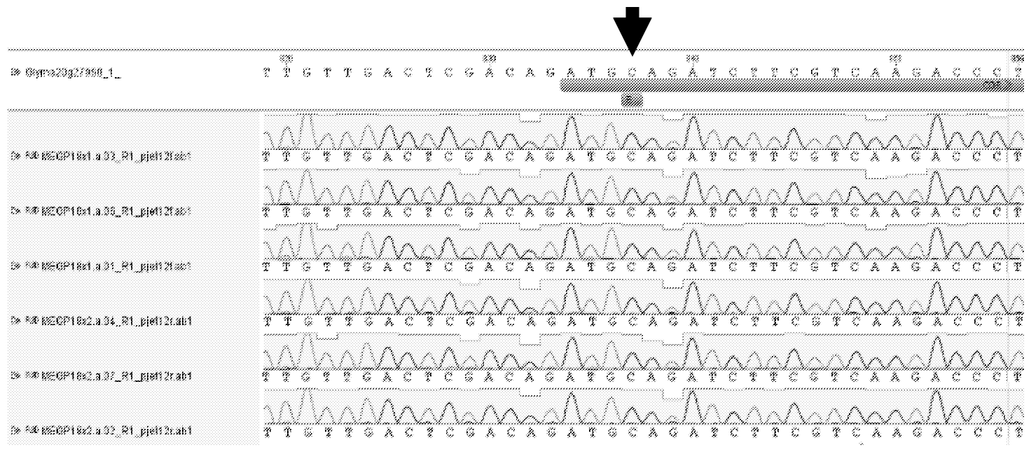

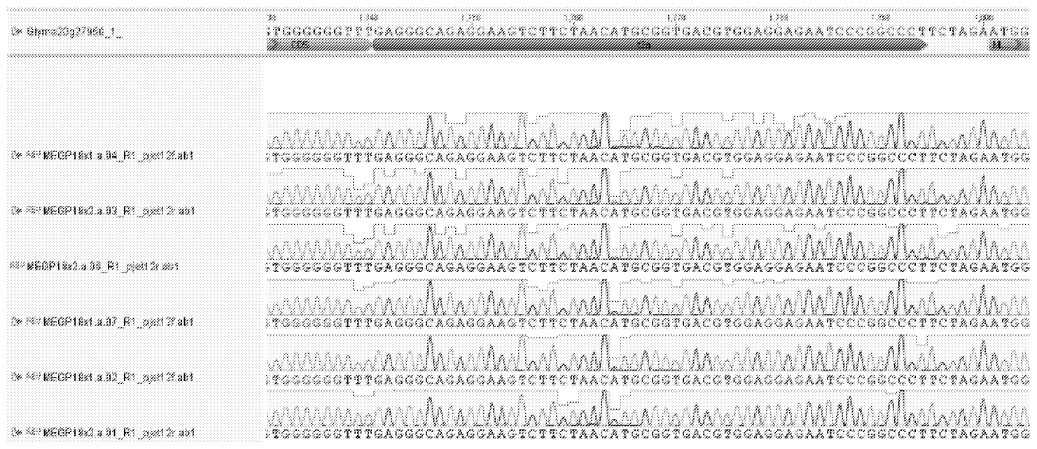

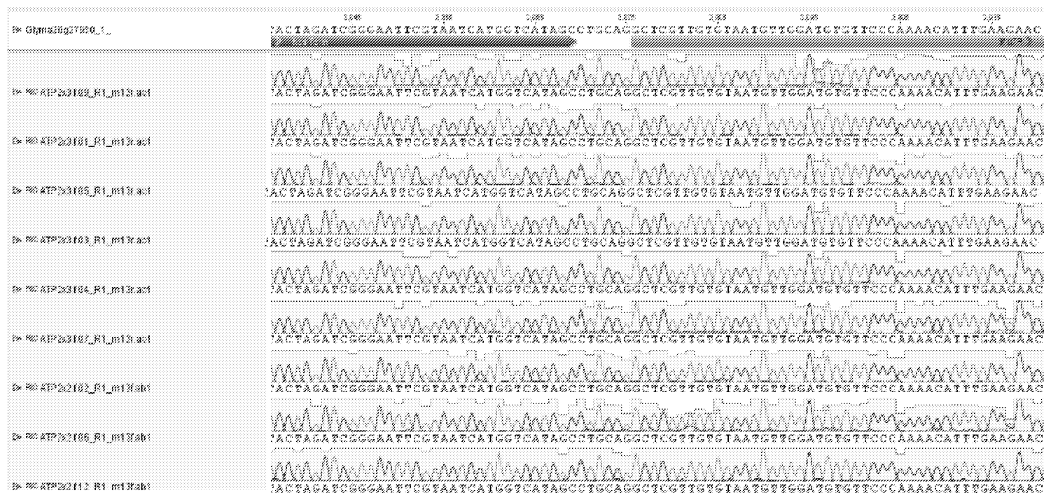

Next, we delivered our genome engineering reagents via biolistics to immature soybean cotyledons. Soybean cotyledons were bombarded with genome engineering reagents designed to insert YFP into Ubi3. To facilitate homologous recombination, we cloned the YFP donor molecule sequence into a Bean yellow dwarf virus replicon plasmid. Delivery of donor molecules on geminivirus replicons promotes homologous recombination, most likely through the amplification process of the replicon (Baltes et al., Plant Cell, 26:151-163, 2014; Cermak et al., Genome Biology, 16: 1-15, 2015). Herein, geminivirus donor molecules refers to donor molecules that have been cloned into geminivirus replicons, and conventional donor molecules refers to donor molecules that do not have additional viral amplification sequences. Cotyledons were bombarded with conventional and geminivirus donor molecules, along with TALEN pairs GmUbi3_T01.1 and GmUbi3_T02.1. Approximately four days post bombardment, we observed few to no cells expressing YFP in samples delivered conventional YFP donors; however, we observed large numbers of cells expressing YFP in samples delivered geminivirus YFP donors (FIG. 8). To verify that YFP expression was due to gene targeting, genomic DNA was extracted from cotyledons and subjected to 5′ and 3′ junction PCRs. Consistent with YFP expression, we observed very little to no amplification of sequence in samples delivered the conventional YFP donor; however, we observed efficient amplification of sequence with the expected size for both 5′ and 3′ junctions for samples delivered T02.1+geminivirus YFP donor (FIGS. 9 and 10). Cloning and sequencing of the products revealed precise homologous recombination for all clones (FIG. 11).

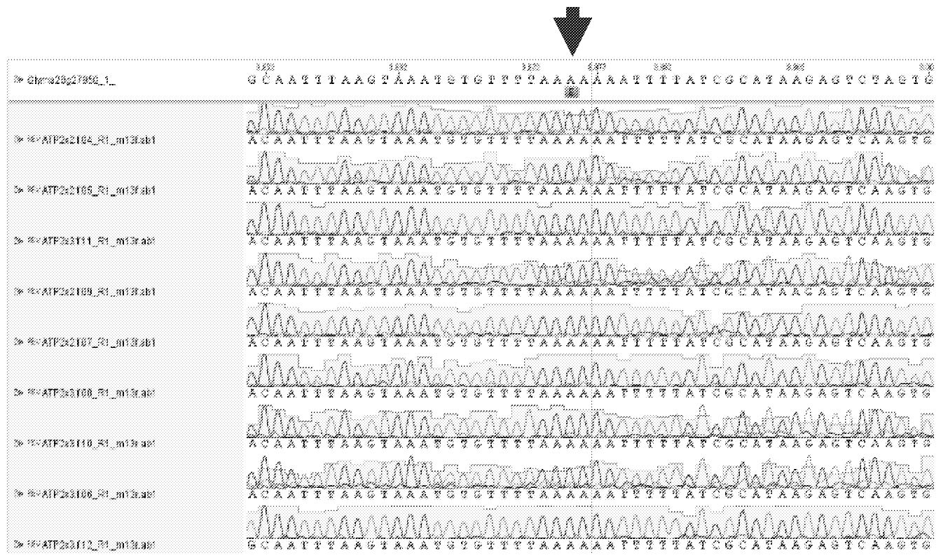

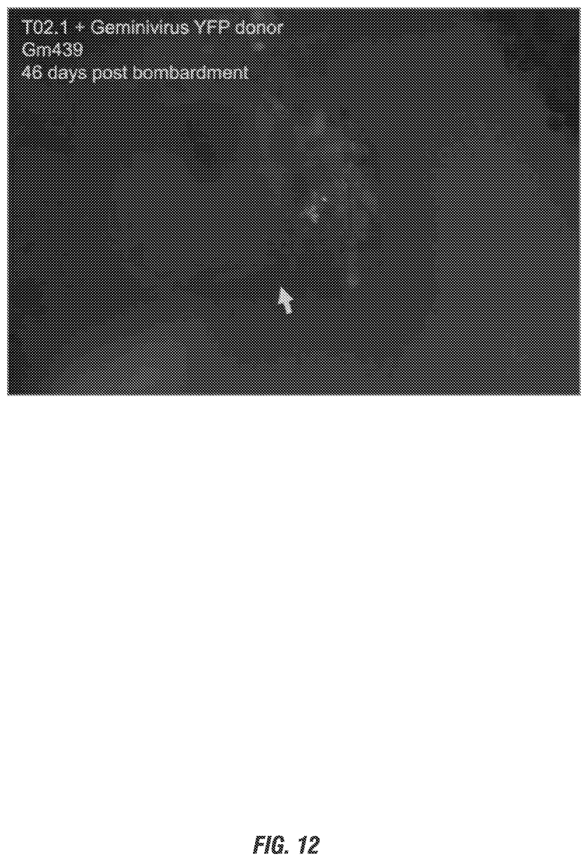

After demonstrating that our geminivirus reagents are efficient at promoting gene targeting in soybean cotyledons, we followed YFP expression over 46 days. We observed YFP-positive calli formation in samples delivered T02.1+geminivirus YFP donor 46 days post bombardment (FIG. 12). Notably, most YFP expression was lost a few weeks post bombardment after calli started to cover the bombarded tissue. However, this loss in YFP expression was also observed for the YFP controls.

### Example 6—Testing Targeted Insertion Frequencies in Soybean Protoplasts

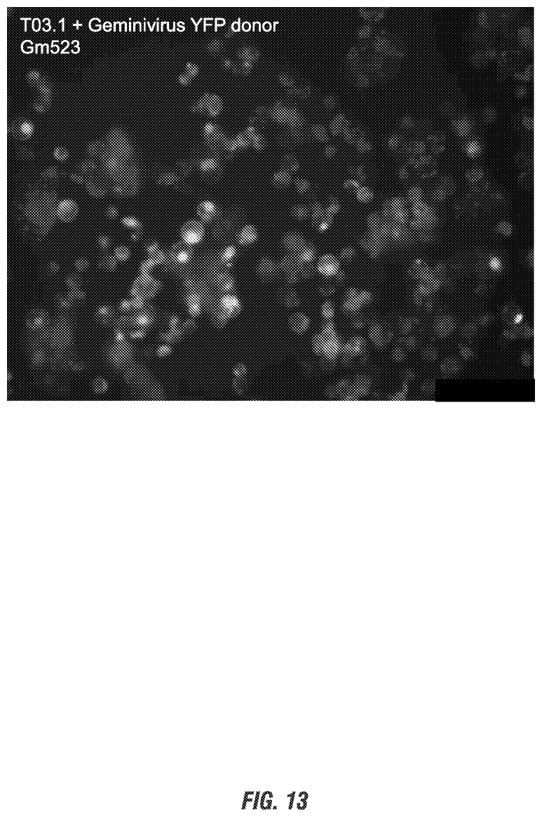

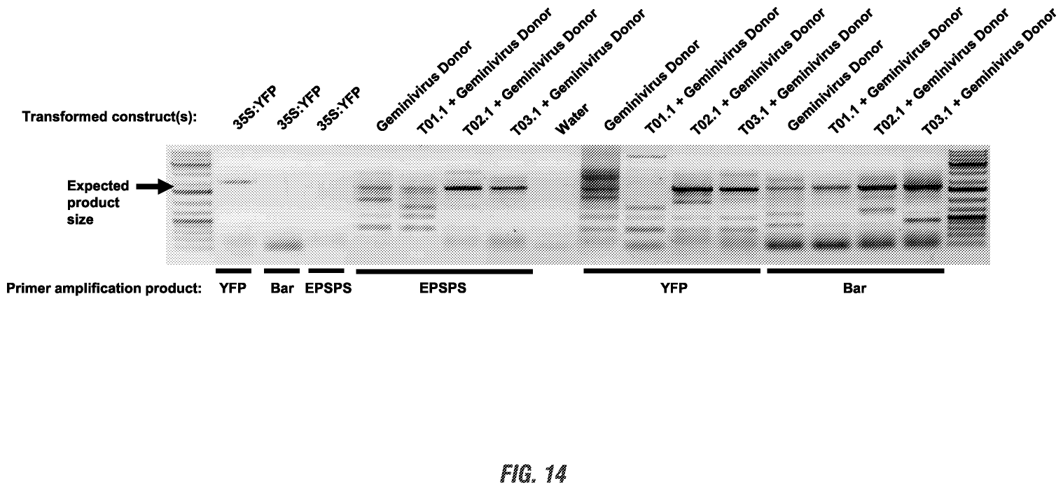

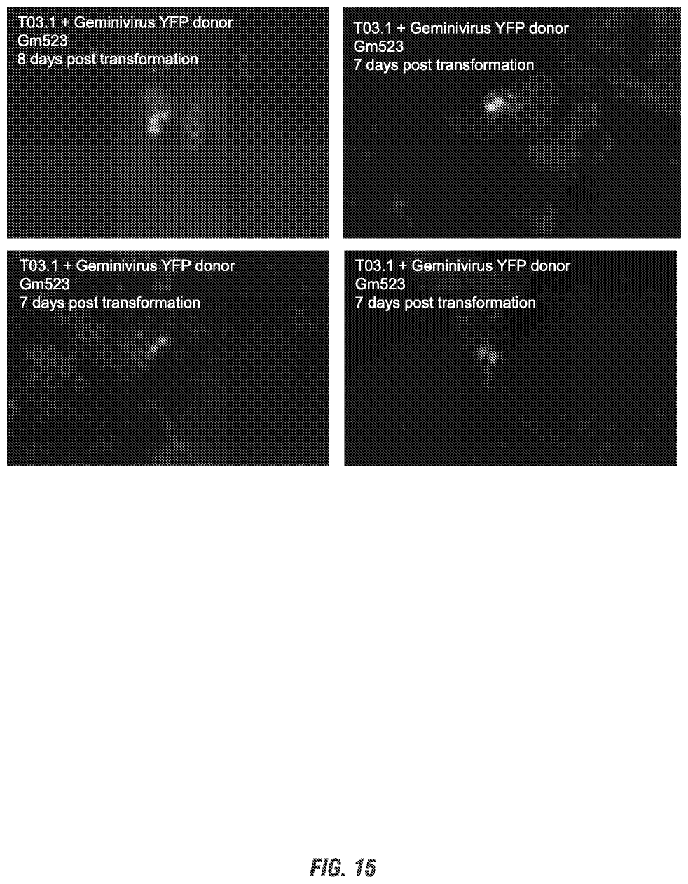

In addition to immature cotyledons, we also delivered our genome engineering reagents to soybean protoplasts. Here, cells were transformed via polyethylene glycol with plasmid encoding each TALEN pair, along with donor molecules (either conventional or geminivirus donor molecules). Similar to the results in cotyledons, we observed few cells expressing YFP after transformation with conventional YFP donors. However, we observed large numbers of cells expressing YFP after transformation with geminivirus donors (FIG. 13). Gene targeting was detected molecularly by extracting genomic DNA from protoplasts and performing a 5′ junction PCR (FIG. 14). In addition to confirming successful insertion of YFP into Ubi3, we also confirmed successful insertion of Bar and mutant GmEPSPS in to Ubi3 (FIG. 14). Protoplast transformed with GmUbi3_T03.1+geminivirus YFP donor were cultured in regeneration medium and monitored for two weeks. During this time, we observed evidence of elongation and division of YFP-positive cells (FIG. 15).

### Example 7—Generating Soybean Plants with Gmmepsps Integrated Downstream of GmUbi3

To generate soybean plants with Gmmepsps downstream of GmUbi3, sequence harboring the Gmmepsps donor molecule and sequence encoding the TALEN pair GmUbi3_T03.1 is stably integrated into the soybean genome using conventional transformation methods. Conventional transformation methods to integrate sequence within the soybean genome include biolistics (Rech et al., supra) Agrobacterium-mediated transformation (Yamada et al., supra), and protoplast regeneration (Dhir et al., Plant Physiology, 99: 81-88, 1992). Selectable markers are used to facilitate recovery of transgenic plants. Suitable selectable markers include bar, hygromycin and kanamycin.

To detect the targeted insertion of Gmmepsps chr 1 downstream of Ubi3, transgenic plants are first screened by PCR using primers designed to amplify the predicted 5′ and 3′ junction. T0 candidate plants are then allowed to self to produce T1 seeds and plants that are homozygous for the Gmmepsps chr 1 insertion sequence.

### Example 8—Assessing Glyphosate Tolerance in Soybean Plants with Gmmepsps Integrated Downstream of GmUbi3

Modified soybean plants are then tested for glyphosate tolerance by exposure to N-(phophonomethyl)glycine. Various means are available to a skilled person to test for tolerance. By way of example and without limitation, one method for assessing glyphosate tolerance include germination of seedlings on medium containing N-(phophonomethyl)glycine. Seeds are embedded within agar in plates and germinated. Agar plates are made with a dilution series of N-(phophonomethyl)glycine, ranging from a concentration 1 M to a concentration of 1 nM. Dilution increments are introduced by 10 fold decreases in N-(phophonomethyl)glycine concentration. Germinated seedlings containing the EPSPS knockin event are monitored for glyphosate tolerance, and compared to wild type seedlings. Sustained growth and rooting by the modified seedlings on medium containing N-(phophonomethyl)glycine indicates tolerance to the herbicide.

Another method for assessing glyphosate tolerance is by spraying soybean plants with a solution containing N-(phophonomethyl)glycine. Here, modified soybean plants are sprayed with a series of solutions containing concentrations of N-(phophonomethyl)glycine ranging from a 1 M to 1 nM. Dilution increments are introduced by 10 fold decreases in N-(phophonomethyl)glycine concentration from 1 M to 1 nM. Modified plants containing the EPSPS knockin event are monitored for glyphosate tolerance, and compared to wild type plants. Sustained or restarted growth after exposure to the herbicide indicates tolerance to the herbicide.

A still further method for assessing glyphosate tolerance is excision of plant parts followed by exposure to solutions containing N-(phophonomethyl)glycine. The plant parts from modified soybean plants are whole leaves. Whole leaves are excised from soybean plants and submerged in a solution a series of solutions containing concentrations of N-(phophonomethyl)glycine ranging from a 1 M to 1 nM. Dilution increments are introduced by 10 fold decreases in N-(phophonomethyl)glycine concentration from 1 M to 1 nM. Modified plants containing the EPSPS knockin event are monitored for glyphosate tolerance, and compared to wild type plants. Sustained or restarted growth after exposure to the herbicide indicates tolerance to the herbicide.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for generating an herbicide tolerant plant, the method comprising:
a. providing a plant cell comprising one or more endogenous 5-enolpyruvylshikimate-3-phosphate synthase (EPSPS) encoding genes;
b. inserting a modified EPSPS (mepsps) genomic sequence or coding sequence into a different genomic locus than the locus of said one or more endogenous EPSPS encoding genes, wherein said different genomic locus comprises transcriptional activity; and
c. regenerating the modified plant cell into a plant part or plant.

2. The method of claim 1 wherein said more than one mepsps genomic sequence or coding sequence are inserted into different genomic loci.

3. The method of claim 1, wherein said transcriptional activity is selected from constitutive expression, tissue-preferred expression, inducible expression, or increased expression compared to expression of said endogenous EPSPS gene.

4. The method of claim 1 or claim 2, wherein said inserting is accomplished by an approach selected from the group consisting of 5′ insertion, complete replacement, 3′ insertion, internal exon insertion, internal exon sequence replacement, internal intron insertion, and internal intron sequence replacement.

5. The method of claim 1, wherein said different genomic locus is a locus comprising a ubiquitin gene.

6. The method of claim 5, wherein the different genomic locus is the GmUbi3 as shown in SEQ ID NO:16, or sequence with at least 90% identity to SEQ ID NO:16.

7. The method of claim 1, wherein the different genomic locus is the GmERF10 genomic sequence as shown in SEQ ID NO:17, or sequence with at least 90% identity to SEQ ID NO:17.

8. The method of claim 1, wherein said mepsps encodes a sequence that when aligned with SEQ ID NO: 5 or 6 comprises at least one modified residue between residues 80 and 200 and wherein said plant has increased glyphosate tolerance compared to a plant comprising said SEQ ID NO: 5 or 6.

9. The method of claim 8, wherein said mepsps comprises two modified residues, said modifications comprising an isoleucine at residue 102 and a serine at residue 106.

10. An herbicide tolerant plant obtainable from the method of claim 1.

11. An herbicide tolerant plant, plant part, or plant cell comprising an insertion of a modified endogenous 5-enolpyruvylshikimate-3-phosphate synthase (mepsps) genomic sequence or coding sequence in a different genomic locus than endogenous EPSPS.

12. The herbicide tolerant plant, plant part, or plant cell of claim 11, wherein the said herbicide tolerant plant, plant part, or plant cell comprises more than one insertion of said mepsps genomic sequence or coding sequence.

13. The herbicide tolerant plant, plant part, or plant cell of claim 11 or claim 12, wherein said insertion occurs at a locus comprising a ubiquitin gene.

14. The herbicide tolerant plant, plant part, or plant cell of claim 11 or claim 12, wherein the different genomic locus is the GmUbi3 as shown in SEQ ID NO:16, or sequence with at least 90% identity to SEQ ID NO:16.

15. The herbicide tolerant plant, plant part, or plant cell of claim 11 or claim 12, wherein the different genomic locus is the GmERF10 genomic sequence as shown in SEQ ID NO:17, or sequence with at least 90% identity to SEQ ID NO:17.

16. The herbicide tolerant plant, plant part, or plant cell of claim 11 or claim 12, comprising the sequence shown in SEQ ID NO:18, or any sequence with at least 90% identity to SEQ ID NO:18 encoding mepsps protein.

17. Seeds of an herbicide tolerant plant, plant part, or plant cell comprising an insertion of modified 5-enolpyruvylshikimate-3-phosphate synthase (mepsps) genomic sequence or coding sequence in a different genomic locus.

18. The seeds of 15, wherein the seeds are non-transgenic.

19. A method for altering the expression of an endogenous plant gene, the method comprising:
a. providing a plant cell comprising an endogenous plant gene,
b. inserting a copy of genomic sequence or coding sequence of said endogenous plant gene or a sequence having at least 90% identity thereto into a different genomic locus from the locus of said endogenous plant gene, wherein said different genomic locus comprises transcriptional activity, and
c. regenerating a modified plant.

20. The method of claim 19, wherein said transcriptional activity is selected from constitutive expression, tissue-preferred expression, inducible expression, increased expression compared to said endogenous gene or decreased expression compared to said endogenous gene.

21. The method of claim 19, wherein said inserting is accomplished by an approach selected from the group consisting of 5′ insertion, complete replacement, 3′ insertion, internal exon insertion, internal exon sequence replacement, internal intron insertion, and internal intron sequence replacement.

22. The method of any one of claim 19, wherein the method further comprises inactivating said endogenous plant gene at its original genomic locus.

23. The method of any one of claims 19 to 22, wherein the method further comprises regenerating the modified plant cell into a plant part or plant.

24. A modified plant obtainable by the method according to claim 23.

25. A method for generating an herbicide tolerant plant, the method comprising:
a. providing a plant cell comprising one or more endogenous5-enolpyruvylshikimate-3-phosphate synthase (EPSPS) genes,
b. modifying the genetic material within said plant cell by inserting an EPSPS genomic sequence or coding sequence into a different genomic locus than said one or more endogenous EPSPS genes, wherein said different genomic locus comprises transcriptional activity, and
regenerating a modified plant.

26. The method of claim 25, wherein said transcriptional activity is selected from constitutive expression, tissue-preferred expression, inducible expression, increased expression compared to said endogenous gene or decreased expression compared to said endogenous gene.

27. The method of claim 25, wherein said inserting is accomplished by an approach selected from the group consisting of 5′ insertion, complete replacement, 3′ insertion, internal exon insertion, internal exon sequence replacement, internal intron insertion, and internal intron sequence replacement.

28. The method of claim 25, wherein said different genomic locus is a locus comprising a ubiquitin gene.

29. The method of claim 28, wherein the different genomic locus is the GmUbi3 as shown in SEQ ID NO:16, or sequence with at least 90% identity to SEQ ID NO:16.

30. The method of claim 25, wherein the different genomic locus is the GmERF10 genomic sequence as shown in SEQ ID NO:17, or sequence with at least 90% identity to SEQ ID NO:17.

31. The method according to any one of claims 25 to 30, wherein more than one endogenous EPSPS coding sequences are inserted.

32. The method according to any one of claims 25 to 30, wherein more than two endogenous EPSPS coding sequences are inserted.

33. The method according to any one of claims 25 to 30, wherein more than three endogenous EPSPS coding sequences are inserted.

34. The method of any one of claims 25 to 33, wherein the method further comprises a step of inactivating the endogenous plant gene at its original genomic locus.

35. The method of any one of claims 25 to 34, wherein the method further comprises a step of regenerating the modified plant cell into a plant part or plant.

36. An herbicide tolerant plant obtainable by the method according to any one of claims 25 to 35.

37. An herbicide tolerant plant, plant part, or plant cell comprising an endogenous 5-enolpyruvylshikimate-3-phosphate synthase (EPSPS) gene and an insertion of an EPSPS genomic sequence or coding sequence in a different genomic locus from said endogenous gene.

38. The herbicide tolerant plant, plant part, or plant cell of claim 37, wherein the said herbicide tolerant plant, plant part, or plant cell comprises one or more additional insertions of endogenous EPSPS genomic sequence or coding sequence.

39. The herbicide tolerant plant, plant part, or plant cell of claim 37, wherein said insertion occurs at a locus comprising a ubiquitin gene.

40. The herbicide tolerant plant, plant part, or plant cell of claim 37, wherein the different genomic locus is the GmUbi3 as shown in SEQ ID NO:16, or sequence with at least 90% identity to SEQ ID NO:16.

41. The herbicide tolerant plant, plant part, or plant cell of claim 37, wherein the different genomic locus is the GmERF10 genomic sequence as shown in SEQ ID NO:17, or sequence with at least 90% identity to SEQ ID NO:17.

42. The herbicide tolerant plant, plant part, or plant cell of claim 37, comprising sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, and SEQ ID NO:15; or harboring a sequence with 90% identity to the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, and SEQ ID NO:15.

43. Seeds of an herbicide tolerant plant, plant part, or plant cell comprising an endogenous 5-enolpyruvylshikimate-3-phosphate synthase (EPSPS) gene and an insertion of an EPSPS genomic sequence or coding sequence in a different genomic locus from said endogenous gene.

44. The seeds of claim 43, wherein the seeds are non-transgenic.

45. A method for generating an herbicide tolerant plant, the method comprising:
a. providing a plant cell comprising one or more endogenous 5-enolpyruvylshikimate-3-phosphate synthase (EPSPS) genes,
b. modifying the genetic material within said plant cell by inserting a mepsps genomic sequence or coding sequence into a different genomic locus, wherein said different genomic locus comprises transcriptional activity, and
c. regenerating the modified plant cell into a plant part or plant.

46. The method of claim 45 wherein more than one mepsps genomic sequences or coding sequences are inserted into different genomic loci.

47. The method of claim 45 or claim 46, wherein said inserting is accomplished by an approach selected from the group consisting of 5′ insertion, complete replacement, 3′ insertion, internal exon insertion, internal exon sequence replacement, internal intron insertion, and internal intron sequence replacement.

48. The method of claim 45, wherein said different genomic locus is a locus comprising a ubiquitin gene.

49. The method of claim 48, wherein the different genomic locus is the GmUbi3 as shown in SEQ ID NO:16, or sequence with at least 90% identity to SEQ ID NO:16.

50. The method of claim 45, wherein the different genomic locus is the GmERF10 genomic sequence as shown in SEQ ID NO:17, or sequence with at least 90% identity to SEQ ID NO:17.

51. An herbicide tolerant plant obtainable from the method of claim 45.

52. An herbicide tolerant plant, plant part, or plant cell comprising and endogenous 5-enolpyruvylshikimate-3-phosphate synthase (EPSPS) gene and an insertion of mepsps genomic sequence or coding sequence in a different genomic locus from said endogenous gene.

53. The herbicide tolerant plant, plant part, or plant cell of claim 52, wherein the said herbicide tolerant plant, plant part, or plant cell comprises more than one insertion of mepsps genomic sequence or coding sequence.

54. The herbicide tolerant plant, plant part, or plant cell of claim 52 or claim 53, wherein said insertion occurs at a locus comprising a ubiquitin gene.

55. The herbicide tolerant plant, plant part, or plant cell of claim 54, wherein the different genomic locus is the GmUbi3 as shown in SEQ ID NO:16, or sequence with at least 90% identity to SEQ ID NO:16.

56. The herbicide tolerant plant, plant part, or plant cell of claim 52 or claim 53, wherein the different genomic locus is the GmERF10 genomic sequence as shown in SEQ ID NO:17, or sequence with at least 90% identity to SEQ ID NO:17.

57. The herbicide tolerant plant, plant part, or plant cell of claim 52 or claim 53, comprising sequence shown in SEQ ID NO:18, or any sequence with at least 90% identity to SEQ ID NO:18, wherein SEQ ID NO:18 comprises sequence encoding mepsps protein.

58. Seeds of an herbicide tolerant plant, plant part, or plant cell comprising an endogenous 5-enolpyruvylshikimate-3-phosphate synthase (EPSPS) gene and an insertion of mepsps genomic sequence or coding sequence in a different genomic locus from said endogenous locus.

59. The seeds of claim 58, wherein the seeds are non-transgenic.

60. A method of changing expression of a gene product in a plant, comprising,
a) identifying a desired change in expression of a gene product in a plant and determining the transcription activity needed of a first gene in a first location encoding said gene product;
b) identifying at least one second location in the genome of said plant having said transcription activity;
c) inserting a nucleic acid sequence of said gene into said at least one second location, wherein said nucleic acid nucleotide sequence does not comprise a promoter; and
d) producing a plant that has changed expression of said gene product as a result of expression of said nucleic acid nucleotide sequence.

61. The method of claim 60, wherein said gene comprises a non-transgenic endogenous gene.

62. The method of claim 60, wherein said at least one second location has transcription activity that is selected from constitutive expression, plant tissue preferred expression, expressing said product at a lower level than the wild-type gene; expressing said product at a higher level than the wild-type gene, expressing when exposed to an inducer, or a combination thereof.

63. The method of claim 60 wherein said nucleic acid sequence is inserted at said second location by 5′ insertion, complete replacement, 3′ insertion, internal exon insertion, internal exon sequence replacement, internal intron insertion, and internal intron sequence replacement

64. A method of changing expression of a gene product in a plant, comprising
a) identifying a desired change in expression of a gene product in a plant and determining the transcription activity needed of an first endogenous gene in a first location encoding said gene product, said transcription activity selected from constitutive expression, plant tissue preferred expression, expressing said product at a lower level than the wild-type gene; expressing said product at a higher level than the wild-type gene, expressing when exposed to an inducer, or a combination thereof;
b) identifying at least one second location in the genome of said plant having said transcription activity;
c) identifying in said second location a target site in a second gene of said second location wherein insertion of nucleotide sequences at said site will retain the desired transcriptional activity;
d) inserting a nucleic acid sequence of said first gene or a modified nucleic acid sequence of said first gene into said at least one second location, wherein said nucleic acid sequence does not comprise a promoter, said nucleic acid sequence inserted by 5′ insertion, complete replacement, 3′ insertion, internal exon insertion, internal exon sequence replacement, internal intron insertion, or internal intron sequence replacement; and
e) producing a plant that has changed expression of said gene product as a result of expression of said nucleic acid sequence.

65. The method of claim 64 wherein said gene is selected from a 5-enolpyruvylshikimate-3-phosphate synthase (EPSPS) encoding gene, or yellow fluorescent protein gene.

66. The method of claim 63 wherein said nucleic acid sequence is a modified EPSPS (mepsps).

67. The method of claim 66, wherein said mepsps encodes a sequence that when aligned with SEQ ID NO: 5 or 6 comprises at least one modified residue between residues 80 and 200 and wherein said plant has increase glyphosate tolerance compared to a plant comprising said SEQ ID NO: 5 or 6.

68. The method of claim 66, wherein said mepsps comprises isoleucine at residue 102 and a serine at residue 106.

69. A method of increasing expression of 5-enolpyruvylshikimate-3-phosphate synthase (EPSPS) in a plant, comprising,
a) providing a plant comprising a first gene encoding EPSPS at a first genomic location;
b) identifying a second location in the genome of said plant having transcription activity selected from increased transcription activity compared to said first gene expression, or constitutive expression, or both;
c) inserting a nucleic acid sequence of said EPSPS gene or a modified nucleic acid sequence of said EPSPS gene into said at least one second location, wherein said nucleic acid molecule does not comprise a promoter; and
d) producing a plant that has increased expression of EPSPS as a result of expression of said nucleic acid sequence.

70. The method of claim 69 wherein said second location comprises a ubiquitin promoter.

71. The method of claim 70 wherein said second location comprises GmUbi3 as shown in SEQ ID NO: 16, or a sequence with at least 90% identity to SEQ ID NO: 16.

72. The method of claim 69 wherein said second location comprises GmERF10 as shown in SEQ ID NO: 17 or a sequence with at least 90% identity to SEQ ID NO: 17.

73. The method of claim 69 wherein said EPSPS gene encodes a polypeptide selected from SEQ ID NO: 5 or 6 or a sequence having at least 90% identity thereto and which retains the function of providing herbicide tolerance to a plant.


SEQUENCE LISTING (31 sequences)

SEQ ID NO: 1 - DNA, 8218 bp, Glycine max
atggcccaagtgagcagagtgcacaatcttgctcaaagcactcaaatttttggccattcttccaactccaacaaactcaaatcggtgaattcggtttcattgaggccacgcctttggggggcctcaaaatctcgcatcccgatgcataaaaatggaagctttatgggaaattttaatgtggggaagggaaattccggcgtgtttaaggtttctgcatcggtcgccgccgcagagaagccgtcaacgtcgccggagatcgtgttggaacccatcaaagacttctcgggtaccatcacattgccagggtccaagtctctgtccaatcgaattttgcttcttgctgctctctctgaggtgaagtttatttatttatttatttgtttgtttgttgttgggtgtgggaataggagtttgatgtgtagagtggattttgaatatttgatttttttttgtattattctgtgaaaatgaagcatcatgtcccatgaaagaaatggacacgaaattaagtggcttatgatgtgaaatgaggatagaaatgtgtgtagggttttttaatgggtagcaataagcatattcaatatctggattgatttggacgtttctgtataaaggagtatgctagcaatgtgttaatgtatggcttgctaaaatactcctaaaaatcaagtgggagtagtatacatatctacagcaaatgtattaggtgaggcatttggcttctctattgtaaggaacaaataatatcagttaatgtgaaaatcaatggttgatattccaatacattcatgatgtgttatttatatgtacctaatattgactgttgtttttctccgcaatgaccaagattatttattttatcctctaaagtgactaattgagttgcttactttagagaagttggacccattaggtgagagcgtggggggaactaatcttgaatatacaatctgagtcttgattatccaagtatggttgtatgaacaatgttagctctagaagataaaccctcccccaaaacacaaattagaatgacatttcaagttccatgtatgtcactttcattctattatttttacaacttttagttacttaacagatgtcttgttcagcataaattataatttattctgtttttttttagggaacaactgttgtagacaacttgttgtatagtgaggatattcattacatgcttggtgcattaaggacccttggactgcgtgtggaagatgacaaaacaaccaaacaagcaattgttgaaggctgtgggggattgtttcccactagtaaggaatctaaagatgaaatcaatttattccttggaaatgctggtactgcaatgcgtcctttgacagcagctgtggttgctgcaggtggaaatgcaaggtctgttttttttttttttgttcagcataatctttgaattgttcctcgtataactaatcacaacagagtacgtgttcttcttcctgttataatctaaaaatctcatccagattagtcatcctttcttcttaaaaggaacctttaattatcaatgtatttatttaatatttaaattagcttgtcaaagtctagcatatacatattttgattatattctgagaaatgcacctgagggtgttcctcatgatctacttcaacctctgttattattagattttctatcatgattactggtttgagtctctaagtagaccatcttgatgttcaaaatatttcagctacgtacttgatggggtgccccgaatgagagagaggccaattggggatttggttgctggtcttaagcaacttggtgcagatgttgattgctttcttggcacaaactgtccacctgttcgtgtaaatgggaagggaggacttcctggcggaaaggtatggtttggatttcatttagaataaggtggagtaactttcctggatcaaaattctaatttaagaagcctccctgttttcctctctttagaataagactaagggtaggtttaggagttgggttttggagagaaatggaagggagagcaatttttttcttcttctaataaatattctttaatttgatacattttttaagtaaaagaatataaagatagattagcataacttaatgttttaatcttttatttatttttataaatattatatacctgtctatttaaaaatcaaatatttgtcctccattccctttcccttcaaaacctcagttccaaatataccgtagttgaattatattttggaaggcctattggttggagacttttccttttcagagattatccctcacctttattatagcctttctatttttaaacttcatatagacgccattcttgttttaaaaaacactaagttttcttttagttataccttcccctccttattctctccaaagtaaatattgtagcaagtgttggttgatccattgtgaatcttattatcctatttaacatgcaggtgaaactgtctggatcagttagcagtcaatacttgactgctttgcttatggcagctcctttagctcttggtgatgtggaaattgagattgttgataaactgatttctgttccatatgttgaaatgactctgaagttgatggagcgttttggagtttctgtggaacacagtggtaattgggataggttcttggtccatggaggtcaaaagtacaagtaggtttctatgttttagtgttacatcacttttagtatccaaaatgcaatgaaattaaaactcatgtttcctattaggtctcctggcaatgcttttgttgaaggtgatgcttcaagtgccagttatttactagctggtgcagcaattactggtgggactatcactgttaatggctgtggcacaagcagtttacaggtattcatgaatgacactatcttctattatcttttattttatagcttacactatcattcctaattcaacccccttcttttctctttcccctcactcccaaaatagagcttatcattgtatccttaatagctcttttatgtgctgtttcatctcttgagcagattctacatgattatcctttttagtgaacttttttgtcttttaaataattgatatcatcatttgaacttgcacatcatctctatccttcctcattttattatatttcattaagagtgataaaattacattgtcaagtaaagcaagttagcgtatcagataatgatgcttcgattgcagaattggtgaaagcttgcacaattcattagcttagtgacaattgtgatgtagatagccataatcttccatgcaacttattatgctttttgtctaaaacttcttttgcacttaaacatgaattagagagttaggatgaacaatagtaacagacacaaagcttatttctagaccacccagtggtgaagacgaagcagcatgagtgagattaatggcagcatctgattgaagcttcttttggtattattggagatgaaatcttgagccaaattagggcttttgcctcctcaatagccattggttcaaaattgctttcaataaaatgattacagaatcaaattcttaagaaagaccttcaagaacaacatcaagatgtttcctgtgcaaaacaggctcacctacaaaagagtgtctacaagcacctttacacttgcaaaaaattccttaactgaacaattgccaagagtcatgttgcaaacttgagattggagttgattagatttggtgtgtgtgagattctggaaatgagcatgaagttttttgcaaagctcataaaatgaatgcagccaagaatcctagataggattgaatttgagagagaggcttgaagtcaagataaccgcaactgatcttgctgttcccatgctgcataggactcgttgattctaccaagataacaatcttctgtggtgagaatatgagtgggaattgtcaggcaacaaagaaaatgaaggctatgagcctttgttaaggtgcagagagaaaacagaaaatgaagaacttgtgtattgaaggaattgaaaagccaattacaattgtgttgcagagagatatttatagttctgaagtgtataactaactaaacagaatgctaccaacctagaggcagttgagaattgctcccttattacaatactctcacagccttgatgataggcttgatttgctgccaccgaagaagaacattattgttggagagtttttcagtgattgcatcagagaaagttacaagtgtgattggagatgaaggacttgcggtcgtcgaaggttaagtgttaagtggtggaatgtgtatcggaggtggttgcctagcccaagaagctggttgcagtggaagcggatgcggcagaagccatgggttcagatcagaactatgttctagataccatattagggttagctttcaagagaaaacttatgaaactgaactgtaaactgttgtattattgattagctgatctagtagtgatggaagcttgcttgcgaagcttctatggaggctggatcattgagcttcaatgaggtccttcaatggtgatttttcaccatggagatgcagcggaagataaatgaagaggtgagaggaggcgtcatccactaggaaataaactatggaagaaggagcttcaccaccaagagagtgtcttggataagaagcttagagaggaagcttcaatggaggaatagaaagagagagggggaacaaaaaattgaaggaggaaaagagggagagaagttgaactttgaagtgtgtctcacaagactcattcattaaagttacaacaagtgttacacatgcttctatttatagtctaggtaacttccttgagaagctagaacttaactacacacacctctctaataactaagataacctccttgagaaatttccttgagaagcttccttaagaagattcctagagaagctaaagcttagttacacacacctctctaataactaagttcacctccttgagatgagaagctagagccttagctacacacacccttataatagctaaactcactctattccaaaatacatgaaaatacaaaaaagttcctactacaaagattactcaaaatgtcctgaaatacaagactaaaactctatactactagaatggtcaaaatacaaggtccaaaagaaggaaaaacctattctaatatttacaaagagagtggacccaaccttagtccatgggctcagaaatctaccctgaggttcatgggaatcctagggtcttctttagtagctctagtccaattttcttggagtcttctatccaatatccttgggggtaggattgcatcatgtagtacaagagaggggaatatatatacagttgtaagttgtaactacctaatcaaacccaactgactcaccatacacagaaaaacaatgtacaaaggataatagaaaaactgaaactgagatatactcattacatagaagaattacatgatgaatctgaaaattttaattgggaaactgaggacaataacaaggctttcagagttcattttgacaataggtaaattatggacttcaacttagacctcaagggtatgcttgggagtggggttttagaggaaaaggggaaggagggaaattttttaattctagtaaatattctctattttgattaatttttaaaataaaacaaataaacttatagattagcattcacttaatgtcctgatcttttatttgtttcatgaagttttttatagcctttctgtttctgttaaaagaaattggagggtgttcataatctatatgcccatatatatgtattgtcctgttgtctgtgtttctgttttctttctttagtgtctcgcctttccttaattccttatcaacaccttgtttatttttgaaaacttaaatgtcctcattttttttacctgtactataactttccaaacaaatagtagaaaatgtttcaaatattccccagaactgtttcagtgaacaaagttcaagccagaaaaaaagcaagcagaggctaaagaagtatatcttattgtgaagagtaatctcaattggttaattatcgaattgtatcattttacaatagaaaattagaagtgggttgattagtttatacgaacaattatttaaatagggttgatggtgtggttaattactgatcatatgttaagactatgagtcgatgaccagcctcagtgcagtcattttaagttccattattttttaattgtgcttatgtttaactttgtccttcttttaaagggagatgtaaaatttgctgaagttcttgaaaagatgggagctaaggttacatggtcagagaacagtgtcactgtttctggaccaccacgagatttttctggtcgaaaagtcttgcgaggcattgatgtcaatatgaacaagatgccagatgttgccatgacacttgctgttgttgcactatttgctaatggtcccactgctataagagatggtatggtttagctgtttgtttttgctcaagagcttgttttcacatatttgtggtcaagagcggtttagctgtttttttttttaaattgtggttaatgaaattattataattatactaatattgaaattggatcactaaccatgtgaaatgcttgaatttttagcttgaaaacttcttttgtggatgatttttcttttatctatgtagtgtggggcatctatgttgttcctttaagctttgtgtcatagttgtacaacctggtctgtgggctggtccagttcttactcatgactgtcttttgcagcaaactggttaaacttgggtgacctgttgacccacccagattggcctgttcttttttaaataaaactccaaaaggctacttataagatttcaacatgatacttgaatgaccacccttactttaaaggtcattatgccatcagaccccttgattctctgttcagttatgttcttattcttattattttttaatttatattattatccaacgatctgatctttgtacatgatgctggacaatcatcaaagctagtttggttacatcatataataccttttttttcataaattattttttcccataatatatttatatgtatttttttaatacaatattctggggaaattttttttttctgttttcttttttctggtttgaatactatttcaatgactgggccttattattttctggtttgggttatacaactgtcctttgatgtacttttgaatgcttcaatagtatttttcataatcatggaaggcactagagtgcagtgcatattgcacaaggttctttaatggcatcttgaatttgttagaacatggagttatgatatacaaccattcctatgtcttcatctaatagctaaagcttttggcgtagttggttaacatggtattggaatcttattttaaaggtccatcttattcgcaagtacaaggcaggtggacctgcgcattattcacgtgccaggctcaaagggattttgtatgagggggaatttttggaatataatatatagaatcattcatgtggttcacctatcatgtgtcaactcaagtttttgtcctagttgtattattgcagtgtctaaactaggcatgatcttctgtggaaggaaatttaacctaaggaacttgaatgcttcttgttctgagatctttattttttttctccaatctttttaatacttgaaaggttttagttgtatctataaagggtgcagaattagggggtcagcttttctgttgtaagcgtgaattaagtgcattttatgttgttccctattttatacatttgaactatcaaccatatcatttagcaaacttctttgttgctcagattcccatttattcttgtttcctctattttttagtggcaagttggagagttaaagagactgagaggatgatagcaatctgcacagaactcagaaaggtcttgctaattcctttatggtttctatattactggactttttacatctcactgacctactactgtcttggtatttcagctaggagcaacagttgaagaaggtcctgattactgtgtgattactccacctgagaaattgaatgtcacagctatagacacatatgatgaccacagaatggccatggcattctctcttgctgcttgtggggatgttccagtaaccatcaaggatcctggttgcaccaggaagacatttcctgactactttgaagtccttgagaggttaacaaagcactaa

SEQ ID NO: 2 - DNA, 1578 bp, Glycine max
atggcccaagtgagcagagtgcacaatcttgctcaaagcactcaaatttttggccattcttccaactccaacaaactcaaatcggtgaattcggtttcattgaggccacgcctttggggggcctcaaaatctcgcatcccgatgcataaaaatggaagctttatgggaaattttaatgtggggaagggaaattccggcgtgtttaaggtttctgcatcggtcgccgccgcagagaagccgtcaacgtcgccggagatcgtgttggaacccatcaaagacttctcgggtaccatcacattgccagggtccaagtctctgtccaatcgaattttgcttcttgctgctctctctgagggaacaactgttgtagacaacttgttgtatagtgaggatattcattacatgcttggtgcattaaggacccttggactgcgtgtggaagatgacaaaacaaccaaacaagcaattgttgaaggctgtgggggattgtttcccactagtaaggaatctaaagatgaaatcaatttattccttggaaatgctggtactgcaatgcgtcctttgacagcagctgtggttgctgcaggtggaaatgcaagctacgtacttgatggggtgccccgaatgagagagaggccaattggggatttggttgctggtcttaagcaacttggtgcagatgttgattgctttcttggcacaaactgtccacctgttcgtgtaaatgggaagggaggacttcctggcggaaaggtgaaactgtctggatcagttagcagtcaatacttgactgctttgcttatggcagctcctttagctcttggtgatgtggaaattgagattgttgataaactgatttctgttccatatgttgaaatgactctgaagttgatggagcgttttggagtttctgtggaacacagtggtaattgggataggttcttggtccatggaggtcaaaagtacaagtctcctggcaatgcttttgttgaaggtgatgcttcaagtgccagttatttactagctggtgcagcaattactggtgggactatcactgttaatggctgtggcacaagcagtttacagggagatgtaaaatttgctgaagttcttgaaaagatgggagctaaggttacatggtcagagaacagtgtcactgtttctggaccaccacgagatttttctggtcgaaaagtcttgcgaggcattgatgtcaatatgaacaagatgccagatgttgccatgacacttgctgttgttgcactatttgctaatggtcccactgctataagagatgtggcaagttggagagttaaagagactgagaggatgatagcaatctgcacagaactcagaaagctaggagcaacagttgaagaaggtcctgattactgtgtgattactccacctgagaaattgaatgtcacagctatagacacatatgatgaccacagaatggccatggcattctctcttgctgcttgtggggatgttccagtaaccatcaaggatcctggttgcaccaggaagacatttcctgactactttgaagtccttgagaggttaacaaagcactaa

SEQ ID NO: 3 - DNA, 7534 bp, Glycine max
atggcccaagtgagcagagtgcacaatcttgctcaaagcactcaaattttcggtcattcttccaatcccaacgaacccaaatcggcgaattcggtttcattgaggccacgcctttggggtccctcgaaatctcgcatcttggtgcacaaaactggaagccttatgggaaattttaatgcggggaagggaaattccggcatgtttaaggtttctgcctccgtcgccgccgccgcagagaagccttcgacggcgccggagatcgtgttggaacctatcaaagacatctcgggtaccatcacattgccagggtctaagtctctgtccaatcgaattttgcttcttgctgctctctctgaggtaaagtttatttatttattttttctgtatccaaaaatgtaaattgttagtttgttaatttttgttagcagtgagagtcgaactacttggcttctttcttagtcatccaaccaaccttatatctccaaaatttatttattggttttttttggggttatgtggttaggaataggagtttgatgcgtggagtggattttgaatatttgattttttttttgtattattcagtgaaaatgaagcatcttgtcccatgaaagaaatggacacgaaattaagtggcgtatgatttagaatgatgatagaaatgtgtataggtggttttaatgtgtagcaataagcatattcaatatctggattgatttggacgtttctgtataaaggagtatgctagcaatgtgttaataatgtcttgttaaaagtatggatggctaaaataatcataaaaatcgagtgggagtagtatacatatctacaggaaatgtattaggtgaggcatttggcttctctattgcgagtaaggaacaagtaatctcagttaatgtgaaaatcaatggttgatattccaatacattcatgatgtgttatttacgggaacgcaatattgactgttgattttatctgcagctgagattatatactatatccttccaaagaaatctttgactcttgattatccaagtatggttgtattaccaattttagctctagaagataatccctcccccaaaacacaaattagaatggtgctgcaagttctgtgttactttcattctattatttttataacttttaattatttaatagatgtcttgtttggcataaactataatttattctgtttttttttatttacttatttagggaacaactgttgtagacaacttgctgtacagcgaggatattcattacatgcttggtgcattaaggacccttggactgcgtgtggaagacgaccaaacaaccaaacaagcaattgtggaaggctgtgggggattgtttcccactattaaagaatctaaagatgaaatcaatttattccttggaaatgctggtactgcgatgcgtcctttgacagcagctgtagttgctgcaggtggaaatgcaaggtctgttttttgttttgtttgttcatcatgatctctgaattgttcctcgtataactaatcacatcagactatgtgttcttcctccatcctgttataatctaaaaatctaatccagattagtcatccttctttaaatgaacctttaattatatctatgtatttatttaacatgtaaattagcttgtcaagtcaaagtctagcatatagatatactgattacactctgaggaatgcacctgagggtcttactcatgatctacttcaaccttgccactttcttcttttattattagatcacctatcatgattactggtttgagtctctaaatagaccatcttgatgttcaaaatatttcagctacgtacttgatggagtgccccgaatgagagagaggccaattggggatttggttgctggtcttaagcagctcggtgcagatgttgattgctttcttggcacaaactgtccacctgttcgtgtaaatgggaagggaggacttcctggcggaaaggtatggtttggatttccattagaataaggtggactaactttcctggagcaaaattctaatttaaaccctgtttccctctctttagaataagacactaagggtatgtttaggagttgggttttggcgagaaagggaagggagggcaattttttttctaataaatattctttaatttgataaaatttttaaacgaaggaatatgaagatagattagcataacttaatgttttaatcttttatttatttttataaatattatatacctctatttaaaaacaagatatttttcctccattccctttccctttgaaacctcagttccaaatataccgtacttgaattatattttggaaggtgtattggttggagacctttccttttcagaggttatccctcacctttattatagcctttctactctctcaatgagttcattgtgcattgagtcattgaacttcctgtgaaaagaaattgtttacttttctttatttgttacctacatccttattcctgttttaaaaaatactaagttttcttttagttatgccttcccctccttattctctccaaagaaaatataatagcgaatgttggtttatccattgtgaatcttattatcctatttcacatgcaggtgaaactgtctggatcaattagcagtcaatacctaactgctttgcttatggcagctcctttagctcttggcgacgtggaaattgagattgttgataaactgatttctgttccatatgttgaaatgactctgaagttgatggagcgttttggagtttctgtggaacacagtggtaattgggataagttcttggtccatggaggtcaaaagtacaagtaggtttctatgttttagcattacatcactttttagtatccaaaatgcaatgaaatcaaaactcatgttttctatcaggtctcctggcaatgcttttgttgaaggtgatgcttcaagtgccagttacttcctagctggtgcagcagttactggtgggactatcactgttaatggctgtggcacaaacagtttacaggtattcatgcttgacactattttctattatcttttattttatggtctatactgtatcattcctaattcaacccccttcttttctctttcccctcacttccaaaaaaatagagcttatcattgtattcttaatagctcttgtatgtcctttttcgtctcttgagcagattctaccgtgattatcctttttagtaaacttttttttgtgtgtcttttaaataattgatatcatcatgtgaactcacacatcatctctatcccttcctcattttattatatttcattaacagtgataaagataaaaataaattgtcaagtaaagcaagttggcatgtcagataatgatgcttcaattgcaaaattggtgaaagcttgcacagtacattagctttggtgacaattgtgatgtagatagccataacctcccatgcaacttattatgttgtttgcttaaacttcttttgcacttaaacacggattagagagagtgaggatgaacaataataacagacacaaagcttagttttataccacccggcacccagtggtgaggaccaagcagcatgagtgagatcaatggcagcatctgattgaagcttcttgtggtatttttggagatgaaatcttgaggcgaattagggcatctggcttaacaatagccattggttcaaatttgcttttaataaaattgattacagaatcaaattcttgaggaagccttcaagaactacatcatgatgttccctgtgcaaaacaggttcgccgacaaaagagaacatctacaagttcctttacacatgccaaaaattcctcaactgaacaattgccaagagtcatgttgcgaatttcaggttggagttgacgaaatttggtgcgtgtatgattctggaaatgagcatgaagttttttcctaagctcataaaagtgaatgcagccaagaaccctagataggattgaatcggagagaggcttgaagccaagataacagcaactgatcttgctgttcccatgctgcgtaggactcattgattctaccaagatcacaatcttctgtggtgagaaaatggggtgaattgtcaaccaatagagaaaatggtgaaggctgagtcttgatgataggcttgatttgcttgcgccgaagaagaacgttattgttgtagagtttctcagtgattgtatgagagtaagttacaggtgcgattggagatgacggacttgtggtgggaggagggtaagtgttaagtggtggaatgtgtatcagaggtggttgcccagccaaagaagctggttgcggtggtgcagtggaagccatgggttttgatgcttttgagagaaaacatatgaaactgaactgtaaacttttgtattattgattagctgatttggtagtacaagagaggaatatatatacagttttaatgtaactacctaaccaaacatgaggaactctccatacacagaaagacgtatacaaaggataacagacaaacagaaacttagatatactcatacagagaagaattacaggatgaatctgagaattttaattgggaaactgaggaaaatatcaagggtttcagaattcattttgacgataggcaaatcatggacttaacttagacctcaagggtacgcttgggagtgggggtttttagagggaaaagggaaggagggcaaacttttaattcctaataaatattctcttttttgatttttttttaaaataaaacaaataaacttatagattagcatacacttaatgtcctaatcttttatttatttgcatgagtttttttcttttatattctttctgtttctgttttctttctttagtgtctcttgtctttccttaattccttatcaataccttgtttatttttgaaaacttaattttcctcatttttttaacctataggctatactatagtatgtataactattcaaataaatagtagaaaatgtttcaaatattccccagaactgtttcagtgaacaaagttcaagccagaaaaaagaagtatatcttattgtgaagagtagtctcaagtggttaattgtcaaattgtatcattttagaatacaaaattagaagtggtttgattagtttatacgaacgattatttaaatagggttgatgatgtggttaattactgatcatatgtgaagactgtgagtcgatgaccagcccagtgcagtcattttaaattccattattttttaatttgtgcttatgttaaactttgtccttctttcaaagggagatgtaaaatttgctgaagttcttgaaaagatgggagctaaggttacatggtcagagaacagtgtcaccgttactggaccaccacaagattcttctggtcaaaaagtcttgcaaggcattgatgtcaatatgaacaagatgccagatgttgccatgactcttgccgttgtcgcactatttgctaatggtcaaactgccatcagagatggtatggtttagctgtttgtttgtgttcaagagcttgttttaacaaattgttttttttttaattatggttaatgaaattattataattacactaatattgaaattggatcactaaccatgtgaaatgcttgaatttttagcttgaaaacttcttttgtggacaatttttcttttatctatggattgtggggcatctatgttgttcctttaagcttcgtacaacctggtctgtgggctggtccagtccttcttactcatgacagtcttttgcagtaaactggttaaacttgggtgacctgttgacccacctagattggcctgttctttttaaataaaacttcaaaaggctacttataagatttcaacatgatacttgaatgatcacccttactctaaaggccattatctgttcagttatgttcttcttcttcttcttattatttttaatttatattattatccaacgatctgatctttgaacatgatgctggacaatcatcaaagctagtttggttacatcatataataccttttttttcatatattatttttcccataatatatttttgtgtttttttttcatacaatattattctgaaaaaaacgtgtttcgtttttttcctggcttgaatgctatttcaatgactgggccttattattttctggtttaggttatacaactgtcctccaattgacttttgaaggcttcaatagttttcttcttaatcatggaaggcactagagtgcatattgaatatgttagaacatggaaggcttaaatatctttttagtctttgcaatttaacgtttttttgcttttagtccttgcaaaattataatttttttttagtcctggctaattatgtttgttttgttttttgtccttatatgctttagataacactttttttgttactgttcaaagcactatctaaagtgctttaaggactaaacacaaaacaaacataatttgcaaggactgaaaaacaaaaaaataattttgcaaggacgaaaaaataaaaataaaaaacacaaaattgcaagaccaaaaatatatttaaaccaacatgaaattatggtataaaaccattcatacgtcttcatctaatagcttaagcttttgggatagttggttaacaatatggtattagagtcttatgatttttaggtggtctttgagttcaatccttgctgctcccagttctagttaatttctttttggtccacttcaaataaaaagttgaatttaagggtaaggcagctggacctgcacattaatcatgtgtcaggctcaaagggcttttgtatggggggcatgtttggaatataatatagaatcattcatgtggtttcacctatcaaggatcaactctagtttttgtcatacttgtattattacagtgtctaaactagacatgatctttttccggaaggaaatttaacttaaaaggaacttagatgcttcttgttctgtgatctttatttgtttattttttccaatctttttaacaattgaaaggttttagttgtatccaatctttttatacatttggactatctaccatatcatttagcaaacttgtttgctgctcagattctcctttggactatctaccatatcatttagcaaacctgttcactgctcagattctcatttattctttgtttcctctatttttcagtggcaagttggagagttaaagagactgagaggatgatagcaatctgcacagaactcagaaaggtgagtgtcttgctaattcctttatggtttcgatattactgaactttttacatctcactgacctactctgtcttggtatttcagctaggagcaacagttgaagaaggtcctgattactgtgtgattactccacctgagaaattgaatgtcacagctatagacacatatgatgaccacagaatggccatggcattctctcttgctgcttgtggggatgttccagtaaccatcaaggatcctggttgcaccaggaagacatttcccgactactttgaagtccttgagaggttcacaaggcactaa

SEQ ID NO: 4 - DNA, 1581 bp, Glycine max
atggcccaagtgagcagagtgcacaatcttgctcaaagcactcaaattttcggtcattcttccaatcccaacgaacccaaatcggcgaattcggtttcattgaggccacgcctttggggtccctcgaaatctcgcatcttggtgcacaaaactggaagccttatgggaaattttaatgcggggaagggaaattccggcatgtttaaggtttctgcctccgtcgccgccgccgcagagaagccttcgacggcgccggagatcgtgttggaacctatcaaagacatctcgggtaccatcacattgccagggtctaagtctctgtccaatcgaattttgcttcttgctgctctctctgagggaacaactgttgtagacaacttgctgtacagcgaggatattcattacatgcttggtgcattaaggacccttggactgcgtgtggaagacgaccaaacaaccaaacaagcaattgtggaaggctgtgggggattgtttcccactattaaagaatctaaagatgaaatcaatttattccttggaaatgctggtactgcgatgcgtcctttgacagcagctgtagttgctgcaggtggaaatgcaagctacgtacttgatggagtgccccgaatgagagagaggccaattggggatttggttgctggtcttaagcagctcggtgcagatgttgattgctttcttggcacaaactgtccacctgttcgtgtaaatgggaagggaggacttcctggcggaaaggtgaaactgtctggatcaattagcagtcaatacctaactgctttgcttatggcagctcctttagctcttggcgacgtggaaattgagattgttgataaactgatttctgttccatatgttgaaatgactctgaagttgatggagcgttttggagtttctgtggaacacagtggtaattgggataagttcttggtccatggaggtcaaaagtacaagtctcctggcaatgcttttgttgaaggtgatgcttcaagtgccagttacttcctagctggtgcagcagttactggtgggactatcactgttaatggctgtggcacaaacagtttacagggagatgtaaaatttgctgaagttcttgaaaagatgggagctaaggttacatggtcagagaacagtgtcaccgttactggaccaccacaagattcttctggtcaaaaagtcttgcaaggcattgatgtcaatatgaacaagatgccagatgttgccatgactcttgccgttgtcgcactatttgctaatggtcaaactgccatcagagatgtggcaagttggagagttaaagagactgagaggatgatagcaatctgcacagaactcagaaagctaggagcaacagttgaagaaggtcctgattactgtgtgattactccacctgagaaattgaatgtcacagctatagacacatatgatgaccacagaatggccatggcattctctcttgctgcttgtggggatgttccagtaaccatcaaggatcctggttgcaccaggaagacatttcccgactactttgaagtccttgagaggttcacaaggcactaa

SEQ ID NO: 5 - protein, 525 aa, Glycine max
MAQVSRVHNLAQSTQIFGHSSNSNKLKSVNSVSLRPRLWGASKSRIPMHKNGSFMGNFNVGKGNSGVFKVSASVAAAEKPSTSPEIVLEPIKDFSGTITLPGSKSLSNRILLLAALSEGTTVVDNLLYSEDIHYMLGALRTLGLRVEDDKTTKQAIVEGCGGLFPTSKESKDEINLFLGNAGTAMRPLTAAVVAAGGNASYVLDGVPRMRERPIGDLVAGLKQLGADVDCFLGTNCPPVRVNGKGGLPGGKVKLSGSVSSQYLTALLMAAPLALGDVEIEIVDKLISVPYVEMTLKLMERFGVSVEHSGNWDRFLVHGGQKYKSPGNAFVEGDASSASYLLAGAAITGGTITVNGCGTSSLQGDVKFAEVLEKMGAKVTWSENSVTVSGPPRDFSGRKVLRGIDVNMNKMPDVAMTLAVVALFANGPTAIRDVASWRVKETERMIAICTELRKLGATVEEGPDYCVITPPEKLNVTAIDTYDDHRMAMAFSLAACGDVPVTIKDPGCTRKTFPDYFEVLERLTKH

SEQ ID NO: 6 - protein, 526 aa, Glycine max
MAQVSRVHNLAQSTQIFGHSSNPNEPKSANSVSLRPRLWGPSKSRILVHKTGSLMGNFNAGKGNSGMFKVSASVAAAAEKPSTAPEIVLEPIKDISGTITLPGSKSLSNRILLLAALSEGTTVVDNLLYSEDIHYMLGALRTLGLRVEDDQTTKQAIVEGCGGLFPTIKESKDEINLFLGNAGTAMRPLTAAVVAAGGNASYVLDGVPRMRERPIGDLVAGLKQLGADVDCFLGTNCPPVRVNGKGGLPGGKVKLSGSISSQYLTALLMAAPLALGDVEIEIVDKLISVPYVEMTLKLMERFGVSVEHSGNWDKFLVHGGQKYKSPGNAFVEGDASSASYFLAGAAVTGGTITVNGCGTNSLQGDVKFAEVLEKMGAKVTWSENSVTVTGPPQDSSGQKVLQGIDVNMNKMPDVAMTLAVVALFANGQTAIRDVASWRVKETERMIAICTELRKLGATVEEGPDYCVITPPEKLNVTAIDTYDDHRMAMAFSLAACGDVPVTIKDPGCTRKTFPDYFEVLERFTRH

SEQ ID NO: 7 - DNA, 5378 bp, Artificial Sequence
  pUbi3Ubi3T2AEPSPS chr 1 CDS
gtgaaaattttcacaagattttgaaagctctgaactcattagcacctgatttaatggttagaccaggctcaactgtgcctgcaaaaataagggaaagcacaaggttttatccttattttaaggtatgtgatcattatctaattataacaaaattataattataattgtgatggcatgtgcagcacttcataattttcttcgcaaagaatgtcgttctgatgaatttccagtggaacctactgacgagtcttcatcttcatcttcagtgttaccaaattacgaagacaatgatcatgaacccattgttcaaacacaagagcaggaacgagaagatgctaatatatggaggactaatataggttcagatatgtggagaaatgctaataattaggcgaacatgaagtgagaatcactttgttattatttttttaggcaataatgactttgttattaaaaggtttaaaatttctatcgtttttattttctttattcaaacattataatttatttatcatctttttcattcacttttgtaacttccgttatttttttgtttaaaatgtattaatctttcaaaatcttaaaaatccataaagtactttgaaatcttaaaaatctgtgttagaaatccattaaaatcttgggttaagaatcctgattgtaaaaagtcttttaaaaaaaatcttttaaaatcccacaaaatcaatacaatcccacataatcttttaaaatcttcaagattgtttttgtcaaaatattctctcaaaatcccaatccaatacaccccccttaattaacaaaaatgaatacgggctaacatggttatgggcttaggctgcctcattacccattacatgctctgttcctaaaaaaaagaagtttgggcctttattctccagtaagctttttaattgggctcaagaactttaggtatttaggtgagattcaatgaaaatttatacattaggcttgatttttttttttcaatttacgcaaaaatgaaagaaaaagtcacactctataaactttatttttttcaatattaatacattgttattgtggatatatcattagcggaaaagtaattctaataccagatttaattgactttgactaatttttttatgtgatcaaataatttttttagcgaaaaacttatatatatatatatatatatatatatatataagttttgttacttttccgcgtgatttttaatcaaactttggggataatttcccctccaattcagccaaaaaaaaaaaaaaactgacaccatatattattagtaggcaacttgttcgtaaatggtgtggcttatgcgaatggaaattggattcgttttctttaacatcattattgtttttgtcaatgagctatcttttagtcttatgttattggtgaatctgtccttaagttgcagcatttaacacatctcctcattagagaaaaaaattcttccctaaacgatagtaaaaacatctaataagaaataagaaagaaaaattaggaaaaagaaaagttcattaaaaaaatcttttggattatttttaaaaaaatatctaaatattttttaaatgaataattttatataaactgtaactaaaagtatacaagtaatgtatgttaacaaaatacttgaaaaatctactgaaaatatatcttacaaagtgaaattaaataagaaagaatttagtggaataattatgattttatttaaaaaataattattaaagatttttttgctccataataagaaaacttttcaattattcttttctggtccataataaaaaaaatctagcatgacagcttttccatagatttttaataatgtaaaagcagccgacttcaggcaatggatagtggggcccgtatcaacttcggacgctccacttgcaacggggtgggcccaatataacaacgacgtcgtaacagataaagcgaagcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacagatgcagatcttcgtcaagaccctcaccggcaagaccatcacccttgaggtggaaagctctgacaccatcgacaacgtcaaggccaagatccaggacaaggaaggaatccccccggaccagcaacgtctcattttcgccggaaagcaacttgaggacggccgtacccttgctgactacaacattcagaaggagagtactcttcacctcgtcctccgtctccgtggtggcatgcagatcttcgttaagacactcaccggcaagaccataaccctagaggttgaaagctccgacaccatcgataacgtcaaggccaagatccaggacaaggagggtatccccccggaccagcaacgtctcatcttcgccggaaagcagctcgaggacggccgcaccctcgccgactacaacatccagaaggaatcaacccttcacctcgtcctccgtctccgtggtggcatgcagatcttcgttaagaccctcaccggcaagactattaccctagaagtcgaaagctccgacaccatcgacaacgtcaaggctaagattcaggacaaggagggaatccccccagaccagcagaggctgatcttcgccgggaagcagctcgaggacggacgcacccttgctgactacaacatccagaaggagtcaactctccacttggtgttgcgtcttcgtggtggtatgcagattttcgtgaagactcttacgggtaagactattaccctcgaggtggagagctctgacaccattgacaatgtgaaggccaagattcaggacaaggaaggcatcccaccggaccagcagaggctgatttttgctggcaagcagctcgaggatggaaggaccctcgctgactacaacatccagaaggaatcaacccttcaccttgtcctccgtctccgtggggggtttgagggcagaggaagtcttctaacatgcggtgacgtggaggagaatcccggcccttctagaatggcccaagtgagcagagtgcacaatcttgctcaaagcactcaaatttttggccattcttccaactccaacaaactcaaatcggtgaattcggtttcattgaggccacgcctttggggggcctcaaaatctcgcatcccgatgcataaaaatggaagctttatgggaaattttaatgtggggaagggaaattccggcgtgtttaaggtttctgcatcggtcgccgccgcagagaagccgtcaacgtcgccggagatcgtgttggaacccatcaaagacttctcgggtaccatcacattgccagggtccaagtctctgtccaatcgaattttgcttcttgctgctctctctgagggaacaactgttgtagacaacttgttgtatagtgaggatattcattacatgcttggtgcattaaggacccttggactgcgtgtggaagatgacaaaacaaccaaacaagcaattgttgaaggctgtgggggattgtttcccactagtaaggaatctaaagatgaaatcaatttattccttggaaatgctggtactgcaatgcgtcctttgacagcagctgtggttgctgcaggtggaaatgcaagctacgtacttgatggggtgccccgaatgagagagaggccaattggggatttggttgctggtcttaagcaacttggtgcagatgttgattgctttcttggcacaaactgtccacctgttcgtgtaaatgggaagggaggacttcctggcggaaaggtgaaactgtctggatcagttagcagtcaatacttgactgctttgcttatggcagctcctttagctcttggtgatgtggaaattgagattgttgataaactgatttctgttccatatgttgaaatgactctgaagttgatggagcgttttggagtttctgtggaacacagtggtaattgggataggttcttggtccatggaggtcaaaagtacaagtctcctggcaatgcttttgttgaaggtgatgcttcaagtgccagttatttactagctggtgcagcaattactggtgggactatcactgttaatggctgtggcacaagcagtttacagggagatgtaaaatttgctgaagttcttgaaaagatgggagctaaggttacatggtcagagaacagtgtcactgtttctggaccaccacgagatttttctggtcgaaaagtcttgcgaggcattgatgtcaatatgaacaagatgccagatgttgccatgacacttgctgttgttgcactatttgctaatggtcccactgctataagagatgtggcaagttggagagttaaagagactgagaggatgatagcaatctgcacagaactcagaaagctaggagcaacagttgaagaaggtcctgattactgtgtgattactccacctgagaaattgaatgtcacagctatagacacatatgatgaccacagaatggccatggcattctctcttgctgcttgtggggatgttccagtaaccatcaaggatcctggttgcaccaggaagacatttcctgactactttgaagtccttgagaggttaacaaagcactaa

SEQ ID NO: 8 - DNA, 12018 bp, Artificial Sequence
  pUbi3Ubi3T2AEPSPS chr 1 genomic sequence
gtgaaaattttcacaagattttgaaagctctgaactcattagcacctgatttaatggttagaccaggctcaactgtgcctgcaaaaataagggaaagcacaaggttttatccttattttaaggtatgtgatcattatctaattataacaaaattataattataattgtgatggcatgtgcagcacttcataattttcttcgcaaagaatgtcgttctgatgaatttccagtggaacctactgacgagtcttcatcttcatcttcagtgttaccaaattacgaagacaatgatcatgaacccattgttcaaacacaagagcaggaacgagaagatgctaatatatggaggactaatataggttcagatatgtggagaaatgctaataattaggcgaacatgaagtgagaatcactttgttattatttttttaggcaataatgactttgttattaaaaggtttaaaatttctatcgtttttattttctttattcaaacattataatttatttatcatctttttcattcacttttgtaacttccgttatttttttgtttaaaatgtattaatctttcaaaatcttaaaaatccataaagtactttgaaatcttaaaaatctgtgttagaaatccattaaaatcttgggttaagaatcctgattgtaaaaagtcttttaaaaaaaatcttttaaaatcccacaaaatcaatacaatcccacataatcttttaaaatcttcaagattgtttttgtcaaaatattctctcaaaatcccaatccaatacaccccccttaattaacaaaaatgaatacgggctaacatggttatgggcttaggctgcctcattacccattacatgctctgttcctaaaaaaaagaagtttgggcctttattctccagtaagctttttaattgggctcaagaactttaggtatttaggtgagattcaatgaaaatttatacattaggcttgatttttttttttcaatttacgcaaaaatgaaagaaaaagtcacactctataaactttatttttttcaatattaatacattgttattgtggatatatcattagcggaaaagtaattctaataccagatttaattgactttgactaatttttttatgtgatcaaataatttttttagcgaaaaacttatatatatatatatatatatatatatatataagttttgttacttttccgcgtgatttttaatcaaactttggggataatttcccctccaattcagccaaaaaaaaaaaaaaactgacaccatatattattagtaggcaacttgttcgtaaatggtgtggcttatgcgaatggaaattggattcgttttctttaacatcattattgtttttgtcaatgagctatcttttagtcttatgttattggtgaatctgtccttaagttgcagcatttaacacatctcctcattagagaaaaaaattcttccctaaacgatagtaaaaacatctaataagaaataagaaagaaaaattaggaaaaagaaaagttcattaaaaaaatcttttggattatttttaaaaaaatatctaaatattttttaaatgaataattttatataaactgtaactaaaagtatacaagtaatgtatgttaacaaaatacttgaaaaatctactgaaaatatatcttacaaagtgaaattaaataagaaagaatttagtggaataattatgattttatttaaaaaataattattaaagatttttttgctccataataagaaaacttttcaattattcttttctggtccataataaaaaaaatctagcatgacagcttttccatagatttttaataatgtaaaagcagccgacttcaggcaatggatagtggggcccgtatcaacttcggacgctccacttgcaacggggtgggcccaatataacaacgacgtcgtaacagataaagcgaagcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacagatgcagatcttcgtcaagaccctcaccggcaagaccatcacccttgaggtggaaagctctgacaccatcgacaacgtcaaggccaagatccaggacaaggaaggaatccccccggaccagcaacgtctcattttcgccggaaagcaacttgaggacggccgtacccttgctgactacaacattcagaaggagagtactcttcacctcgtcctccgtctccgtggtggcatgcagatcttcgttaagacactcaccggcaagaccataaccctagaggttgaaagctccgacaccatcgataacgtcaaggccaagatccaggacaaggagggtatccccccggaccagcaacgtctcatcttcgccggaaagcagctcgaggacggccgcaccctcgccgactacaacatccagaaggaatcaacccttcacctcgtcctccgtctccgtggtggcatgcagatcttcgttaagaccctcaccggcaagactattaccctagaagtcgaaagctccgacaccatcgacaacgtcaaggctaagattcaggacaaggagggaatccccccagaccagcagaggctgatcttcgccgggaagcagctcgaggacggacgcacccttgctgactacaacatccagaaggagtcaactctccacttggtgttgcgtcttcgtggtggtatgcagattttcgtgaagactcttacgggtaagactattaccctcgaggtggagagctctgacaccattgacaatgtgaaggccaagattcaggacaaggaaggcatcccaccggaccagcagaggctgatttttgctggcaagcagctcgaggatggaaggaccctcgctgactacaacatccagaaggaatcaacccttcaccttgtcctccgtctccgtggggggtttgagggcagaggaagtcttctaacatgcggtgacgtggaggagaatcccggcccttctagaatggcccaagtgagcagagtgcacaatcttgctcaaagcactcaaatttttggccattcttccaactccaacaaactcaaatcggtgaattcggtttcattgaggccacgcctttggggggcctcaaaatctcgcatcccgatgcataaaaatggaagctttatgggaaattttaatgtggggaagggaaattccggcgtgtttaaggtttctgcatcggtcgccgccgcagagaagccgtcaacgtcgccggagatcgtgttggaacccatcaaagacttctcgggtaccatcacattgccagggtccaagtctctgtccaatcgaattttgcttcttgctgctctctctgaggtgaagtttatttatttatttatttgtttgtttgttgttgggtgtgggaataggagtttgatgtgtagagtggattttgaatatttgatttttttttgtattattctgtgaaaatgaagcatcatgtcccatgaaagaaatggacacgaaattaagtggcttatgatgtgaaatgaggatagaaatgtgtgtagggttttttaatgggtagcaataagcatattcaatatctggattgatttggacgtttctgtataaaggagtatgctagcaatgtgttaatgtatggcttgctaaaatactcctaaaaatcaagtgggagtagtatacatatctacagcaaatgtattaggtgaggcatttggcttctctattgtaaggaacaaataatatcagttaatgtgaaaatcaatggttgatattccaatacattcatgatgtgttatttatatgtacctaatattgactgttgtttttctccgcaatgaccaagattatttattttatcctctaaagtgactaattgagttgcttactttagagaagttggacccattaggtgagagcgtggggggaactaatcttgaatatacaatctgagtcttgattatccaagtatggttgtatgaacaatgttagctctagaagataaaccctcccccaaaacacaaattagaatgacatttcaagttccatgtatgtcactttcattctattatttttacaacttttagttacttaacagatgtcttgttcagcataaattataatttattctgtttttttttagggaacaactgttgtagacaacttgttgtatagtgaggatattcattacatgcttggtgcattaaggacccttggactgcgtgtggaagatgacaaaacaaccaaacaagcaattgttgaaggctgtgggggattgtttcccactagtaaggaatctaaagatgaaatcaatttattccttggaaatgctggtactgcaatgcgtcctttgacagcagctgtggttgctgcaggtggaaatgcaaggtctgttttttttttttttgttcagcataatctttgaattgttcctcgtataactaatcacaacagagtacgtgttcttcttcctgttataatctaaaaatctcatccagattagtcatcctttcttcttaaaaggaacctttaattatcaatgtatttatttaatatttaaattagcttgtcaaagtctagcatatacatattttgattatattctgagaaatgcacctgagggtgttcctcatgatctacttcaacctctgttattattagattttctatcatgattactggtttgagtctctaagtagaccatcttgatgttcaaaatatttcagctacgtacttgatggggtgccccgaatgagagagaggccaattggggatttggttgctggtcttaagcaacttggtgcagatgttgattgctttcttggcacaaactgtccacctgttcgtgtaaatgggaagggaggacttcctggcggaaaggtatggtttggatttcatttagaataaggtggagtaactttcctggatcaaaattctaatttaagaagcctccctgttttcctctctttagaataagactaagggtaggtttaggagttgggttttggagagaaatggaagggagagcaatttttttcttcttctaataaatattctttaatttgatacattttttaagtaaaagaatataaagatagattagcataacttaatgttttaatcttttatttatttttataaatattatatacctgtctatttaaaaatcaaatatttgtcctccattccctttcccttcaaaacctcagttccaaatataccgtagttgaattatattttggaaggcctattggttggagacttttccttttcagagattatccctcacctttattatagcctttctatttttaaacttcatatagacgccattcttgttttaaaaaacactaagttttcttttagttataccttcccctccttattctctccaaagtaaatattgtagcaagtgttggttgatccattgtgaatcttattatcctatttaacatgcaggtgaaactgtctggatcagttagcagtcaatacttgactgctttgcttatggcagctcctttagctcttggtgatgtggaaattgagattgttgataaactgatttctgttccatatgttgaaatgactctgaagttgatggagcgttttggagtttctgtggaacacagtggtaattgggataggttcttggtccatggaggtcaaaagtacaagtaggtttctatgttttagtgttacatcacttttagtatccaaaatgcaatgaaattaaaactcatgtttcctattaggtctcctggcaatgcttttgttgaaggtgatgcttcaagtgccagttatttactagctggtgcagcaattactggtgggactatcactgttaatggctgtggcacaagcagtttacaggtattcatgaatgacactatcttctattatcttttattttatagcttacactatcattcctaattcaacccccttcttttctctttcccctcactcccaaaatagagcttatcattgtatccttaatagctcttttatgtgctgtttcatctcttgagcagattctacatgattatcctttttagtgaacttttttgtcttttaaataattgatatcatcatttgaacttgcacatcatctctatccttcctcattttattatatttcattaagagtgataaaattacattgtcaagtaaagcaagttagcgtatcagataatgatgcttcgattgcagaattggtgaaagcttgcacaattcattagcttagtgacaattgtgatgtagatagccataatcttccatgcaacttattatgctttttgtctaaaacttcttttgcacttaaacatgaattagagagttaggatgaacaatagtaacagacacaaagcttatttctagaccacccagtggtgaagacgaagcagcatgagtgagattaatggcagcatctgattgaagcttcttttggtattattggagatgaaatcttgagccaaattagggcttttgcctcctcaatagccattggttcaaaattgctttcaataaaatgattacagaatcaaattcttaagaaagaccttcaagaacaacatcaagatgtttcctgtgcaaaacaggctcacctacaaaagagtgtctacaagcacctttacacttgcaaaaaattccttaactgaacaattgccaagagtcatgttgcaaacttgagattggagttgattagatttggtgtgtgtgagattctggaaatgagcatgaagttttttgcaaagctcataaaatgaatgcagccaagaatcctagataggattgaatttgagagagaggcttgaagtcaagataaccgcaactgatcttgctgttcccatgctgcataggactcgttgattctaccaagataacaatcttctgtggtgagaatatgagtgggaattgtcaggcaacaaagaaaatgaaggctatgagcctttgttaaggtgcagagagaaaacagaaaatgaagaacttgtgtattgaaggaattgaaaagccaattacaattgtgttgcagagagatatttatagttctgaagtgtataactaactaaacagaatgctaccaacctagaggcagttgagaattgctcccttattacaatactctcacagccttgatgataggcttgatttgctgccaccgaagaagaacattattgttggagagtttttcagtgattgcatcagagaaagttacaagtgtgattggagatgaaggacttgcggtcgtcgaaggttaagtgttaagtggtggaatgtgtatcggaggtggttgcctagcccaagaagctggttgcagtggaagcggatgcggcagaagccatgggttcagatcagaactatgttctagataccatattagggttagctttcaagagaaaacttatgaaactgaactgtaaactgttgtattattgattagctgatctagtagtgatggaagcttgcttgcgaagcttctatggaggctggatcattgagcttcaatgaggtccttcaatggtgatttttcaccatggagatgcagcggaagataaatgaagaggtgagaggaggcgtcatccactaggaaataaactatggaagaaggagcttcaccaccaagagagtgtcttggataagaagcttagagaggaagcttcaatggaggaatagaaagagagagggggaacaaaaaattgaaggaggaaaagagggagagaagttgaactttgaagtgtgtctcacaagactcattcattaaagttacaacaagtgttacacatgcttctatttatagtctaggtaacttccttgagaagctagaacttaactacacacacctctctaataactaagataacctccttgagaaatttccttgagaagcttccttaagaagattcctagagaagctaaagcttagttacacacacctctctaataactaagttcacctccttgagatgagaagctagagccttagctacacacacccttataatagctaaactcactctattccaaaatacatgaaaatacaaaaaagttcctactacaaagattactcaaaatgtcctgaaatacaagactaaaactctatactactagaatggtcaaaatacaaggtccaaaagaaggaaaaacctattctaatatttacaaagagagtggacccaaccttagtccatgggctcagaaatctaccctgaggttcatgggaatcctagggtcttctttagtagctctagtccaattttcttggagtcttctatccaatatccttgggggtaggattgcatcatgtagtacaagagaggggaatatatatacagttgtaagttgtaactacctaatcaaacccaactgactcaccatacacagaaaaacaatgtacaaaggataatagaaaaactgaaactgagatatactcattacatagaagaattacatgatgaatctgaaaattttaattgggaaactgaggacaataacaaggctttcagagttcattttgacaataggtaaattatggacttcaacttagacctcaagggtatgcttgggagtggggttttagaggaaaaggggaaggagggaaattttttaattctagtaaatattctctattttgattaatttttaaaataaaacaaataaacttatagattagcattcacttaatgtcctgatcttttatttgtttcatgaagttttttatagcctttctgtttctgttaaaagaaattggagggtgttcataatctatatgcccatatatatgtattgtcctgttgtctgtgtttctgttttctttctttagtgtctcgcctttccttaattccttatcaacaccttgtttatttttgaaaacttaaatgtcctcattttttttacctgtactataactttccaaacaaatagtagaaaatgtttcaaatattccccagaactgtttcagtgaacaaagttcaagccagaaaaaaagcaagcagaggctaaagaagtatatcttattgtgaagagtaatctcaattggttaattatcgaattgtatcattttacaatagaaaattagaagtgggttgattagtttatacgaacaattatttaaatagggttgatggtgtggttaattactgatcatatgttaagactatgagtcgatgaccagcctcagtgcagtcattttaagttccattattttttaattgtgcttatgtttaactttgtccttcttttaaagggagatgtaaaatttgctgaagttcttgaaaagatgggagctaaggttacatggtcagagaacagtgtcactgtttctggaccaccacgagatttttctggtcgaaaagtcttgcgaggcattgatgtcaatatgaacaagatgccagatgttgccatgacacttgctgttgttgcactatttgctaatggtcccactgctataagagatggtatggtttagctgtttgtttttgctcaagagcttgttttcacatatttgtggtcaagagcggtttagctgtttttttttttaaattgtggttaatgaaattattataattatactaatattgaaattggatcactaaccatgtgaaatgcttgaatttttagcttgaaaacttcttttgtggatgatttttcttttatctatgtagtgtggggcatctatgttgttcctttaagctttgtgtcatagttgtacaacctggtctgtgggctggtccagttcttactcatgactgtcttttgcagcaaactggttaaacttgggtgacctgttgacccacccagattggcctgttcttttttaaataaaactccaaaaggctacttataagatttcaacatgatacttgaatgaccacccttactttaaaggtcattatgccatcagaccccttgattctctgttcagttatgttcttattcttattattttttaatttatattattatccaacgatctgatctttgtacatgatgctggacaatcatcaaagctagtttggttacatcatataataccttttttttcataaattattttttcccataatatatttatatgtatttttttaatacaatattctggggaaattttttttttctgttttcttttttctggtttgaatactatttcaatgactgggccttattattttctggtttgggttatacaactgtcctttgatgtacttttgaatgcttcaatagtatttttcataatcatggaaggcactagagtgcagtgcatattgcacaaggttctttaatggcatcttgaatttgttagaacatggagttatgatatacaaccattcctatgtcttcatctaatagctaaagcttttggcgtagttggttaacatggtattggaatcttattttaaaggtccatcttattcgcaagtacaaggcaggtggacctgcgcattattcacgtgccaggctcaaagggattttgtatgagggggaatttttggaatataatatatagaatcattcatgtggttcacctatcatgtgtcaactcaagtttttgtcctagttgtattattgcagtgtctaaactaggcatgatcttctgtggaaggaaatttaacctaaggaacttgaatgcttcttgttctgagatctttattttttttctccaatctttttaatacttgaaaggttttagttgtatctataaagggtgcagaattagggggtcagcttttctgttgtaagcgtgaattaagtgcattttatgttgttccctattttatacatttgaactatcaaccatatcatttagcaaacttctttgttgctcagattcccatttattcttgtttcctctattttttagtggcaagttggagagttaaagagactgagaggatgatagcaatctgcacagaactcagaaaggtcttgctaattcctttatggtttctatattactggactttttacatctcactgacctactactgtcttggtatttcagctaggagcaacagttgaagaaggtcctgattactgtgtgattactccacctgagaaattgaatgtcacagctatagacacatatgatgaccacagaatggccatggcattctctcttgctgcttgtggggatgttccagtaaccatcaaggatcctggttgcaccaggaagacatttcctgactactttgaagtccttgagaggttaacaaagcactaa

SEQ ID NO: 9 - DNA, 5381 bp, Artificial Sequence
  pUbi3Ubi3T2AEPSPS chr 3 CDS
gtgaaaattttcacaagattttgaaagctctgaactcattagcacctgatttaatggttagaccaggctcaactgtgcctgcaaaaataagggaaagcacaaggttttatccttattttaaggtatgtgatcattatctaattataacaaaattataattataattgtgatggcatgtgcagcacttcataattttcttcgcaaagaatgtcgttctgatgaatttccagtggaacctactgacgagtcttcatcttcatcttcagtgttaccaaattacgaagacaatgatcatgaacccattgttcaaacacaagagcaggaacgagaagatgctaatatatggaggactaatataggttcagatatgtggagaaatgctaataattaggcgaacatgaagtgagaatcactttgttattatttttttaggcaataatgactttgttattaaaaggtttaaaatttctatcgtttttattttctttattcaaacattataatttatttatcatctttttcattcacttttgtaacttccgttatttttttgtttaaaatgtattaatctttcaaaatcttaaaaatccataaagtactttgaaatcttaaaaatctgtgttagaaatccattaaaatcttgggttaagaatcctgattgtaaaaagtcttttaaaaaaaatcttttaaaatcccacaaaatcaatacaatcccacataatcttttaaaatcttcaagattgtttttgtcaaaatattctctcaaaatcccaatccaatacaccccccttaattaacaaaaatgaatacgggctaacatggttatgggcttaggctgcctcattacccattacatgctctgttcctaaaaaaaagaagtttgggcctttattctccagtaagctttttaattgggctcaagaactttaggtatttaggtgagattcaatgaaaatttatacattaggcttgatttttttttttcaatttacgcaaaaatgaaagaaaaagtcacactctataaactttatttttttcaatattaatacattgttattgtggatatatcattagcggaaaagtaattctaataccagatttaattgactttgactaatttttttatgtgatcaaataatttttttagcgaaaaacttatatatatatatatatatatatatatatataagttttgttacttttccgcgtgatttttaatcaaactttggggataatttcccctccaattcagccaaaaaaaaaaaaaaactgacaccatatattattagtaggcaacttgttcgtaaatggtgtggcttatgcgaatggaaattggattcgttttctttaacatcattattgtttttgtcaatgagctatcttttagtcttatgttattggtgaatctgtccttaagttgcagcatttaacacatctcctcattagagaaaaaaattcttccctaaacgatagtaaaaacatctaataagaaataagaaagaaaaattaggaaaaagaaaagttcattaaaaaaatcttttggattatttttaaaaaaatatctaaatattttttaaatgaataattttatataaactgtaactaaaagtatacaagtaatgtatgttaacaaaatacttgaaaaatctactgaaaatatatcttacaaagtgaaattaaataagaaagaatttagtggaataattatgattttatttaaaaaataattattaaagatttttttgctccataataagaaaacttttcaattattcttttctggtccataataaaaaaaatctagcatgacagcttttccatagatttttaataatgtaaaagcagccgacttcaggcaatggatagtggggcccgtatcaacttcggacgctccacttgcaacggggtgggcccaatataacaacgacgtcgtaacagataaagcgaagcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacagatgcagatcttcgtcaagaccctcaccggcaagaccatcacccttgaggtggaaagctctgacaccatcgacaacgtcaaggccaagatccaggacaaggaaggaatccccccggaccagcaacgtctcattttcgccggaaagcaacttgaggacggccgtacccttgctgactacaacattcagaaggagagtactcttcacctcgtcctccgtctccgtggtggcatgcagatcttcgttaagacactcaccggcaagaccataaccctagaggttgaaagctccgacaccatcgataacgtcaaggccaagatccaggacaaggagggtatccccccggaccagcaacgtctcatcttcgccggaaagcagctcgaggacggccgcaccctcgccgactacaacatccagaaggaatcaacccttcacctcgtcctccgtctccgtggtggcatgcagatcttcgttaagaccctcaccggcaagactattaccctagaagtcgaaagctccgacaccatcgacaacgtcaaggctaagattcaggacaaggagggaatccccccagaccagcagaggctgatcttcgccgggaagcagctcgaggacggacgcacccttgctgactacaacatccagaaggagtcaactctccacttggtgttgcgtcttcgtggtggtatgcagattttcgtgaagactcttacgggtaagactattaccctcgaggtggagagctctgacaccattgacaatgtgaaggccaagattcaggacaaggaaggcatcccaccggaccagcagaggctgatttttgctggcaagcagctcgaggatggaaggaccctcgctgactacaacatccagaaggaatcaacccttcaccttgtcctccgtctccgtggggggtttgagggcagaggaagtcttctaacatgcggtgacgtggaggagaatcccggcccttctagaatggcccaagtgagcagagtgcacaatcttgctcaaagcactcaaattttcggtcattcttccaatcccaacgaacccaaatcggcgaattcggtttcattgaggccacgcctttggggtccctcgaaatctcgcatcttggtgcacaaaactggaagccttatgggaaattttaatgcggggaagggaaattccggcatgtttaaggtttctgcctccgtcgccgccgccgcagagaagccttcgacggcgccggagatcgtgttggaacctatcaaagacatctcgggtaccatcacattgccagggtctaagtctctgtccaatcgaattttgcttcttgctgctctctctgagggaacaactgttgtagacaacttgctgtacagcgaggatattcattacatgcttggtgcattaaggacccttggactgcgtgtggaagacgaccaaacaaccaaacaagcaattgtggaaggctgtgggggattgtttcccactattaaagaatctaaagatgaaatcaatttattccttggaaatgctggtactgcgatgcgtcctttgacagcagctgtagttgctgcaggtggaaatgcaagctacgtacttgatggagtgccccgaatgagagagaggccaattggggatttggttgctggtcttaagcagctcggtgcagatgttgattgctttcttggcacaaactgtccacctgttcgtgtaaatgggaagggaggacttcctggcggaaaggtgaaactgtctggatcaattagcagtcaatacctaactgctttgcttatggcagctcctttagctcttggcgacgtggaaattgagattgttgataaactgatttctgttccatatgttgaaatgactctgaagttgatggagcgttttggagtttctgtggaacacagtggtaattgggataagttcttggtccatggaggtcaaaagtacaagtctcctggcaatgcttttgttgaaggtgatgcttcaagtgccagttacttcctagctggtgcagcagttactggtgggactatcactgttaatggctgtggcacaaacagtttacagggagatgtaaaatttgctgaagttcttgaaaagatgggagctaaggttacatggtcagagaacagtgtcaccgttactggaccaccacaagattcttctggtcaaaaagtcttgcaaggcattgatgtcaatatgaacaagatgccagatgttgccatgactcttgccgttgtcgcactatttgctaatggtcaaactgccatcagagatgtggcaagttggagagttaaagagactgagaggatgatagcaatctgcacagaactcagaaagctaggagcaacagttgaagaaggtcctgattactgtgtgattactccacctgagaaattgaatgtcacagctatagacacatatgatgaccacagaatggccatggcattctctcttgctgcttgtggggatgttccagtaaccatcaaggatcctggttgcaccaggaagacatttcccgactactttgaagtccttgagaggttcacaaggcactaa

SEQ ID NO: 10 - DNA, 11334 bp, Artificial Sequence
  pUbi3Ubi3T2AEPSPS chr 3 genomic sequence
gtgaaaattttcacaagattttgaaagctctgaactcattagcacctgatttaatggttagaccaggctcaactgtgcctgcaaaaataagggaaagcacaaggttttatccttattttaaggtatgtgatcattatctaattataacaaaattataattataattgtgatggcatgtgcagcacttcataattttcttcgcaaagaatgtcgttctgatgaatttccagtggaacctactgacgagtcttcatcttcatcttcagtgttaccaaattacgaagacaatgatcatgaacccattgttcaaacacaagagcaggaacgagaagatgctaatatatggaggactaatataggttcagatatgtggagaaatgctaataattaggcgaacatgaagtgagaatcactttgttattatttttttaggcaataatgactttgttattaaaaggtttaaaatttctatcgtttttattttctttattcaaacattataatttatttatcatctttttcattcacttttgtaacttccgttatttttttgtttaaaatgtattaatctttcaaaatcttaaaaatccataaagtactttgaaatcttaaaaatctgtgttagaaatccattaaaatcttgggttaagaatcctgattgtaaaaagtcttttaaaaaaaatcttttaaaatcccacaaaatcaatacaatcccacataatcttttaaaatcttcaagattgtttttgtcaaaatattctctcaaaatcccaatccaatacaccccccttaattaacaaaaatgaatacgggctaacatggttatgggcttaggctgcctcattacccattacatgctctgttcctaaaaaaaagaagtttgggcctttattctccagtaagctttttaattgggctcaagaactttaggtatttaggtgagattcaatgaaaatttatacattaggcttgatttttttttttcaatttacgcaaaaatgaaagaaaaagtcacactctataaactttatttttttcaatattaatacattgttattgtggatatatcattagcggaaaagtaattctaataccagatttaattgactttgactaatttttttatgtgatcaaataatttttttagcgaaaaacttatatatatatatatatatatatatatatataagttttgttacttttccgcgtgatttttaatcaaactttggggataatttcccctccaattcagccaaaaaaaaaaaaaaactgacaccatatattattagtaggcaacttgttcgtaaatggtgtggcttatgcgaatggaaattggattcgttttctttaacatcattattgtttttgtcaatgagctatcttttagtcttatgttattggtgaatctgtccttaagttgcagcatttaacacatctcctcattagagaaaaaaattcttccctaaacgatagtaaaaacatctaataagaaataagaaagaaaaattaggaaaaagaaaagttcattaaaaaaatcttttggattatttttaaaaaaatatctaaatattttttaaatgaataattttatataaactgtaactaaaagtatacaagtaatgtatgttaacaaaatacttgaaaaatctactgaaaatatatcttacaaagtgaaattaaataagaaagaatttagtggaataattatgattttatttaaaaaataattattaaagatttttttgctccataataagaaaacttttcaattattcttttctggtccataataaaaaaaatctagcatgacagcttttccatagatttttaataatgtaaaagcagccgacttcaggcaatggatagtggggcccgtatcaacttcggacgctccacttgcaacggggtgggcccaatataacaacgacgtcgtaacagataaagcgaagcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacagatgcagatcttcgtcaagaccctcaccggcaagaccatcacccttgaggtggaaagctctgacaccatcgacaacgtcaaggccaagatccaggacaaggaaggaatccccccggaccagcaacgtctcattttcgccggaaagcaacttgaggacggccgtacccttgctgactacaacattcagaaggagagtactcttcacctcgtcctccgtctccgtggtggcatgcagatcttcgttaagacactcaccggcaagaccataaccctagaggttgaaagctccgacaccatcgataacgtcaaggccaagatccaggacaaggagggtatccccccggaccagcaacgtctcatcttcgccggaaagcagctcgaggacggccgcaccctcgccgactacaacatccagaaggaatcaacccttcacctcgtcctccgtctccgtggtggcatgcagatcttcgttaagaccctcaccggcaagactattaccctagaagtcgaaagctccgacaccatcgacaacgtcaaggctaagattcaggacaaggagggaatccccccagaccagcagaggctgatcttcgccgggaagcagctcgaggacggacgcacccttgctgactacaacatccagaaggagtcaactctccacttggtgttgcgtcttcgtggtggtatgcagattttcgtgaagactcttacgggtaagactattaccctcgaggtggagagctctgacaccattgacaatgtgaaggccaagattcaggacaaggaaggcatcccaccggaccagcagaggctgatttttgctggcaagcagctcgaggatggaaggaccctcgctgactacaacatccagaaggaatcaacccttcaccttgtcctccgtctccgtggggggtttgagggcagaggaagtcttctaacatgcggtgacgtggaggagaatcccggcccttctagaatggcccaagtgagcagagtgcacaatcttgctcaaagcactcaaattttcggtcattcttccaatcccaacgaacccaaatcggcgaattcggtttcattgaggccacgcctttggggtccctcgaaatctcgcatcttggtgcacaaaactggaagccttatgggaaattttaatgcggggaagggaaattccggcatgtttaaggtttctgcctccgtcgccgccgccgcagagaagccttcgacggcgccggagatcgtgttggaacctatcaaagacatctcgggtaccatcacattgccagggtctaagtctctgtccaatcgaattttgcttcttgctgctctctctgaggtaaagtttatttatttattttttctgtatccaaaaatgtaaattgttagtttgttaatttttgttagcagtgagagtcgaactacttggcttctttcttagtcatccaaccaaccttatatctccaaaatttatttattggttttttttggggttatgtggttaggaataggagtttgatgcgtggagtggattttgaatatttgattttttttttgtattattcagtgaaaatgaagcatcttgtcccatgaaagaaatggacacgaaattaagtggcgtatgatttagaatgatgatagaaatgtgtataggtggttttaatgtgtagcaataagcatattcaatatctggattgatttggacgtttctgtataaaggagtatgctagcaatgtgttaataatgtcttgttaaaagtatggatggctaaaataatcataaaaatcgagtgggagtagtatacatatctacaggaaatgtattaggtgaggcatttggcttctctattgcgagtaaggaacaagtaatctcagttaatgtgaaaatcaatggttgatattccaatacattcatgatgtgttatttacgggaacgcaatattgactgttgattttatctgcagctgagattatatactatatccttccaaagaaatctttgactcttgattatccaagtatggttgtattaccaattttagctctagaagataatccctcccccaaaacacaaattagaatggtgctgcaagttctgtgttactttcattctattatttttataacttttaattatttaatagatgtcttgtttggcataaactataatttattctgtttttttttatttacttatttagggaacaactgttgtagacaacttgctgtacagcgaggatattcattacatgcttggtgcattaaggacccttggactgcgtgtggaagacgaccaaacaaccaaacaagcaattgtggaaggctgtgggggattgtttcccactattaaagaatctaaagatgaaatcaatttattccttggaaatgctggtactgcgatgcgtcctttgacagcagctgtagttgctgcaggtggaaatgcaaggtctgttttttgttttgtttgttcatcatgatctctgaattgttcctcgtataactaatcacatcagactatgtgttcttcctccatcctgttataatctaaaaatctaatccagattagtcatccttctttaaatgaacctttaattatatctatgtatttatttaacatgtaaattagcttgtcaagtcaaagtctagcatatagatatactgattacactctgaggaatgcacctgagggtcttactcatgatctacttcaaccttgccactttcttcttttattattagatcacctatcatgattactggtttgagtctctaaatagaccatcttgatgttcaaaatatttcagctacgtacttgatggagtgccccgaatgagagagaggccaattggggatttggttgctggtcttaagcagctcggtgcagatgttgattgctttcttggcacaaactgtccacctgttcgtgtaaatgggaagggaggacttcctggcggaaaggtatggtttggatttccattagaataaggtggactaactttcctggagcaaaattctaatttaaaccctgtttccctctctttagaataagacactaagggtatgtttaggagttgggttttggcgagaaagggaagggagggcaattttttttctaataaatattctttaatttgataaaatttttaaacgaaggaatatgaagatagattagcataacttaatgttttaatcttttatttatttttataaatattatatacctctatttaaaaacaagatatttttcctccattccctttccctttgaaacctcagttccaaatataccgtacttgaattatattttggaaggtgtattggttggagacctttccttttcagaggttatccctcacctttattatagcctttctactctctcaatgagttcattgtgcattgagtcattgaacttcctgtgaaaagaaattgtttacttttctttatttgttacctacatccttattcctgttttaaaaaatactaagttttcttttagttatgccttcccctccttattctctccaaagaaaatataatagcgaatgttggtttatccattgtgaatcttattatcctatttcacatgcaggtgaaactgtctggatcaattagcagtcaatacctaactgctttgcttatggcagctcctttagctcttggcgacgtggaaattgagattgttgataaactgatttctgttccatatgttgaaatgactctgaagttgatggagcgttttggagtttctgtggaacacagtggtaattgggataagttcttggtccatggaggtcaaaagtacaagtaggtttctatgttttagcattacatcactttttagtatccaaaatgcaatgaaatcaaaactcatgttttctatcaggtctcctggcaatgcttttgttgaaggtgatgcttcaagtgccagttacttcctagctggtgcagcagttactggtgggactatcactgttaatggctgtggcacaaacagtttacaggtattcatgcttgacactattttctattatcttttattttatggtctatactgtatcattcctaattcaacccccttcttttctctttcccctcacttccaaaaaaatagagcttatcattgtattcttaatagctcttgtatgtcctttttcgtctcttgagcagattctaccgtgattatcctttttagtaaacttttttttgtgtgtcttttaaataattgatatcatcatgtgaactcacacatcatctctatcccttcctcattttattatatttcattaacagtgataaagataaaaataaattgtcaagtaaagcaagttggcatgtcagataatgatgcttcaattgcaaaattggtgaaagcttgcacagtacattagctttggtgacaattgtgatgtagatagccataacctcccatgcaacttattatgttgtttgcttaaacttcttttgcacttaaacacggattagagagagtgaggatgaacaataataacagacacaaagcttagttttataccacccggcacccagtggtgaggaccaagcagcatgagtgagatcaatggcagcatctgattgaagcttcttgtggtatttttggagatgaaatcttgaggcgaattagggcatctggcttaacaatagccattggttcaaatttgcttttaataaaattgattacagaatcaaattcttgaggaagccttcaagaactacatcatgatgttccctgtgcaaaacaggttcgccgacaaaagagaacatctacaagttcctttacacatgccaaaaattcctcaactgaacaattgccaagagtcatgttgcgaatttcaggttggagttgacgaaatttggtgcgtgtatgattctggaaatgagcatgaagttttttcctaagctcataaaagtgaatgcagccaagaaccctagataggattgaatcggagagaggcttgaagccaagataacagcaactgatcttgctgttcccatgctgcgtaggactcattgattctaccaagatcacaatcttctgtggtgagaaaatggggtgaattgtcaaccaatagagaaaatggtgaaggctgagtcttgatgataggcttgatttgcttgcgccgaagaagaacgttattgttgtagagtttctcagtgattgtatgagagtaagttacaggtgcgattggagatgacggacttgtggtgggaggagggtaagtgttaagtggtggaatgtgtatcagaggtggttgcccagccaaagaagctggttgcggtggtgcagtggaagccatgggttttgatgcttttgagagaaaacatatgaaactgaactgtaaacttttgtattattgattagctgatttggtagtacaagagaggaatatatatacagttttaatgtaactacctaaccaaacatgaggaactctccatacacagaaagacgtatacaaaggataacagacaaacagaaacttagatatactcatacagagaagaattacaggatgaatctgagaattttaattgggaaactgaggaaaatatcaagggtttcagaattcattttgacgataggcaaatcatggacttaacttagacctcaagggtacgcttgggagtgggggtttttagagggaaaagggaaggagggcaaacttttaattcctaataaatattctcttttttgatttttttttaaaataaaacaaataaacttatagattagcatacacttaatgtcctaatcttttatttatttgcatgagtttttttcttttatattctttctgtttctgttttctttctttagtgtctcttgtctttccttaattccttatcaataccttgtttatttttgaaaacttaattttcctcatttttttaacctataggctatactatagtatgtataactattcaaataaatagtagaaaatgtttcaaatattccccagaactgtttcagtgaacaaagttcaagccagaaaaaagaagtatatcttattgtgaagagtagtctcaagtggttaattgtcaaattgtatcattttagaatacaaaattagaagtggtttgattagtttatacgaacgattatttaaatagggttgatgatgtggttaattactgatcatatgtgaagactgtgagtcgatgaccagcccagtgcagtcattttaaattccattattttttaatttgtgcttatgttaaactttgtccttctttcaaagggagatgtaaaatttgctgaagttcttgaaaagatgggagctaaggttacatggtcagagaacagtgtcaccgttactggaccaccacaagattcttctggtcaaaaagtcttgcaaggcattgatgtcaatatgaacaagatgccagatgttgccatgactcttgccgttgtcgcactatttgctaatggtcaaactgccatcagagatggtatggtttagctgtttgtttgtgttcaagagcttgttttaacaaattgttttttttttaattatggttaatgaaattattataattacactaatattgaaattggatcactaaccatgtgaaatgcttgaatttttagcttgaaaacttcttttgtggacaatttttcttttatctatggattgtggggcatctatgttgttcctttaagcttcgtacaacctggtctgtgggctggtccagtccttcttactcatgacagtcttttgcagtaaactggttaaacttgggtgacctgttgacccacctagattggcctgttctttttaaataaaacttcaaaaggctacttataagatttcaacatgatacttgaatgatcacccttactctaaaggccattatctgttcagttatgttcttcttcttcttcttattatttttaatttatattattatccaacgatctgatctttgaacatgatgctggacaatcatcaaagctagtttggttacatcatataataccttttttttcatatattatttttcccataatatatttttgtgtttttttttcatacaatattattctgaaaaaaacgtgtttcgtttttttcctggcttgaatgctatttcaatgactgggccttattattttctggtttaggttatacaactgtcctccaattgacttttgaaggcttcaatagttttcttcttaatcatggaaggcactagagtgcatattgaatatgttagaacatggaaggcttaaatatctttttagtctttgcaatttaacgtttttttgcttttagtccttgcaaaattataatttttttttagtcctggctaattatgtttgttttgttttttgtccttatatgctttagataacactttttttgttactgttcaaagcactatctaaagtgctttaaggactaaacacaaaacaaacataatttgcaaggactgaaaaacaaaaaaataattttgcaaggacgaaaaaataaaaataaaaaacacaaaattgcaagaccaaaaatatatttaaaccaacatgaaattatggtataaaaccattcatacgtcttcatctaatagcttaagcttttgggatagttggttaacaatatggtattagagtcttatgatttttaggtggtctttgagttcaatccttgctgctcccagttctagttaatttctttttggtccacttcaaataaaaagttgaatttaagggtaaggcagctggacctgcacattaatcatgtgtcaggctcaaagggcttttgtatggggggcatgtttggaatataatatagaatcattcatgtggtttcacctatcaaggatcaactctagtttttgtcatacttgtattattacagtgtctaaactagacatgatctttttccggaaggaaatttaacttaaaaggaacttagatgcttcttgttctgtgatctttatttgtttattttttccaatctttttaacaattgaaaggttttagttgtatccaatctttttatacatttggactatctaccatatcatttagcaaacttgtttgctgctcagattctcctttggactatctaccatatcatttagcaaacctgttcactgctcagattctcatttattctttgtttcctctatttttcagtggcaagttggagagttaaagagactgagaggatgatagcaatctgcacagaactcagaaaggtgagtgtcttgctaattcctttatggtttcgatattactgaactttttacatctcactgacctactctgtcttggtatttcagctaggagcaacagttgaagaaggtcctgattactgtgtgattactccacctgagaaattgaatgtcacagctatagacacatatgatgaccacagaatggccatggcattctctcttgctgcttgtggggatgttccagtaaccatcaaggatcctggttgcaccaggaagacatttcccgactactttgaagtccttgagaggttcacaaggcactaa

SEQ ID NO: 11 - DNA, 4403 bp, Artificial Sequence
  pUbi3EPSPS chr 1 CDS
gtgaaaattttcacaagattttgaaagctctgaactcattagcacctgatttaatggttagaccaggctcaactgtgcctgcaaaaataagggaaagcacaaggttttatccttattttaaggtatgtgatcattatctaattataacaaaattataattataattgtgatggcatgtgcagcacttcataattttcttcgcaaagaatgtcgttctgatgaatttccagtggaacctactgacgagtcttcatcttcatcttcagtgttaccaaattacgaagacaatgatcatgaacccattgttcaaacacaagagcaggaacgagaagatgctaatatatggaggactaatataggttcagatatgtggagaaatgctaataattaggcgaacatgaagtgagaatcactttgttattatttttttaggcaataatgactttgttattaaaaggtttaaaatttctatcgtttttattttctttattcaaacattataatttatttatcatctttttcattcacttttgtaacttccgttatttttttgtttaaaatgtattaatctttcaaaatcttaaaaatccataaagtactttgaaatcttaaaaatctgtgttagaaatccattaaaatcttgggttaagaatcctgattgtaaaaagtcttttaaaaaaaatcttttaaaatcccacaaaatcaatacaatcccacataatcttttaaaatcttcaagattgtttttgtcaaaatattctctcaaaatcccaatccaatacaccccccttaattaacaaaaatgaatacgggctaacatggttatgggcttaggctgcctcattacccattacatgctctgttcctaaaaaaaagaagtttgggcctttattctccagtaagctttttaattgggctcaagaactttaggtatttaggtgagattcaatgaaaatttatacattaggcttgatttttttttttcaatttacgcaaaaatgaaagaaaaagtcacactctataaactttatttttttcaatattaatacattgttattgtggatatatcattagcggaaaagtaattctaataccagatttaattgactttgactaatttttttatgtgatcaaataatttttttagcgaaaaacttatatatatatatatatatatatatatatataagttttgttacttttccgcgtgatttttaatcaaactttggggataatttcccctccaattcagccaaaaaaaaaaaaaaactgacaccatatattattagtaggcaacttgttcgtaaatggtgtggcttatgcgaatggaaattggattcgttttctttaacatcattattgtttttgtcaatgagctatcttttagtcttatgttattggtgaatctgtccttaagttgcagcatttaacacatctcctcattagagaaaaaaattcttccctaaacgatagtaaaaacatctaataagaaataagaaagaaaaattaggaaaaagaaaagttcattaaaaaaatcttttggattatttttaaaaaaatatctaaatattttttaaatgaataattttatataaactgtaactaaaagtatacaagtaatgtatgttaacaaaatacttgaaaaatctactgaaaatatatcttacaaagtgaaattaaataagaaagaatttagtggaataattatgattttatttaaaaaataattattaaagatttttttgctccataataagaaaacttttcaattattcttttctggtccataataaaaaaaatctagcatgacagcttttccatagatttttaataatgtaaaagcagccgacttcaggcaatggatagtggggcccgtatcaacttcggacgctccacttgcaacggggtgggcccaatataacaacgacgtcgtaacagataaagcgaagcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacagatggcccaagtgagcagagtgcacaatcttgctcaaagcactcaaatttttggccattcttccaactccaacaaactcaaatcggtgaattcggtttcattgaggccacgcctttggggggcctcaaaatctcgcatcccgatgcataaaaatggaagctttatgggaaattttaatgtggggaagggaaattccggcgtgtttaaggtttctgcatcggtcgccgccgcagagaagccgtcaacgtcgccggagatcgtgttggaacccatcaaagacttctcgggtaccatcacattgccagggtccaagtctctgtccaatcgaattttgcttcttgctgctctctctgagggaacaactgttgtagacaacttgttgtatagtgaggatattcattacatgcttggtgcattaaggacccttggactgcgtgtggaagatgacaaaacaaccaaacaagcaattgttgaaggctgtgggggattgtttcccactagtaaggaatctaaagatgaaatcaatttattccttggaaatgctggtactgcaatgcgtcctttgacagcagctgtggttgctgcaggtggaaatgcaagctacgtacttgatggggtgccccgaatgagagagaggccaattggggatttggttgctggtcttaagcaacttggtgcagatgttgattgctttcttggcacaaactgtccacctgttcgtgtaaatgggaagggaggacttcctggcggaaaggtgaaactgtctggatcagttagcagtcaatacttgactgctttgcttatggcagctcctttagctcttggtgatgtggaaattgagattgttgataaactgatttctgttccatatgttgaaatgactctgaagttgatggagcgttttggagtttctgtggaacacagtggtaattgggataggttcttggtccatggaggtcaaaagtacaagtctcctggcaatgcttttgttgaaggtgatgcttcaagtgccagttatttactagctggtgcagcaattactggtgggactatcactgttaatggctgtggcacaagcagtttacagggagatgtaaaatttgctgaagttcttgaaaagatgggagctaaggttacatggtcagagaacagtgtcactgtttctggaccaccacgagatttttctggtcgaaaagtcttgcgaggcattgatgtcaatatgaacaagatgccagatgttgccatgacacttgctgttgttgcactatttgctaatggtcccactgctataagagatgtggcaagttggagagttaaagagactgagaggatgatagcaatctgcacagaactcagaaagctaggagcaacagttgaagaaggtcctgattactgtgtgattactccacctgagaaattgaatgtcacagctatagacacatatgatgaccacagaatggccatggcattctctcttgctgcttgtggggatgttccagtaaccatcaaggatcctggttgcaccaggaagacatttcctgactactttgaagtccttgagaggttaacaaagcactaa

SEQ ID NO: 12 - DNA, 11043 bp, Artificial Sequence
  pUbi3EPSPS chr 1 genomic sequence
gtgaaaattttcacaagattttgaaagctctgaactcattagcacctgatttaatggttagaccaggctcaactgtgcctgcaaaaataagggaaagcacaaggttttatccttattttaaggtatgtgatcattatctaattataacaaaattataattataattgtgatggcatgtgcagcacttcataattttcttcgcaaagaatgtcgttctgatgaatttccagtggaacctactgacgagtcttcatcttcatcttcagtgttaccaaattacgaagacaatgatcatgaacccattgttcaaacacaagagcaggaacgagaagatgctaatatatggaggactaatataggttcagatatgtggagaaatgctaataattaggcgaacatgaagtgagaatcactttgttattatttttttaggcaataatgactttgttattaaaaggtttaaaatttctatcgtttttattttctttattcaaacattataatttatttatcatctttttcattcacttttgtaacttccgttatttttttgtttaaaatgtattaatctttcaaaatcttaaaaatccataaagtactttgaaatcttaaaaatctgtgttagaaatccattaaaatcttgggttaagaatcctgattgtaaaaagtcttttaaaaaaaatcttttaaaatcccacaaaatcaatacaatcccacataatcttttaaaatcttcaagattgtttttgtcaaaatattctctcaaaatcccaatccaatacaccccccttaattaacaaaaatgaatacgggctaacatggttatgggcttaggctgcctcattacccattacatgctctgttcctaaaaaaaagaagtttgggcctttattctccagtaagctttttaattgggctcaagaactttaggtatttaggtgagattcaatgaaaatttatacattaggcttgatttttttttttcaatttacgcaaaaatgaaagaaaaagtcacactctataaactttatttttttcaatattaatacattgttattgtggatatatcattagcggaaaagtaattctaataccagatttaattgactttgactaatttttttatgtgatcaaataatttttttagcgaaaaacttatatatatatatatatatatatatatatataagttttgttacttttccgcgtgatttttaatcaaactttggggataatttcccctccaattcagccaaaaaaaaaaaaaaactgacaccatatattattagtaggcaacttgttcgtaaatggtgtggcttatgcgaatggaaattggattcgttttctttaacatcattattgtttttgtcaatgagctatcttttagtcttatgttattggtgaatctgtccttaagttgcagcatttaacacatctcctcattagagaaaaaaattcttccctaaacgatagtaaaaacatctaataagaaataagaaagaaaaattaggaaaaagaaaagttcattaaaaaaatcttttggattatttttaaaaaaatatctaaatattttttaaatgaataattttatataaactgtaactaaaagtatacaagtaatgtatgttaacaaaatacttgaaaaatctactgaaaatatatcttacaaagtgaaattaaataagaaagaatttagtggaataattatgattttatttaaaaaataattattaaagatttttttgctccataataagaaaacttttcaattattcttttctggtccataataaaaaaaatctagcatgacagcttttccatagatttttaataatgtaaaagcagccgacttcaggcaatggatagtggggcccgtatcaacttcggacgctccacttgcaacggggtgggcccaatataacaacgacgtcgtaacagataaagcgaagcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacagatggcccaagtgagcagagtgcacaatcttgctcaaagcactcaaatttttggccattcttccaactccaacaaactcaaatcggtgaattcggtttcattgaggccacgcctttggggggcctcaaaatctcgcatcccgatgcataaaaatggaagctttatgggaaattttaatgtggggaagggaaattccggcgtgtttaaggtttctgcatcggtcgccgccgcagagaagccgtcaacgtcgccggagatcgtgttggaacccatcaaagacttctcgggtaccatcacattgccagggtccaagtctctgtccaatcgaattttgcttcttgctgctctctctgaggtgaagtttatttatttatttatttgtttgtttgttgttgggtgtgggaataggagtttgatgtgtagagtggattttgaatatttgatttttttttgtattattctgtgaaaatgaagcatcatgtcccatgaaagaaatggacacgaaattaagtggcttatgatgtgaaatgaggatagaaatgtgtgtagggttttttaatgggtagcaataagcatattcaatatctggattgatttggacgtttctgtataaaggagtatgctagcaatgtgttaatgtatggcttgctaaaatactcctaaaaatcaagtgggagtagtatacatatctacagcaaatgtattaggtgaggcatttggcttctctattgtaaggaacaaataatatcagttaatgtgaaaatcaatggttgatattccaatacattcatgatgtgttatttatatgtacctaatattgactgttgtttttctccgcaatgaccaagattatttattttatcctctaaagtgactaattgagttgcttactttagagaagttggacccattaggtgagagcgtggggggaactaatcttgaatatacaatctgagtcttgattatccaagtatggttgtatgaacaatgttagctctagaagataaaccctcccccaaaacacaaattagaatgacatttcaagttccatgtatgtcactttcattctattatttttacaacttttagttacttaacagatgtcttgttcagcataaattataatttattctgtttttttttagggaacaactgttgtagacaacttgttgtatagtgaggatattcattacatgcttggtgcattaaggacccttggactgcgtgtggaagatgacaaaacaaccaaacaagcaattgttgaaggctgtgggggattgtttcccactagtaaggaatctaaagatgaaatcaatttattccttggaaatgctggtactgcaatgcgtcctttgacagcagctgtggttgctgcaggtggaaatgcaaggtctgttttttttttttttgttcagcataatctttgaattgttcctcgtataactaatcacaacagagtacgtgttcttcttcctgttataatctaaaaatctcatccagattagtcatcctttcttcttaaaaggaacctttaattatcaatgtatttatttaatatttaaattagcttgtcaaagtctagcatatacatattttgattatattctgagaaatgcacctgagggtgttcctcatgatctacttcaacctctgttattattagattttctatcatgattactggtttgagtctctaagtagaccatcttgatgttcaaaatatttcagctacgtacttgatggggtgccccgaatgagagagaggccaattggggatttggttgctggtcttaagcaacttggtgcagatgttgattgctttcttggcacaaactgtccacctgttcgtgtaaatgggaagggaggacttcctggcggaaaggtatggtttggatttcatttagaataaggtggagtaactttcctggatcaaaattctaatttaagaagcctccctgttttcctctctttagaataagactaagggtaggtttaggagttgggttttggagagaaatggaagggagagcaatttttttcttcttctaataaatattctttaatttgatacattttttaagtaaaagaatataaagatagattagcataacttaatgttttaatcttttatttatttttataaatattatatacctgtctatttaaaaatcaaatatttgtcctccattccctttcccttcaaaacctcagttccaaatataccgtagttgaattatattttggaaggcctattggttggagacttttccttttcagagattatccctcacctttattatagcctttctatttttaaacttcatatagacgccattcttgttttaaaaaacactaagttttcttttagttataccttcccctccttattctctccaaagtaaatattgtagcaagtgttggttgatccattgtgaatcttattatcctatttaacatgcaggtgaaactgtctggatcagttagcagtcaatacttgactgctttgcttatggcagctcctttagctcttggtgatgtggaaattgagattgttgataaactgatttctgttccatatgttgaaatgactctgaagttgatggagcgttttggagtttctgtggaacacagtggtaattgggataggttcttggtccatggaggtcaaaagtacaagtaggtttctatgttttagtgttacatcacttttagtatccaaaatgcaatgaaattaaaactcatgtttcctattaggtctcctggcaatgcttttgttgaaggtgatgcttcaagtgccagttatttactagctggtgcagcaattactggtgggactatcactgttaatggctgtggcacaagcagtttacaggtattcatgaatgacactatcttctattatcttttattttatagcttacactatcattcctaattcaacccccttcttttctctttcccctcactcccaaaatagagcttatcattgtatccttaatagctcttttatgtgctgtttcatctcttgagcagattctacatgattatcctttttagtgaacttttttgtcttttaaataattgatatcatcatttgaacttgcacatcatctctatccttcctcattttattatatttcattaagagtgataaaattacattgtcaagtaaagcaagttagcgtatcagataatgatgcttcgattgcagaattggtgaaagcttgcacaattcattagcttagtgacaattgtgatgtagatagccataatcttccatgcaacttattatgctttttgtctaaaacttcttttgcacttaaacatgaattagagagttaggatgaacaatagtaacagacacaaagcttatttctagaccacccagtggtgaagacgaagcagcatgagtgagattaatggcagcatctgattgaagcttcttttggtattattggagatgaaatcttgagccaaattagggcttttgcctcctcaatagccattggttcaaaattgctttcaataaaatgattacagaatcaaattcttaagaaagaccttcaagaacaacatcaagatgtttcctgtgcaaaacaggctcacctacaaaagagtgtctacaagcacctttacacttgcaaaaaattccttaactgaacaattgccaagagtcatgttgcaaacttgagattggagttgattagatttggtgtgtgtgagattctggaaatgagcatgaagttttttgcaaagctcataaaatgaatgcagccaagaatcctagataggattgaatttgagagagaggcttgaagtcaagataaccgcaactgatcttgctgttcccatgctgcataggactcgttgattctaccaagataacaatcttctgtggtgagaatatgagtgggaattgtcaggcaacaaagaaaatgaaggctatgagcctttgttaaggtgcagagagaaaacagaaaatgaagaacttgtgtattgaaggaattgaaaagccaattacaattgtgttgcagagagatatttatagttctgaagtgtataactaactaaacagaatgctaccaacctagaggcagttgagaattgctcccttattacaatactctcacagccttgatgataggcttgatttgctgccaccgaagaagaacattattgttggagagtttttcagtgattgcatcagagaaagttacaagtgtgattggagatgaaggacttgcggtcgtcgaaggttaagtgttaagtggtggaatgtgtatcggaggtggttgcctagcccaagaagctggttgcagtggaagcggatgcggcagaagccatgggttcagatcagaactatgttctagataccatattagggttagctttcaagagaaaacttatgaaactgaactgtaaactgttgtattattgattagctgatctagtagtgatggaagcttgcttgcgaagcttctatggaggctggatcattgagcttcaatgaggtccttcaatggtgatttttcaccatggagatgcagcggaagataaatgaagaggtgagaggaggcgtcatccactaggaaataaactatggaagaaggagcttcaccaccaagagagtgtcttggataagaagcttagagaggaagcttcaatggaggaatagaaagagagagggggaacaaaaaattgaaggaggaaaagagggagagaagttgaactttgaagtgtgtctcacaagactcattcattaaagttacaacaagtgttacacatgcttctatttatagtctaggtaacttccttgagaagctagaacttaactacacacacctctctaataactaagataacctccttgagaaatttccttgagaagcttccttaagaagattcctagagaagctaaagcttagttacacacacctctctaataactaagttcacctccttgagatgagaagctagagccttagctacacacacccttataatagctaaactcactctattccaaaatacatgaaaatacaaaaaagttcctactacaaagattactcaaaatgtcctgaaatacaagactaaaactctatactactagaatggtcaaaatacaaggtccaaaagaaggaaaaacctattctaatatttacaaagagagtggacccaaccttagtccatgggctcagaaatctaccctgaggttcatgggaatcctagggtcttctttagtagctctagtccaattttcttggagtcttctatccaatatccttgggggtaggattgcatcatgtagtacaagagaggggaatatatatacagttgtaagttgtaactacctaatcaaacccaactgactcaccatacacagaaaaacaatgtacaaaggataatagaaaaactgaaactgagatatactcattacatagaagaattacatgatgaatctgaaaattttaattgggaaactgaggacaataacaaggctttcagagttcattttgacaataggtaaattatggacttcaacttagacctcaagggtatgcttgggagtggggttttagaggaaaaggggaaggagggaaattttttaattctagtaaatattctctattttgattaatttttaaaataaaacaaataaacttatagattagcattcacttaatgtcctgatcttttatttgtttcatgaagttttttatagcctttctgtttctgttaaaagaaattggagggtgttcataatctatatgcccatatatatgtattgtcctgttgtctgtgtttctgttttctttctttagtgtctcgcctttccttaattccttatcaacaccttgtttatttttgaaaacttaaatgtcctcattttttttacctgtactataactttccaaacaaatagtagaaaatgtttcaaatattccccagaactgtttcagtgaacaaagttcaagccagaaaaaaagcaagcagaggctaaagaagtatatcttattgtgaagagtaatctcaattggttaattatcgaattgtatcattttacaatagaaaattagaagtgggttgattagtttatacgaacaattatttaaatagggttgatggtgtggttaattactgatcatatgttaagactatgagtcgatgaccagcctcagtgcagtcattttaagttccattattttttaattgtgcttatgtttaactttgtccttcttttaaagggagatgtaaaatttgctgaagttcttgaaaagatgggagctaaggttacatggtcagagaacagtgtcactgtttctggaccaccacgagatttttctggtcgaaaagtcttgcgaggcattgatgtcaatatgaacaagatgccagatgttgccatgacacttgctgttgttgcactatttgctaatggtcccactgctataagagatggtatggtttagctgtttgtttttgctcaagagcttgttttcacatatttgtggtcaagagcggtttagctgtttttttttttaaattgtggttaatgaaattattataattatactaatattgaaattggatcactaaccatgtgaaatgcttgaatttttagcttgaaaacttcttttgtggatgatttttcttttatctatgtagtgtggggcatctatgttgttcctttaagctttgtgtcatagttgtacaacctggtctgtgggctggtccagttcttactcatgactgtcttttgcagcaaactggttaaacttgggtgacctgttgacccacccagattggcctgttcttttttaaataaaactccaaaaggctacttataagatttcaacatgatacttgaatgaccacccttactttaaaggtcattatgccatcagaccccttgattctctgttcagttatgttcttattcttattattttttaatttatattattatccaacgatctgatctttgtacatgatgctggacaatcatcaaagctagtttggttacatcatataataccttttttttcataaattattttttcccataatatatttatatgtatttttttaatacaatattctggggaaattttttttttctgttttcttttttctggtttgaatactatttcaatgactgggccttattattttctggtttgggttatacaactgtcctttgatgtacttttgaatgcttcaatagtatttttcataatcatggaaggcactagagtgcagtgcatattgcacaaggttctttaatggcatcttgaatttgttagaacatggagttatgatatacaaccattcctatgtcttcatctaatagctaaagcttttggcgtagttggttaacatggtattggaatcttattttaaaggtccatcttattcgcaagtacaaggcaggtggacctgcgcattattcacgtgccaggctcaaagggattttgtatgagggggaatttttggaatataatatatagaatcattcatgtggttcacctatcatgtgtcaactcaagtttttgtcctagttgtattattgcagtgtctaaactaggcatgatcttctgtggaaggaaatttaacctaaggaacttgaatgcttcttgttctgagatctttattttttttctccaatctttttaatacttgaaaggttttagttgtatctataaagggtgcagaattagggggtcagcttttctgttgtaagcgtgaattaagtgcattttatgttgttccctattttatacatttgaactatcaaccatatcatttagcaaacttctttgttgctcagattcccatttattcttgtttcctctattttttagtggcaagttggagagttaaagagactgagaggatgatagcaatctgcacagaactcagaaaggtcttgctaattcctttatggtttctatattactggactttttacatctcactgacctactactgtcttggtatttcagctaggagcaacagttgaagaaggtcctgattactgtgtgattactccacctgagaaattgaatgtcacagctatagacacatatgatgaccacagaatggccatggcattctctcttgctgcttgtggggatgttccagtaaccatcaaggatcctggttgcaccaggaagacatttcctgactactttgaagtccttgagaggttaacaaagcactaa

SEQ ID NO: 13 - DNA, 4406 bp, Artificial Sequence
  pUbi3EPSPS chr 3 CDS
gtgaaaattttcacaagattttgaaagctctgaactcattagcacctgatttaatggttagaccaggctcaactgtgcctgcaaaaataagggaaagcacaaggttttatccttattttaaggtatgtgatcattatctaattataacaaaattataattataattgtgatggcatgtgcagcacttcataattttcttcgcaaagaatgtcgttctgatgaatttccagtggaacctactgacgagtcttcatcttcatcttcagtgttaccaaattacgaagacaatgatcatgaacccattgttcaaacacaagagcaggaacgagaagatgctaatatatggaggactaatataggttcagatatgtggagaaatgctaataattaggcgaacatgaagtgagaatcactttgttattatttttttaggcaataatgactttgttattaaaaggtttaaaatttctatcgtttttattttctttattcaaacattataatttatttatcatctttttcattcacttttgtaacttccgttatttttttgtttaaaatgtattaatctttcaaaatcttaaaaatccataaagtactttgaaatcttaaaaatctgtgttagaaatccattaaaatcttgggttaagaatcctgattgtaaaaagtcttttaaaaaaaatcttttaaaatcccacaaaatcaatacaatcccacataatcttttaaaatcttcaagattgtttttgtcaaaatattctctcaaaatcccaatccaatacaccccccttaattaacaaaaatgaatacgggctaacatggttatgggcttaggctgcctcattacccattacatgctctgttcctaaaaaaaagaagtttgggcctttattctccagtaagctttttaattgggctcaagaactttaggtatttaggtgagattcaatgaaaatttatacattaggcttgatttttttttttcaatttacgcaaaaatgaaagaaaaagtcacactctataaactttatttttttcaatattaatacattgttattgtggatatatcattagcggaaaagtaattctaataccagatttaattgactttgactaatttttttatgtgatcaaataatttttttagcgaaaaacttatatatatatatatatatatatatatatataagttttgttacttttccgcgtgatttttaatcaaactttggggataatttcccctccaattcagccaaaaaaaaaaaaaaactgacaccatatattattagtaggcaacttgttcgtaaatggtgtggcttatgcgaatggaaattggattcgttttctttaacatcattattgtttttgtcaatgagctatcttttagtcttatgttattggtgaatctgtccttaagttgcagcatttaacacatctcctcattagagaaaaaaattcttccctaaacgatagtaaaaacatctaataagaaataagaaagaaaaattaggaaaaagaaaagttcattaaaaaaatcttttggattatttttaaaaaaatatctaaatattttttaaatgaataattttatataaactgtaactaaaagtatacaagtaatgtatgttaacaaaatacttgaaaaatctactgaaaatatatcttacaaagtgaaattaaataagaaagaatttagtggaataattatgattttatttaaaaaataattattaaagatttttttgctccataataagaaaacttttcaattattcttttctggtccataataaaaaaaatctagcatgacagcttttccatagatttttaataatgtaaaagcagccgacttcaggcaatggatagtggggcccgtatcaacttcggacgctccacttgcaacggggtgggcccaatataacaacgacgtcgtaacagataaagcgaagcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacagatggcccaagtgagcagagtgcacaatcttgctcaaagcactcaaattttcggtcattcttccaatcccaacgaacccaaatcggcgaattcggtttcattgaggccacgcctttggggtccctcgaaatctcgcatcttggtgcacaaaactggaagccttatgggaaattttaatgcggggaagggaaattccggcatgtttaaggtttctgcctccgtcgccgccgccgcagagaagccttcgacggcgccggagatcgtgttggaacctatcaaagacatctcgggtaccatcacattgccagggtctaagtctctgtccaatcgaattttgcttcttgctgctctctctgagggaacaactgttgtagacaacttgctgtacagcgaggatattcattacatgcttggtgcattaaggacccttggactgcgtgtggaagacgaccaaacaaccaaacaagcaattgtggaaggctgtgggggattgtttcccactattaaagaatctaaagatgaaatcaatttattccttggaaatgctggtactgcgatgcgtcctttgacagcagctgtagttgctgcaggtggaaatgcaagctacgtacttgatggagtgccccgaatgagagagaggccaattggggatttggttgctggtcttaagcagctcggtgcagatgttgattgctttcttggcacaaactgtccacctgttcgtgtaaatgggaagggaggacttcctggcggaaaggtgaaactgtctggatcaattagcagtcaatacctaactgctttgcttatggcagctcctttagctcttggcgacgtggaaattgagattgttgataaactgatttctgttccatatgttgaaatgactctgaagttgatggagcgttttggagtttctgtggaacacagtggtaattgggataagttcttggtccatggaggtcaaaagtacaagtctcctggcaatgcttttgttgaaggtgatgcttcaagtgccagttacttcctagctggtgcagcagttactggtgggactatcactgttaatggctgtggcacaaacagtttacagggagatgtaaaatttgctgaagttcttgaaaagatgggagctaaggttacatggtcagagaacagtgtcaccgttactggaccaccacaagattcttctggtcaaaaagtcttgcaaggcattgatgtcaatatgaacaagatgccagatgttgccatgactcttgccgttgtcgcactatttgctaatggtcaaactgccatcagagatgtggcaagttggagagttaaagagactgagaggatgatagcaatctgcacagaactcagaaagctaggagcaacagttgaagaaggtcctgattactgtgtgattactccacctgagaaattgaatgtcacagctatagacacatatgatgaccacagaatggccatggcattctctcttgctgcttgtggggatgttccagtaaccatcaaggatcctggttgcaccaggaagacatttcccgactactttgaagtccttgagaggttcacaaggcactaa

SEQ ID NO: 14 - DNA, 10359 bp, Artificial Sequence
  pUbi3EPSPS chr 3 genomic sequence
gtgaaaattttcacaagattttgaaagctctgaactcattagcacctgatttaatggttagaccaggctcaactgtgcctgcaaaaataagggaaagcacaaggttttatccttattttaaggtatgtgatcattatctaattataacaaaattataattataattgtgatggcatgtgcagcacttcataattttcttcgcaaagaatgtcgttctgatgaatttccagtggaacctactgacgagtcttcatcttcatcttcagtgttaccaaattacgaagacaatgatcatgaacccattgttcaaacacaagagcaggaacgagaagatgctaatatatggaggactaatataggttcagatatgtggagaaatgctaataattaggcgaacatgaagtgagaatcactttgttattatttttttaggcaataatgactttgttattaaaaggtttaaaatttctatcgtttttattttctttattcaaacattataatttatttatcatctttttcattcacttttgtaacttccgttatttttttgtttaaaatgtattaatctttcaaaatcttaaaaatccataaagtactttgaaatcttaaaaatctgtgttagaaatccattaaaatcttgggttaagaatcctgattgtaaaaagtcttttaaaaaaaatcttttaaaatcccacaaaatcaatacaatcccacataatcttttaaaatcttcaagattgtttttgtcaaaatattctctcaaaatcccaatccaatacaccccccttaattaacaaaaatgaatacgggctaacatggttatgggcttaggctgcctcattacccattacatgctctgttcctaaaaaaaagaagtttgggcctttattctccagtaagctttttaattgggctcaagaactttaggtatttaggtgagattcaatgaaaatttatacattaggcttgatttttttttttcaatttacgcaaaaatgaaagaaaaagtcacactctataaactttatttttttcaatattaatacattgttattgtggatatatcattagcggaaaagtaattctaataccagatttaattgactttgactaatttttttatgtgatcaaataatttttttagcgaaaaacttatatatatatatatatatatatatatatataagttttgttacttttccgcgtgatttttaatcaaactttggggataatttcccctccaattcagccaaaaaaaaaaaaaaactgacaccatatattattagtaggcaacttgttcgtaaatggtgtggcttatgcgaatggaaattggattcgttttctttaacatcattattgtttttgtcaatgagctatcttttagtcttatgttattggtgaatctgtccttaagttgcagcatttaacacatctcctcattagagaaaaaaattcttccctaaacgatagtaaaaacatctaataagaaataagaaagaaaaattaggaaaaagaaaagttcattaaaaaaatcttttggattatttttaaaaaaatatctaaatattttttaaatgaataattttatataaactgtaactaaaagtatacaagtaatgtatgttaacaaaatacttgaaaaatctactgaaaatatatcttacaaagtgaaattaaataagaaagaatttagtggaataattatgattttatttaaaaaataattattaaagatttttttgctccataataagaaaacttttcaattattcttttctggtccataataaaaaaaatctagcatgacagcttttccatagatttttaataatgtaaaagcagccgacttcaggcaatggatagtggggcccgtatcaacttcggacgctccacttgcaacggggtgggcccaatataacaacgacgtcgtaacagataaagcgaagcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacagatggcccaagtgagcagagtgcacaatcttgctcaaagcactcaaattttcggtcattcttccaatcccaacgaacccaaatcggcgaattcggtttcattgaggccacgcctttggggtccctcgaaatctcgcatcttggtgcacaaaactggaagccttatgggaaattttaatgcggggaagggaaattccggcatgtttaaggtttctgcctccgtcgccgccgccgcagagaagccttcgacggcgccggagatcgtgttggaacctatcaaagacatctcgggtaccatcacattgccagggtctaagtctctgtccaatcgaattttgcttcttgctgctctctctgaggtaaagtttatttatttattttttctgtatccaaaaatgtaaattgttagtttgttaatttttgttagcagtgagagtcgaactacttggcttctttcttagtcatccaaccaaccttatatctccaaaatttatttattggttttttttggggttatgtggttaggaataggagtttgatgcgtggagtggattttgaatatttgattttttttttgtattattcagtgaaaatgaagcatcttgtcccatgaaagaaatggacacgaaattaagtggcgtatgatttagaatgatgatagaaatgtgtataggtggttttaatgtgtagcaataagcatattcaatatctggattgatttggacgtttctgtataaaggagtatgctagcaatgtgttaataatgtcttgttaaaagtatggatggctaaaataatcataaaaatcgagtgggagtagtatacatatctacaggaaatgtattaggtgaggcatttggcttctctattgcgagtaaggaacaagtaatctcagttaatgtgaaaatcaatggttgatattccaatacattcatgatgtgttatttacgggaacgcaatattgactgttgattttatctgcagctgagattatatactatatccttccaaagaaatctttgactcttgattatccaagtatggttgtattaccaattttagctctagaagataatccctcccccaaaacacaaattagaatggtgctgcaagttctgtgttactttcattctattatttttataacttttaattatttaatagatgtcttgtttggcataaactataatttattctgtttttttttatttacttatttagggaacaactgttgtagacaacttgctgtacagcgaggatattcattacatgcttggtgcattaaggacccttggactgcgtgtggaagacgaccaaacaaccaaacaagcaattgtggaaggctgtgggggattgtttcccactattaaagaatctaaagatgaaatcaatttattccttggaaatgctggtactgcgatgcgtcctttgacagcagctgtagttgctgcaggtggaaatgcaaggtctgttttttgttttgtttgttcatcatgatctctgaattgttcctcgtataactaatcacatcagactatgtgttcttcctccatcctgttataatctaaaaatctaatccagattagtcatccttctttaaatgaacctttaattatatctatgtatttatttaacatgtaaattagcttgtcaagtcaaagtctagcatatagatatactgattacactctgaggaatgcacctgagggtcttactcatgatctacttcaaccttgccactttcttcttttattattagatcacctatcatgattactggtttgagtctctaaatagaccatcttgatgttcaaaatatttcagctacgtacttgatggagtgccccgaatgagagagaggccaattggggatttggttgctggtcttaagcagctcggtgcagatgttgattgctttcttggcacaaactgtccacctgttcgtgtaaatgggaagggaggacttcctggcggaaaggtatggtttggatttccattagaataaggtggactaactttcctggagcaaaattctaatttaaaccctgtttccctctctttagaataagacactaagggtatgtttaggagttgggttttggcgagaaagggaagggagggcaattttttttctaataaatattctttaatttgataaaatttttaaacgaaggaatatgaagatagattagcataacttaatgttttaatcttttatttatttttataaatattatatacctctatttaaaaacaagatatttttcctccattccctttccctttgaaacctcagttccaaatataccgtacttgaattatattttggaaggtgtattggttggagacctttccttttcagaggttatccctcacctttattatagcctttctactctctcaatgagttcattgtgcattgagtcattgaacttcctgtgaaaagaaattgtttacttttctttatttgttacctacatccttattcctgttttaaaaaatactaagttttcttttagttatgccttcccctccttattctctccaaagaaaatataatagcgaatgttggtttatccattgtgaatcttattatcctatttcacatgcaggtgaaactgtctggatcaattagcagtcaatacctaactgctttgcttatggcagctcctttagctcttggcgacgtggaaattgagattgttgataaactgatttctgttccatatgttgaaatgactctgaagttgatggagcgttttggagtttctgtggaacacagtggtaattgggataagttcttggtccatggaggtcaaaagtacaagtaggtttctatgttttagcattacatcactttttagtatccaaaatgcaatgaaatcaaaactcatgttttctatcaggtctcctggcaatgcttttgttgaaggtgatgcttcaagtgccagttacttcctagctggtgcagcagttactggtgggactatcactgttaatggctgtggcacaaacagtttacaggtattcatgcttgacactattttctattatcttttattttatggtctatactgtatcattcctaattcaacccccttcttttctctttcccctcacttccaaaaaaatagagcttatcattgtattcttaatagctcttgtatgtcctttttcgtctcttgagcagattctaccgtgattatcctttttagtaaacttttttttgtgtgtcttttaaataattgatatcatcatgtgaactcacacatcatctctatcccttcctcattttattatatttcattaacagtgataaagataaaaataaattgtcaagtaaagcaagttggcatgtcagataatgatgcttcaattgcaaaattggtgaaagcttgcacagtacattagctttggtgacaattgtgatgtagatagccataacctcccatgcaacttattatgttgtttgcttaaacttcttttgcacttaaacacggattagagagagtgaggatgaacaataataacagacacaaagcttagttttataccacccggcacccagtggtgaggaccaagcagcatgagtgagatcaatggcagcatctgattgaagcttcttgtggtatttttggagatgaaatcttgaggcgaattagggcatctggcttaacaatagccattggttcaaatttgcttttaataaaattgattacagaatcaaattcttgaggaagccttcaagaactacatcatgatgttccctgtgcaaaacaggttcgccgacaaaagagaacatctacaagttcctttacacatgccaaaaattcctcaactgaacaattgccaagagtcatgttgcgaatttcaggttggagttgacgaaatttggtgcgtgtatgattctggaaatgagcatgaagttttttcctaagctcataaaagtgaatgcagccaagaaccctagataggattgaatcggagagaggcttgaagccaagataacagcaactgatcttgctgttcccatgctgcgtaggactcattgattctaccaagatcacaatcttctgtggtgagaaaatggggtgaattgtcaaccaatagagaaaatggtgaaggctgagtcttgatgataggcttgatttgcttgcgccgaagaagaacgttattgttgtagagtttctcagtgattgtatgagagtaagttacaggtgcgattggagatgacggacttgtggtgggaggagggtaagtgttaagtggtggaatgtgtatcagaggtggttgcccagccaaagaagctggttgcggtggtgcagtggaagccatgggttttgatgcttttgagagaaaacatatgaaactgaactgtaaacttttgtattattgattagctgatttggtagtacaagagaggaatatatatacagttttaatgtaactacctaaccaaacatgaggaactctccatacacagaaagacgtatacaaaggataacagacaaacagaaacttagatatactcatacagagaagaattacaggatgaatctgagaattttaattgggaaactgaggaaaatatcaagggtttcagaattcattttgacgataggcaaatcatggacttaacttagacctcaagggtacgcttgggagtgggggtttttagagggaaaagggaaggagggcaaacttttaattcctaataaatattctcttttttgatttttttttaaaataaaacaaataaacttatagattagcatacacttaatgtcctaatcttttatttatttgcatgagtttttttcttttatattctttctgtttctgttttctttctttagtgtctcttgtctttccttaattccttatcaataccttgtttatttttgaaaacttaattttcctcatttttttaacctataggctatactatagtatgtataactattcaaataaatagtagaaaatgtttcaaatattccccagaactgtttcagtgaacaaagttcaagccagaaaaaagaagtatatcttattgtgaagagtagtctcaagtggttaattgtcaaattgtatcattttagaatacaaaattagaagtggtttgattagtttatacgaacgattatttaaatagggttgatgatgtggttaattactgatcatatgtgaagactgtgagtcgatgaccagcccagtgcagtcattttaaattccattattttttaatttgtgcttatgttaaactttgtccttctttcaaagggagatgtaaaatttgctgaagttcttgaaaagatgggagctaaggttacatggtcagagaacagtgtcaccgttactggaccaccacaagattcttctggtcaaaaagtcttgcaaggcattgatgtcaatatgaacaagatgccagatgttgccatgactcttgccgttgtcgcactatttgctaatggtcaaactgccatcagagatggtatggtttagctgtttgtttgtgttcaagagcttgttttaacaaattgttttttttttaattatggttaatgaaattattataattacactaatattgaaattggatcactaaccatgtgaaatgcttgaatttttagcttgaaaacttcttttgtggacaatttttcttttatctatggattgtggggcatctatgttgttcctttaagcttcgtacaacctggtctgtgggctggtccagtccttcttactcatgacagtcttttgcagtaaactggttaaacttgggtgacctgttgacccacctagattggcctgttctttttaaataaaacttcaaaaggctacttataagatttcaacatgatacttgaatgatcacccttactctaaaggccattatctgttcagttatgttcttcttcttcttcttattatttttaatttatattattatccaacgatctgatctttgaacatgatgctggacaatcatcaaagctagtttggttacatcatataataccttttttttcatatattatttttcccataatatatttttgtgtttttttttcatacaatattattctgaaaaaaacgtgtttcgtttttttcctggcttgaatgctatttcaatgactgggccttattattttctggtttaggttatacaactgtcctccaattgacttttgaaggcttcaatagttttcttcttaatcatggaaggcactagagtgcatattgaatatgttagaacatggaaggcttaaatatctttttagtctttgcaatttaacgtttttttgcttttagtccttgcaaaattataatttttttttagtcctggctaattatgtttgttttgttttttgtccttatatgctttagataacactttttttgttactgttcaaagcactatctaaagtgctttaaggactaaacacaaaacaaacataatttgcaaggactgaaaaacaaaaaaataattttgcaaggacgaaaaaataaaaataaaaaacacaaaattgcaagaccaaaaatatatttaaaccaacatgaaattatggtataaaaccattcatacgtcttcatctaatagcttaagcttttgggatagttggttaacaatatggtattagagtcttatgatttttaggtggtctttgagttcaatccttgctgctcccagttctagttaatttctttttggtccacttcaaataaaaagttgaatttaagggtaaggcagctggacctgcacattaatcatgtgtcaggctcaaagggcttttgtatggggggcatgtttggaatataatatagaatcattcatgtggtttcacctatcaaggatcaactctagtttttgtcatacttgtattattacagtgtctaaactagacatgatctttttccggaaggaaatttaacttaaaaggaacttagatgcttcttgttctgtgatctttatttgtttattttttccaatctttttaacaattgaaaggttttagttgtatccaatctttttatacatttggactatctaccatatcatttagcaaacttgtttgctgctcagattctcctttggactatctaccatatcatttagcaaacctgttcactgctcagattctcatttattctttgtttcctctatttttcagtggcaagttggagagttaaagagactgagaggatgatagcaatctgcacagaactcagaaaggtgagtgtcttgctaattcctttatggtttcgatattactgaactttttacatctcactgacctactctgtcttggtatttcagctaggagcaacagttgaagaaggtcctgattactgtgtgattactccacctgagaaattgaatgtcacagctatagacacatatgatgaccacagaatggccatggcattctctcttgctgcttgtggggatgttccagtaaccatcaaggatcctggttgcaccaggaagacatttcccgactactttgaagtccttgagaggttcacaaggcactaa

SEQ ID NO: 15 - DNA, 10361 bp, Artificial Sequence
  pERF10EPSPS chr 1 genomic sequence
attgaatactacacataccaatcaattatttactcaagagaatttaatacccaagtcttctgcacacaaaagaccgcacctctgtgtgttgttatatattcgccataacacaacgcatcaacaaaacaagttgccctatgtcatgttgacaaagttgtcatgcaagcatcaatttgacatttcattaagactatcattaacacaatttgacaacacatcaacaaacaaatgaccatgccatgtttggaatattttatgagaagtcggatgttcacaccttcctagtgaaaaaacagaaaattaaaggaaggctatccttttcaattagatgatcgtttgtttacgatgggtgcaaagtgcaaacacatgcaccctaaattgtgaaataacactagcaataaaacgatttcaaactaaagcttcacctaaagtattaatagtttcgcaaataactttgttcttactcaccaaaaaatagtctttgttctagaatatagctagatcacattgtcaccgccatattcggttttatatcgaagtgttttgtcaagacaagatatatatttttcttcttttctagaatcctgcattgaagacgcaacatagtggaagagtgaagactcttgaagtttacgcatatttgcctggatctaggactaaggctatgtttcagagatcaaattagaataaaaataaataaataaattgctattgttattgtttaattaaaataaatttagataaattgtatttgatttcgttttatgtaatatttattttatcctatttaaatttactatttactttacaaatcatgaaataaaaattcataagtaatacccatccaaaattcagatttataaataccaactccccatacgagggtcatcaacaaagcctaacaaaggttataacatatttaacatagcaaatataaaaatactataataacctttgcatcatagaacccaccttaataatctctgtctgcacaaattttgatgcacgtggtaatttcagtaacagccctcccattgtgctttcccccattccaccaatcctcatcaccccacaaatcttatttcacagtgatatacacaaaaaatactataagaaaatataacattttattttgttccattcaacaagataaatgatttaagattatttctccaaactaaaattatgagccaccccgttcttaagggcgaaaataacataaaaaacgaaaggtacacaatcaacattatattgtagatttttttttttaatctttcctcgtcactaactctctgcgcttaaacttgggtgaaaatacatcatatcatgacttctcacaagttaaaatcatttaattttacaaaaaaaaaattatatataagttaattttaatttataaaaaaaaattattttattatacatacataataatatacacatatataacaagaaagtcattgaaactaagactttgcttgcacgttacagccgtagtttagctccaccaagtgaccaaatcctcatgtcatgtttcactttcttaaaacctcctcaactgtctgagaacaaagtcaactagacataatgatccataaagcttgaaatatggaaaattttacactcagtatattgcaaagttccttcataatcaaatcaagtaatacttcaccaagaaaaaaagtcaaataaaataaataaatactgcataaaagtgataattaaaacaaaaatccaaatctgagaaattacctttctacataatatggtataaaaaaaagttgaaaaatagcagatgaaaaagtgaaaggaaaagcacctcttttaagtatagaaagaaaaaaaaaggcaaggatggtgaaaaataggtaaaatagaaaagagaaacgagatttagttgaagtggagcaaggttactagggtgttccctagcgcagaaaatcgaaggcattgagaggataaagaataataattcctgaggcaagaggatgcttcgtggctccatcatagcaccagggttgaaagtcacatttctccccactatatatatccctttccactcaacttaaacacacaacacaaccctcttctctctttaccttcttaacgcaccaagcgaagcgaaaagcgctttctaacttaagtgatggcccaagtgagcagagtgcacaatcttgctcaaagcactcaaatttttggccattcttccaactccaacaaactcaaatcggtgaattcggtttcattgaggccacgcctttggggggcctcaaaatctcgcatcccgatgcataaaaatggaagctttatgggaaattttaatgtggggaagggaaattccggcgtgtttaaggtttctgcatcggtcgccgccgcagagaagccgtcaacgtcgccggagatcgtgttggaacccatcaaagacttctcgggtaccatcacattgccagggtccaagtctctgtccaatcgaattttgcttcttgctgctctctctgaggtgaagtttatttatttatttatttgtttgtttgttgttgggtgtgggaataggagtttgatgtgtagagtggattttgaatatttgatttttttttgtattattctgtgaaaatgaagcatcatgtcccatgaaagaaatggacacgaaattaagtggcttatgatgtgaaatgaggatagaaatgtgtgtagggttttttaatgggtagcaataagcatattcaatatctggattgatttggacgtttctgtataaaggagtatgctagcaatgtgttaatgtatggcttgctaaaatactcctaaaaatcaagtgggagtagtatacatatctacagcaaatgtattaggtgaggcatttggcttctctattgtaaggaacaaataatatcagttaatgtgaaaatcaatggttgatattccaatacattcatgatgtgttatttatatgtacctaatattgactgttgtttttctccgcaatgaccaagattatttattttatcctctaaagtgactaattgagttgcttactttagagaagttggacccattaggtgagagcgtggggggaactaatcttgaatatacaatctgagtcttgattatccaagtatggttgtatgaacaatgttagctctagaagataaaccctcccccaaaacacaaattagaatgacatttcaagttccatgtatgtcactttcattctattatttttacaacttttagttacttaacagatgtcttgttcagcataaattataatttattctgtttttttttagggaacaactgttgtagacaacttgttgtatagtgaggatattcattacatgcttggtgcattaaggacccttggactgcgtgtggaagatgacaaaacaaccaaacaagcaattgttgaaggctgtgggggattgtttcccactagtaaggaatctaaagatgaaatcaatttattccttggaaatgctggtactgcaatgcgtcctttgacagcagctgtggttgctgcaggtggaaatgcaaggtctgttttttttttttttgttcagcataatctttgaattgttcctcgtataactaatcacaacagagtacgtgttcttcttcctgttataatctaaaaatctcatccagattagtcatcctttcttcttaaaaggaacctttaattatcaatgtatttatttaatatttaaattagcttgtcaaagtctagcatatacatattttgattatattctgagaaatgcacctgagggtgttcctcatgatctacttcaacctctgttattattagattttctatcatgattactggtttgagtctctaagtagaccatcttgatgttcaaaatatttcagctacgtacttgatggggtgccccgaatgagagagaggccaattggggatttggttgctggtcttaagcaacttggtgcagatgttgattgctttcttggcacaaactgtccacctgttcgtgtaaatgggaagggaggacttcctggcggaaaggtatggtttggatttcatttagaataaggtggagtaactttcctggatcaaaattctaatttaagaagcctccctgttttcctctctttagaataagactaagggtaggtttaggagttgggttttggagagaaatggaagggagagcaatttttttcttcttctaataaatattctttaatttgatacattttttaagtaaaagaatataaagatagattagcataacttaatgttttaatcttttatttatttttataaatattatatacctgtctatttaaaaatcaaatatttgtcctccattccctttcccttcaaaacctcagttccaaatataccgtagttgaattatattttggaaggcctattggttggagacttttccttttcagagattatccctcacctttattatagcctttctatttttaaacttcatatagacgccattcttgttttaaaaaacactaagttttcttttagttataccttcccctccttattctctccaaagtaaatattgtagcaagtgttggttgatccattgtgaatcttattatcctatttaacatgcaggtgaaactgtctggatcagttagcagtcaatacttgactgctttgcttatggcagctcctttagctcttggtgatgtggaaattgagattgttgataaactgatttctgttccatatgttgaaatgactctgaagttgatggagcgttttggagtttctgtggaacacagtggtaattgggataggttcttggtccatggaggtcaaaagtacaagtaggtttctatgttttagtgttacatcacttttagtatccaaaatgcaatgaaattaaaactcatgtttcctattaggtctcctggcaatgcttttgttgaaggtgatgcttcaagtgccagttatttactagctggtgcagcaattactggtgggactatcactgttaatggctgtggcacaagcagtttacaggtattcatgaatgacactatcttctattatcttttattttatagcttacactatcattcctaattcaacccccttcttttctctttcccctcactcccaaaatagagcttatcattgtatccttaatagctcttttatgtgctgtttcatctcttgagcagattctacatgattatcctttttagtgaacttttttgtcttttaaataattgatatcatcatttgaacttgcacatcatctctatccttcctcattttattatatttcattaagagtgataaaattacattgtcaagtaaagcaagttagcgtatcagataatgatgcttcgattgcagaattggtgaaagcttgcacaattcattagcttagtgacaattgtgatgtagatagccataatcttccatgcaacttattatgctttttgtctaaaacttcttttgcacttaaacatgaattagagagttaggatgaacaatagtaacagacacaaagcttatttctagaccacccagtggtgaagacgaagcagcatgagtgagattaatggcagcatctgattgaagcttcttttggtattattggagatgaaatcttgagccaaattagggcttttgcctcctcaatagccattggttcaaaattgctttcaataaaatgattacagaatcaaattcttaagaaagaccttcaagaacaacatcaagatgtttcctgtgcaaaacaggctcacctacaaaagagtgtctacaagcacctttacacttgcaaaaaattccttaactgaacaattgccaagagtcatgttgcaaacttgagattggagttgattagatttggtgtgtgtgagattctggaaatgagcatgaagttttttgcaaagctcataaaatgaatgcagccaagaatcctagataggattgaatttgagagagaggcttgaagtcaagataaccgcaactgatcttgctgttcccatgctgcataggactcgttgattctaccaagataacaatcttctgtggtgagaatatgagtgggaattgtcaggcaacaaagaaaatgaaggctatgagcctttgttaaggtgcagagagaaaacagaaaatgaagaacttgtgtattgaaggaattgaaaagccaattacaattgtgttgcagagagatatttatagttctgaagtgtataactaactaaacagaatgctaccaacctagaggcagttgagaattgctcccttattacaatactctcacagccttgatgataggcttgatttgctgccaccgaagaagaacattattgttggagagtttttcagtgattgcatcagagaaagttacaagtgtgattggagatgaaggacttgcggtcgtcgaaggttaagtgttaagtggtggaatgtgtatcggaggtggttgcctagcccaagaagctggttgcagtggaagcggatgcggcagaagccatgggttcagatcagaactatgttctagataccatattagggttagctttcaagagaaaacttatgaaactgaactgtaaactgttgtattattgattagctgatctagtagtgatggaagcttgcttgcgaagcttctatggaggctggatcattgagcttcaatgaggtccttcaatggtgatttttcaccatggagatgcagcggaagataaatgaagaggtgagaggaggcgtcatccactaggaaataaactatggaagaaggagcttcaccaccaagagagtgtcttggataagaagcttagagaggaagcttcaatggaggaatagaaagagagagggggaacaaaaaattgaaggaggaaaagagggagagaagttgaactttgaagtgtgtctcacaagactcattcattaaagttacaacaagtgttacacatgcttctatttatagtctaggtaacttccttgagaagctagaacttaactacacacacctctctaataactaagataacctccttgagaaatttccttgagaagcttccttaagaagattcctagagaagctaaagcttagttacacacacctctctaataactaagttcacctccttgagatgagaagctagagccttagctacacacacccttataatagctaaactcactctattccaaaatacatgaaaatacaaaaaagttcctactacaaagattactcaaaatgtcctgaaatacaagactaaaactctatactactagaatggtcaaaatacaaggtccaaaagaaggaaaaacctattctaatatttacaaagagagtggacccaaccttagtccatgggctcagaaatctaccctgaggttcatgggaatcctagggtcttctttagtagctctagtccaattttcttggagtcttctatccaatatccttgggggtaggattgcatcatgtagtacaagagaggggaatatatatacagttgtaagttgtaactacctaatcaaacccaactgactcaccatacacagaaaaacaatgtacaaaggataatagaaaaactgaaactgagatatactcattacatagaagaattacatgatgaatctgaaaattttaattgggaaactgaggacaataacaaggctttcagagttcattttgacaataggtaaattatggacttcaacttagacctcaagggtatgcttgggagtggggttttagaggaaaaggggaaggagggaaattttttaattctagtaaatattctctattttgattaatttttaaaataaaacaaataaacttatagattagcattcacttaatgtcctgatcttttatttgtttcatgaagttttttatagcctttctgtttctgttaaaagaaattggagggtgttcataatctatatgcccatatatatgtattgtcctgttgtctgtgtttctgttttctttctttagtgtctcgcctttccttaattccttatcaacaccttgtttatttttgaaaacttaaatgtcctcattttttttacctgtactataactttccaaacaaatagtagaaaatgtttcaaatattccccagaactgtttcagtgaacaaagttcaagccagaaaaaaagcaagcagaggctaaagaagtatatcttattgtgaagagtaatctcaattggttaattatcgaattgtatcattttacaatagaaaattagaagtgggttgattagtttatacgaacaattatttaaatagggttgatggtgtggttaattactgatcatatgttaagactatgagtcgatgaccagcctcagtgcagtcattttaagttccattattttttaattgtgcttatgtttaactttgtccttcttttaaagggagatgtaaaatttgctgaagttcttgaaaagatgggagctaaggttacatggtcagagaacagtgtcactgtttctggaccaccacgagatttttctggtcgaaaagtcttgcgaggcattgatgtcaatatgaacaagatgccagatgttgccatgacacttgctgttgttgcactatttgctaatggtcccactgctataagagatggtatggtttagctgtttgtttttgctcaagagcttgttttcacatatttgtggtcaagagcggtttagctgtttttttttttaaattgtggttaatgaaattattataattatactaatattgaaattggatcactaaccatgtgaaatgcttgaatttttagcttgaaaacttcttttgtggatgatttttcttttatctatgtagtgtggggcatctatgttgttcctttaagctttgtgtcatagttgtacaacctggtctgtgggctggtccagttcttactcatgactgtcttttgcagcaaactggttaaacttgggtgacctgttgacccacccagattggcctgttcttttttaaataaaactccaaaaggctacttataagatttcaacatgatacttgaatgaccacccttactttaaaggtcattatgccatcagaccccttgattctctgttcagttatgttcttattcttattattttttaatttatattattatccaacgatctgatctttgtacatgatgctggacaatcatcaaagctagtttggttacatcatataataccttttttttcataaattattttttcccataatatatttatatgtatttttttaatacaatattctggggaaattttttttttctgttttcttttttctggtttgaatactatttcaatgactgggccttattattttctggtttgggttatacaactgtcctttgatgtacttttgaatgcttcaatagtatttttcataatcatggaaggcactagagtgcagtgcatattgcacaaggttctttaatggcatcttgaatttgttagaacatggagttatgatatacaaccattcctatgtcttcatctaatagctaaagcttttggcgtagttggttaacatggtattggaatcttattttaaaggtccatcttattcgcaagtacaaggcaggtggacctgcgcattattcacgtgccaggctcaaagggattttgtatgagggggaatttttggaatataatatatagaatcattcatgtggttcacctatcatgtgtcaactcaagtttttgtcctagttgtattattgcagtgtctaaactaggcatgatcttctgtggaaggaaatttaacctaaggaacttgaatgcttcttgttctgagatctttattttttttctccaatctttttaatacttgaaaggttttagttgtatctataaagggtgcagaattagggggtcagcttttctgttgtaagcgtgaattaagtgcattttatgttgttccctattttatacatttgaactatcaaccatatcatttagcaaacttctttgttgctcagattcccatttattcttgtttcctctattttttagtggcaagttggagagttaaagagactgagaggatgatagcaatctgcacagaactcagaaaggtcttgctaattcctttatggtttctatattactggactttttacatctcactgacctactactgtcttggtatttcagctaggagcaacagttgaagaaggtcctgattactgtgtgattactccacctgagaaattgaatgtcacagctatagacacatatgatgaccacagaatggccatggcattctctcttgctgcttgtggggatgttccagtaaccatcaaggatcctggttgcaccaggaagacatttcctgactactttgaagtccttgagaggttaacaaagcactaa

SEQ ID NO: 16 - DNA, 6301 bp, Glycine max
gtgaaaattttcacaagattttgaaagctctgaactcattagcacctgatttaatggttagaccaggctcaactgtgcctgcaaaaataagggaaagcacaaggttttatccttattttaaggtatgtgatcattatctaattataacaaaattataattataattgtgatggcatgtgcagcacttcataattttcttcgcaaagaatgtcgttctgatgaatttccagtggaacctactgacgagtcttcatcttcatcttcagtgttaccaaattacgaagacaatgatcatgaacccattgttcaaacacaagagcaggaacgagaagatgctaatatatggaggactaatataggttcagatatgtggagaaatgctaataattaggcgaacatgaagtgagaatcactttgttattatttttttaggcaataatgactttgttattaaaaggtttaaaatttctatcgtttttattttctttattcaaacattataatttatttatcatctttttcattcacttttgtaacttccgttatttttttgtttaaaatgtattaatctttcaaaatcttaaaaatccataaagtactttgaaatcttaaaaatctgtgttagaaatccattaaaatcttgggttaagaatcctgattgtaaaaagtcttttaaaaaaaatcttttaaaatcccacaaaatcaatacaatcccacataatcttttaaaatcttcaagattgtttttgtcaaaatattctctcaaaatcccaatccaatacaccccccttaattaacaaaaatgaatacgggctaacatggttatgggcttaggctgcctcattacccattacatgctctgttcctaaaaaaaagaagtttgggcctttattctccagtaagctttttaattgggctcaagaactttaggtatttaggtgagattcaatgaaaatttatacattaggcttgatttttttttttcaatttacgcaaaaatgaaagaaaaagtcacactctataaactttatttttttcaatattaatacattgttattgtggatatatcattagcggaaaagtaattctaataccagatttaattgactttgactaatttttttatgtgatcaaataatttttttagcgaaaaacttatatatatatatatatatatatatatatataagttttgttacttttccgcgtgatttttaatcaaactttggggataatttcccctccaattcagccaaaaaaaaaaaaaaactgacaccatatattattagtaggcaacttgttcgtaaatggtgtggcttatgcgaatggaaattggattcgttttctttaacatcattattgtttttgtcaatgagctatcttttagtcttatgttattggtgaatctgtccttaagttgcagcatttaacacatctcctcattagagaaaaaaattcttccctaaacgatagtaaaaacatctaataagaaataagaaagaaaaattaggaaaaagaaaagttcattaaaaaaatcttttggattatttttaaaaaaatatctaaatattttttaaatgaataattttatataaactgtaactaaaagtatacaagtaatgtatgttaacaaaatacttgaaaaatctactgaaaatatatcttacaaagtgaaattaaataagaaagaatttagtggaataattatgattttatttaaaaaataattattaaagatttttttgctccataataagaaaacttttcaattattcttttctggtccataataaaaaaaatctagcatgacagcttttccatagatttttaataatgtaaaagcagccgacttcaggcaatggatagtggggcccgtatcaacttcggacgctccacttgcaacggggtgggcccaatataacaacgacgtcgtaacagataaagcgaagcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacagatgcagatcttcgtcaagaccctcaccggcaagaccatcacccttgaggtggaaagctctgacaccatcgacaacgtcaaggccaagatccaggacaaggaaggaatccccccggaccagcaacgtctcattttcgccggaaagcaacttgaggacggccgtacccttgctgactacaacattcagaaggagagtactcttcacctcgtcctccgtctccgtggtggcatgcagatcttcgttaagacactcaccggcaagaccataaccctagaggttgaaagctccgacaccatcgataacgtcaaggccaagatccaggacaaggagggtatccccccggaccagcaacgtctcatcttcgccggaaagcagctcgaggacggccgcaccctcgccgactacaacatccagaaggaatcaacccttcacctcgtcctccgtctccgtggtggcatgcagatcttcgttaagaccctcaccggcaagactattaccctagaagtcgaaagctccgacaccatcgacaacgtcaaggctaagattcaggacaaggagggaatccccccagaccagcagaggctgatcttcgccgggaagcagctcgaggacggacgcacccttgctgactacaacatccagaaggagtcaactctccacttggtgttgcgtcttcgtggtggtatgcagattttcgtgaagactcttacgggtaagactattaccctcgaggtggagagctctgacaccattgacaatgtgaaggccaagattcaggacaaggaaggcatcccaccggaccagcagaggctgatttttgctggcaagcagctcgaggatggaaggaccctcgctgactacaacatccagaaggaatcaacccttcaccttgtcctccgtctccgtggggggttttaagctcgttgtgtaatgttggatgtgttcccaaaacatttgaagaactttgatgtttaatgggtctgtaataatgtcccttgaaaataagttcggtttgtgttgaactcaattgtgtcccattaataatagtactctaatatcccacctacgtttgttatgaatgtgtgaaatatgaaatgattaattgtcatatcgtgttgttttaatttgttctgaattggctagaggggacttaatatggattttttattcgatttgtgtggtcttccatgcttgtcatgaaggaaaaacagggatgagttgtgtgaaggatggtgatcatccttcgaattcgatggtattataggttgaagtcggtcattgatagatgttatgtaaaggcaacaatgaaatttgaacaaactgagatcattgtggtgatcaattgaaatgggaaggttcttcatcaactgtggagtattgatgggtaggagacaactagctcaaggcataagggttttttgtttcatacttaaaatcaatttcccatactatcaataagtttcaatataaaatttataattggaattaatttaaacacacatcttgtttacgtttaatgattatatctaactttaaaagaaataaacacttactgaatatctacaaaaattacctggaataaccaatgaataattttttattcatagcatatatattaagtttgtttggacccactcagcggggttcaatgtttaacacttatggtctgcatgaagtaccaattgcaccattaaagatgaaccacatcaatttgttgtcctccattttgtcttcgactgcagaccgtgacttggctgagcttagcttcaaattgaagtcttacttattacactgaatcacctcgtacgcgatgagccatgtttaaattttaataaatattataatagtaagctgggtttagattttctttttattgaatctatattagtataagcaatttaagtaaatgtgttttaaaaaaattttatcgcataagagtctagtgtatcataagtttcaccataaaatttgttgtaatagattataaagaatttattttaacaaacttcactaaggaattcagaaagtaagctaagaacagcttatacaaatcaggtcatttttatgcaatcaaataaagtttttaaaagttacactccttgaatcaattactctattcaaggacaatgaatttgttgataaaaaaaaattactcacccttgatattagcagaggaaacattttctgtaaatgtaaaattttgatacaacccactcttttcttttcttttatagagaaagtctctacactcgtctaagaatatagatacatttgcctttgaaaaattaaaatcttactaacatccacgcgatcacgcttcttattggtatgtctggatgaagaattttacaataaaagttttttttttcatttttgaattaaaggagttgtgcaatgttgtgagaaaaaaaaaactaggaaatattaaatgatttcttcataaagcatcgatttactacttacttttattttggtaaatcactccaataaataaataaatccctttacattcccataaataaataaataaaaagcatacataatttgtttagtatatatttaaaagtattatttatgagtgccacactcgcatgaaatacggcaaatattttctcaaaatttgtctaaacttttagaaatataataatatataacattgcttgtatattgatcatataaaaaaatttacaccatctttccattataattctacatatatattgaattttacgatgattaaccaatcattttaaaagttatactaacaacaaaacaaattatgatggcactggaacgacataatcttttaacgtgtataacagcattcagtcggtaacaatttttctttaagaaaaaaaaatccgcgaggctacgatctttttttaaaagcacttattacattatatacctggggtaagatttcacagacgcttttgtccaacgtgcatacccgctcgtgctcaagtcaatcttgggcttatctaactatccttgcggcgaaacttgatacataatattttattaaatccatcttttttaactctattcttaatacaagaaaacgaatgcttaatacttgacatgaattccaataaacaacctctttgaggggacaatcagtagcacaaaacaagcccccgatcatccaagttttaaggaaggcattaggtataaacctagtatgaataaatttttcaagtacttgtaggtaaataagtagaggtgaaataaatcagacatctataagatcaatccaacttatagacactttaaatttttagacggaaaaacttgtaaaaaagatttttttataaaaaattgaacaagttaattttaacttatgactgcagttttattcatttaatcaaccttattttcttcttccatttgttgaaaagcttgttcaaattggaccatagtgcatgttggaatgagaattttagcacgtaactgcacgttactcaaccaaacagcat

SEQ ID NO: 17 - DNA, 5111 bp, Glycine max
attgaatactacacataccaatcaattatttactcaagagaatttaatacccaagtcttctgcacacaaaagaccgcacctctgtgtgttgttatatattcgccataacacaacgcatcaacaaaacaagttgccctatgtcatgttgacaaagttgtcatgcaagcatcaatttgacatttcattaagactatcattaacacaatttgacaacacatcaacaaacaaatgaccatgccatgtttggaatattttatgagaagtcggatgttcacaccttcctagtgaaaaaacagaaaattaaaggaaggctatccttttcaattagatgatcgtttgtttacgatgggtgcaaagtgcaaacacatgcaccctaaattgtgaaataacactagcaataaaacgatttcaaactaaagcttcacctaaagtattaatagtttcgcaaataactttgttcttactcaccaaaaaatagtctttgttctagaatatagctagatcacattgtcaccgccatattcggttttatatcgaagtgttttgtcaagacaagatatatatttttcttcttttctagaatcctgcattgaagacgcaacatagtggaagagtgaagactcttgaagtttacgcatatttgcctggatctaggactaaggctatgtttcagagatcaaattagaataaaaataaataaataaattgctattgttattgtttaattaaaataaatttagataaattgtatttgatttcgttttatgtaatatttattttatcctatttaaatttactatttactttacaaatcatgaaataaaaattcataagtaatacccatccaaaattcagatttataaataccaactccccatacgagggtcatcaacaaagcctaacaaaggttataacatatttaacatagcaaatataaaaatactataataacctttgcatcatagaacccaccttaataatctctgtctgcacaaattttgatgcacgtggtaatttcagtaacagccctcccattgtgctttcccccattccaccaatcctcatcaccccacaaatcttatttcacagtgatatacacaaaaaatactataagaaaatataacattttattttgttccattcaacaagataaatgatttaagattatttctccaaactaaaattatgagccaccccgttcttaagggcgaaaataacataaaaaacgaaaggtacacaatcaacattatattgtagatttttttttttaatctttcctcgtcactaactctctgcgcttaaacttgggtgaaaatacatcatatcatgacttctcacaagttaaaatcatttaattttacaaaaaaaaaattatatataagttaattttaatttataaaaaaaaattattttattatacatacataataatatacacatatataacaagaaagtcattgaaactaagactttgcttgcacgttacagccgtagtttagctccaccaagtgaccaaatcctcatgtcatgtttcactttcttaaaacctcctcaactgtctgagaacaaagtcaactagacataatgatccataaagcttgaaatatggaaaattttacactcagtatattgcaaagttccttcataatcaaatcaagtaatacttcaccaagaaaaaaagtcaaataaaataaataaatactgcataaaagtgataattaaaacaaaaatccaaatctgagaaattacctttctacataatatggtataaaaaaaagttgaaaaatagcagatgaaaaagtgaaaggaaaagcacctcttttaagtatagaaagaaaaaaaaaggcaaggatggtgaaaaataggtaaaatagaaaagagaaacgagatttagttgaagtggagcaaggttactagggtgttccctagcgcagaaaatcgaaggcattgagaggataaagaataataattcctgaggcaagaggatgcttcgtggctccatcatagcaccagggttgaaagtcacatttctccccactatatatatccctttccactcaacttaaacacacaacacaaccctcttctctctttaccttcttaacgcaccaagcgaagcgaaaagcgctttctaacttaagtgatggcgaacgcagctgaagtttcagccttgaaccgcatcaaactgcatcttttgggtgaactctctccactggccactcccctaaactattttgatgaatcaaaccctagcccctctgaatcttccaattcccaatcttcttctgtttctcttaaccactacttcactgacctcttcgaattcgactccaaaccccaaataatcgacctccaaactcccaaaacactaacttcagctcagaagaaacctcaattgaatcggaaaccgtcgctgctaatcgctgttccaaagaagaccgagtggatccagttcgggagcccggatccgaacccggtgatggctgcgccggagaacctgccgcagaagaatcactacagaggtgtccggcagcggccctggggcaaattcgccgccgagatccgcgacccgaacaagcgcggctctagggtttggctcggaaccttcgacaccgccgtcgaagccgccaaggcctacgaccgagccgccttcagactccgcggctccaaggccatcctcaacttccccctcgaagttagcgccgtggcggagaccgtctccgtcgccgccgccgaaggcaacgtcgagagaaagcgccgccgcgaggaagaggaggtggtggtggaggaagtaaagccggtggtgaagaaggaaaagataacggaacaggatgtgagttgttttagggagatgccgttaacgccgtctatgtggaccgggttctgggacagtgacgtcaaggacattttcaacgttccgccgttgtcgccgttatctccttttggattttcaccgctcgtggcggtgtgaaatttctaatttagcactgcgttttttcgtgtttaagttacagcgtcctgtatggggatgagtacagtgtacatttgacgaatttgttaattcaacaatcttttattcacttttatttgctacatctacatggttcatttattggtcttttgggttaagtttaaatatttaaaaaagattaatcacttaaaacttgaaaactttaattgtataccgtgaatcatttgtgaatctcatcactgagggaaagatacaataataatgattacaagttttgcatacttttacctttgtttaaaaaatgaaacatccatgagcttaatctccttaagaatctattttcaagggcttaaaatcaattaagcatgttcagaactacttttaagaaatagacccgggttaatcgatttcaagatggtataaccgatcacaccatgtcgatcttatgactttttagttgaaaattacaagttactgttttaactgattacaagagaccgcagtcaattaaatcaagaacaagccacgctttatataagaataaacatcgctgtttcaatcgctttcatctatattactttgttattcttgagaaaatcatgagtttattattttaatcgattaccagcttaacctaatcaaataaaatgttattaaatttgtccagaacaactcagtcattttaattgattacttctccattttaataaattatttcagttagtttttgaaaaatataaaagccaattaggctctctttctctcataacccccctaagcaaaattttgaaattatcattctctttgagggttttgaacttcttcaagatagaagaaaaatatctttgcataactttttttaccccctttgttgttgatcattacggattagtttctacatgggttaagcaagctatatatagttgtgttcttcatggatcttgagataggatttctcaaaagggttgttcttgttacaagttttcacaagaagggtattaggattcttattgtagggttctagaaaaaagtgtgctgtaaaatttcattgtatttgagcaatgttcttgtattaaattctagaatctttaacggaattcaatcttgttagattgagagttggatgtagctctctatggttcaaagtgaaccaatataaattggtgtcttttttctctcattctcaaaccttgattcttgtatcatattattttaattgtcatcatatctttaacatagtaaataaatcattagttctataatattggtttgagttgactaccgtaaatcaacctgagtattcaattgatcaagaatataatggcatttttatatgtaccgtagtttagaaatacttagtgtaatcctatttctaattaataatagaaatataactgctttactccttaggtacattgtcaaacctcattaaatctttgttttcatgtgtgcatgattgatctatttctttatattttgattttgtgttgctaacacaaacatgccttctaaaatataaatttgagaaataatgtggtaaagtataatttcttaaagataggaatttattaattatgtgaaatactaaaaaagatgatttcaaccaatcctctgactagtaaacccctggaaaagagattaatatacaaaacatcaagaagaataagattttattaagccatcaactaaaattgacaagtgataataatacaacctttatgattggagatgttataaagaaaggtcatatgagtaagaataagttatttgtcaattttgctagcactaaaattaattagtttttgtcccttcttcctataatgtgagaaagtgttggatattgcattaacatgatgctaaccttataaaatatttaataatttttatattccttataagtagtatatgatttgtaacaaaaacttgataaaatcatttatatgggtgtcaattttaaattcatatttttaactttaaatgaaatatttaacatattatatttctagttttgacttggtctttgtagataaatcttatctcctcctagcttctactttgtctagatctatttgtcttcaacaaaaatcaattttatcttttacattttgtagcattctttctcttaacccttctttcattt

SEQ ID NO: 18 - DNA, 5378 bp, Artificial Sequence
  pUbi3Ubi3T2AmEPSPS chr 1 CDS
gtgaaaattttcacaagattttgaaagctctgaactcattagcacctgatttaatggttagaccaggctcaactgtgcctgcaaaaataagggaaagcacaaggttttatccttattttaaggtatgtgatcattatctaattataacaaaattataattataattgtgatggcatgtgcagcacttcataattttcttcgcaaagaatgtcgttctgatgaatttccagtggaacctactgacgagtcttcatcttcatcttcagtgttaccaaattacgaagacaatgatcatgaacccattgttcaaacacaagagcaggaacgagaagatgctaatatatggaggactaatataggttcagatatgtggagaaatgctaataattaggcgaacatgaagtgagaatcactttgttattatttttttaggcaataatgactttgttattaaaaggtttaaaatttctatcgtttttattttctttattcaaacattataatttatttatcatctttttcattcacttttgtaacttccgttatttttttgtttaaaatgtattaatctttcaaaatcttaaaaatccataaagtactttgaaatcttaaaaatctgtgttagaaatccattaaaatcttgggttaagaatcctgattgtaaaaagtcttttaaaaaaaatcttttaaaatcccacaaaatcaatacaatcccacataatcttttaaaatcttcaagattgtttttgtcaaaatattctctcaaaatcccaatccaatacaccccccttaattaacaaaaatgaatacgggctaacatggttatgggcttaggctgcctcattacccattacatgctctgttcctaaaaaaaagaagtttgggcctttattctccagtaagctttttaattgggctcaagaactttaggtatttaggtgagattcaatgaaaatttatacattaggcttgatttttttttttcaatttacgcaaaaatgaaagaaaaagtcacactctataaactttatttttttcaatattaatacattgttattgtggatatatcattagcggaaaagtaattctaataccagatttaattgactttgactaatttttttatgtgatcaaataatttttttagcgaaaaacttatatatatatatatatatatatatatatataagttttgttacttttccgcgtgatttttaatcaaactttggggataatttcccctccaattcagccaaaaaaaaaaaaaaactgacaccatatattattagtaggcaacttgttcgtaaatggtgtggcttatgcgaatggaaattggattcgttttctttaacatcattattgtttttgtcaatgagctatcttttagtcttatgttattggtgaatctgtccttaagttgcagcatttaacacatctcctcattagagaaaaaaattcttccctaaacgatagtaaaaacatctaataagaaataagaaagaaaaattaggaaaaagaaaagttcattaaaaaaatcttttggattatttttaaaaaaatatctaaatattttttaaatgaataattttatataaactgtaactaaaagtatacaagtaatgtatgttaacaaaatacttgaaaaatctactgaaaatatatcttacaaagtgaaattaaataagaaagaatttagtggaataattatgattttatttaaaaaataattattaaagatttttttgctccataataagaaaacttttcaattattcttttctggtccataataaaaaaaatctagcatgacagcttttccatagatttttaataatgtaaaagcagccgacttcaggcaatggatagtggggcccgtatcaacttcggacgctccacttgcaacggggtgggcccaatataacaacgacgtcgtaacagataaagcgaagcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacagatgcagatcttcgtcaagaccctcaccggcaagaccatcacccttgaggtggaaagctctgacaccatcgacaacgtcaaggccaagatccaggacaaggaaggaatccccccggaccagcaacgtctcattttcgccggaaagcaacttgaggacggccgtacccttgctgactacaacattcagaaggagagtactcttcacctcgtcctccgtctccgtggtggcatgcagatcttcgttaagacactcaccggcaagaccataaccctagaggttgaaagctccgacaccatcgataacgtcaaggccaagatccaggacaaggagggtatccccccggaccagcaacgtctcatcttcgccggaaagcagctcgaggacggccgcaccctcgccgactacaacatccagaaggaatcaacccttcacctcgtcctccgtctccgtggtggcatgcagatcttcgttaagaccctcaccggcaagactattaccctagaagtcgaaagctccgacaccatcgacaacgtcaaggctaagattcaggacaaggagggaatccccccagaccagcagaggctgatcttcgccgggaagcagctcgaggacggacgcacccttgctgactacaacatccagaaggagtcaactctccacttggtgttgcgtcttcgtggtggtatgcagattttcgtgaagactcttacgggtaagactattaccctcgaggtggagagctctgacaccattgacaatgtgaaggccaagattcaggacaaggaaggcatcccaccggaccagcagaggctgatttttgctggcaagcagctcgaggatggaaggaccctcgctgactacaacatccagaaggaatcaacccttcaccttgtcctccgtctccgtggggggtttgagggcagaggaagtcttctaacatgcggtgacgtggaggagaatcccggcccttctagaatggcccaagtgagcagagtgcacaatcttgctcaaagcactcaaatttttggccattcttccaactccaacaaactcaaatcggtgaattcggtttcattgaggccacgcctttggggggcctcaaaatctcgcatcccgatgcataaaaatggaagctttatgggaaattttaatgtggggaagggaaattccggcgtgtttaaggtttctgcatcggtcgccgccgcagagaagccgtcaacgtcgccggagatcgtgttggaacccatcaaagacttctcgggtaccatcacattgccagggtccaagtctctgtccaatcgaattttgcttcttgctgctctctctgagggaacaactgttgtagacaacttgttgtatagtgaggatattcattacatgcttggtgcattaaggacccttggactgcgtgtggaagatgacaaaacaaccaaacaagcaattgttgaaggctgtgggggattgtttcccactagtaaggaatctaaagatgaaatcaatttattccttggaaatgctggtattgcaatgcgttctttgacagcagctgtggttgctgcaggtggaaatgcaagctacgtacttgatggggtgccccgaatgagagagaggccaattggggatttggttgctggtcttaagcaacttggtgcagatgttgattgctttcttggcacaaactgtccacctgttcgtgtaaatgggaagggaggacttcctggcggaaaggtgaaactgtctggatcagttagcagtcaatacttgactgctttgcttatggcagctcctttagctcttggtgatgtggaaattgagattgttgataaactgatttctgttccatatgttgaaatgactctgaagttgatggagcgttttggagtttctgtggaacacagtggtaattgggataggttcttggtccatggaggtcaaaagtacaagtctcctggcaatgcttttgttgaaggtgatgcttcaagtgccagttatttactagctggtgcagcaattactggtgggactatcactgttaatggctgtggcacaagcagtttacagggagatgtaaaatttgctgaagttcttgaaaagatgggagctaaggttacatggtcagagaacagtgtcactgtttctggaccaccacgagatttttctggtcgaaaagtcttgcgaggcattgatgtcaatatgaacaagatgccagatgttgccatgacacttgctgttgttgcactatttgctaatggtcccactgctataagagatgtggcaagttggagagttaaagagactgagaggatgatagcaatctgcacagaactcagaaagctaggagcaacagttgaagaaggtcctgattactgtgtgattactccacctgagaaattgaatgtcacagctatagacacatatgatgaccacagaatggccatggcattctctcttgctgcttgtggggatgttccagtaaccatcaaggatcctggttgcaccaggaagacatttcctgactactttgaagtccttgagaggttaacaaagcactaa

SEQ ID NO: 19 - DNA, 16 bp, Glycine max
tgtcctccgtctccgt

SEQ ID NO: 20 - DNA, 16 bp, Glycine max
ccaacattacacaact

SEQ ID NO: 21 - DNA, 16 bp, Glycine max
cttccatgcttgtcat

SEQ ID NO: 22 - DNA, 16 bp, Glycine max
ccttcacacaactcat

SEQ ID NO: 23 - DNA, 16 bp, Glycine max
tcatcaactgtggagt

SEQ ID NO: 24 - DNA, 16 bp, Glycine max
gccttgagctagttgt

SEQ ID NO: 25 - DNA, 49 bp, Glycine max
ttgtcctccgtctccgtggggggttttaagctcgttgtgtaatgttgga

SEQ ID NO: 26 - DNA, 49 bp, Glycine max
tcttccatgcttgtcatgaaggaaaaacagggatgagttgtgtgaagga

SEQ ID NO: 27 - DNA, 49 bp, Glycine max
ttcatcaactgtggagtattgatgggtaggagacaactagctcaaggca

SEQ ID NO: 28 - DNA, 3444 bp, Glycine max
ctaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacagatgcagatcttcgtcaagaccctcaccggcaagaccatcacccttgaggtggaaagctctgacaccatcgacaacgtcaaggccaagatccaggacaaggaaggaatccccccggaccagcaacgtctcattttcgccggaaagcaacttgaggacggccgtacccttgctgactacaacattcagaaggagagtactcttcacctcgtcctccgtctccgtggtggcatgcagatcttcgttaagacactcaccggcaagaccataaccctagaggttgaaagctccgacaccatcgataacgtcaaggccaagatccaggacaaggagggtatccccccggaccagcaacgtctcatcttcgccggaaagcagctcgaggacggccgcaccctcgccgactacaacatccagaaggaatcaacccttcacctcgtcctccgtctccgtggtggcatgcagatcttcgttaagaccctcaccggcaagactattaccctagaagtcgaaagctccgacaccatcgacaacgtcaaggctaagattcaggacaaggagggaatccccccagaccagcagaggctgatcttcgccgggaagcagctcgaggacggacgcacccttgctgactacaacatccagaaggagtcaactctccacttggtgttgcgtcttcgtggtggtatgcagattttcgtgaagactcttacgggtaagactattaccctcgaggtggagagctctgacaccattgacaatgtgaaggccaagattcaggacaaggaaggcatcccaccggaccagcagaggctgatttttgctggcaagcagctcgaggatggaaggaccctcgctgactacaacatccagaaggaatcaacccttcaccttgtcctccgtctccgtggggggttttaagctcgttgtgtaatgttggatgtgttcccaaaacatttgaagaactttgatgtttaatgggtctgtaataatgtcccttgaaaataagttcggtttgtgttgaactcaattgtgtcccattaataatagtactctaatatcccacctacgtttgttatgaatgtgtgaaatatgaaatgattaattgtcatatcgtgttgttttaatttgttctgaattggctagaggggacttaatatggattttttattcgatttgtgtggtcttccatgcttgtcatgaaggaaaaacagggatgagttgtgtgaaggatggtgatcatccttcgaattcgatggtattataggttgaagtcggtcattgatagatgttatgtaaaggcaacaatgaaatttgaacaaactgagatcattgtggtgatcaattgaaatgggaaggttcttcatcaactgtggagtattgatgggtaggagacaactagctcaaggcataagggttttttgtttcatacttaaaatcaatttcccatactatcaataagtttcaatataaaatttataattggaattaatttaaacacacatcttgtttacgtttaatgattatatctaactttaaaagaaataaacacttactgaatatctacaaaaattacctggaataaccaatgaataattttttattcatagcatatatattaagtttgtttggacccactcagcggggttcaatgtttaacacttatggtctgcatgaagtaccaattgcaccattaaagatgaaccacatcaatttgttgtcctccattttgtcttcgactgcagaccgtgacttggctgagcttagcttcaaattgaagtcttacttattacactgaatcacctcgtacgcgatgagccatgtttaaattttaataaatattataatagtaagctgggtttagattttctttttattgaatctatattagtataagcaatttaagtaaatgtgttttaaaaaaattttatcgcataagagtctagtgtatcataagtttcaccataaaatttgttgtaatagattataaagaatttattttaacaaacttcactaaggaattcagaaagtaagctaagaacagcttatacaaatcaggtcatttttatgcaatcaaataaagtttttaaaagttacactccttgaatcaattactctattcaaggacaatgaatttgttgataaaaaaaaattactcacccttgatattagcagaggaaacattttctgtaaatgtaaaattttgatacaacccactcttttcttttcttttatagagaaagtctctacactcgtctaagaatatagatacatttgcctttgaaaaattaaaatcttactaacatccacgcgatcacgcttcttattggtatgtctggatgaagaattttacaataaaagttttttttttcatttttgaattaaaggagttgtgcaatgttgtgagaaaaaaaaaactaggaaatattaaatgatttcttcataaagcatcgatttactacttacttttattttggtaaatcactccaataaataaataaatccctttacattcccataaataaataaataaaaagcatacataatttgtttagtatatatttaaaagtattatttatgagtgccacactcgcatgaaatacggcaaatattt

SEQ ID NO: 29 - protein, 19 aa, Glycine max
GNAGTAMRPLTAAVVAAGG

SEQ ID NO: 30 - protein, 525 aa, Artificial Sequence
  Modified Glyma01g33660 GmEPSPS Chr 1 protein
MAQVSRVHNLAQSTQIFGHSSNSNKLKSVNSVSLRPRLWGASKSRIPMHKNGSFMGNFNVGKGNSGVFKVSASVAAAEKPSTSPEIVLEPIKDFSGTITLPGSKSLSNRILLLAALSEGTTVVDNLLYSEDIHYMLGALRTLGLRVEDDKTTKQAIVEGCGGLFPTSKESKDEINLFLGNAGIAMRSLTAAVVAAGGNASYVLDGVPRMRERPIGDLVAGLKQLGADVDCFLGTNCPPVRVNGKGGLPGGKVKLSGSVSSQYLTALLMAAPLALGDVEIEIVDKLISVPYVEMTLKLMERFGVSVEHSGNWDRFLVHGGQKYKSPGNAFVEGDASSASYLLAGAAITGGTITVNGCGTSSLQGDVKFAEVLEKMGAKVTWSENSVTVSGPPRDFSGRKVLRGIDVNMNKMPDVAMTLAVVALFANGPTAIRDVASWRVKETERMIAICTELRKLGATVEEGPDYCVITPPEKLNVTAIDTYDDHRMAMAFSLAACGDVPVTIKDPGCTRKTFPDYFEVLERLTKH

SEQ ID NO: 31 - protein, 526 aa, Artificial Sequence
  Modified Glyma03g03190 GmEPSPS Chr 3 protein
MAQVSRVHNLAQSTQIFGHSSNPNEPKSANSVSLRPRLWGPSKSRILVHKTGSLMGNFNAGKGNSGMFKVSASVAAAAEKPSTAPEIVLEPIKDISGTITLPGSKSLSNRILLLAALSEGTTVVDNLLYSEDIHYMLGALRTLGLRVEDDQTTKQAIVEGCGGLFPTIKESKDEINLFLGNAGIAMRSLTAAVVAAGGNASYVLDGVPRMRERPIGDLVAGLKQLGADVDCFLGTNCPPVRVNGKGGLPGGKVKLSGSISSQYLTALLMAAPLALGDVEIEIVDKLISVPYVEMTLKLMERFGVSVEHSGNWDKFLVHGGQKYKSPGNAFVEGDASSASYFLAGAAVTGGTITVNGCGTNSLQGDVKFAEVLEKMGAKVTWSENSVTVTGPPQDSSGQKVLQGIDVNMNKMPDVAMTLAVVALFANGQTAIRDVASWRVKETERMIAICTELRKLGATVEEGPDYCVITPPEKLNVTAIDTYDDHRMAMAFSLAACGDVPVTIKDPGCTRKTFPDYFEVLERFTRH